(19) United States
(12) Patent Application Publication
(10) Pub. No.: US 2023/0158072 A1
(43) Pub. Date: May 25, 2023

(54) COMBINATION THERAPY INVOLVING ANTI-CD39 ANTIBODIES AND ADOPTIVE CELL THERAPY

(71) Applicant: Trishula Therapeutics, INc., South San Francisco, CA (US)

(72) Inventors: Courtney Beers, South San Francisco, CA (US); Achim Moesta, South San Francisco, CA (US); Nicole Malandro, South San Francisco, CA (US)

(21) Appl. No.: 17/916,503

(22) PCT Filed: Apr. 2, 2021
(86) PCT No.: PCT/US2021/025661

§ 371 Date: Sep. 30, 2022

Related U.S. Application Data
(60) Provisional application No. 63/005,099, filed on Apr. 3, 2020.

(51) Int. Cl. A61K 35/17; C07K 16/28; A61P 35/00

(52) U.S. Cl. CPC A61K 35/17; C07K 16/2896; A61P 35/00

(57) ABSTRACT

Provided is an antibody combination therapy with an anti-CD39 antibody and an adoptive cell therapy.

### FIELD

Provided herein are combination therapies involving antibodies with binding specificity for CD39 and adoptive cell therapy.

### BACKGROUND

Human CD39 is a 510-amino acid protein with seven potential N-linked glycosylation sites and 11 cysteine residues. CD39 is an integral membrane protein that phosphohydrolyzes ATP to yield ADP and AMP. Structurally, it is characterized by two transmembrane domains, small cytoplasmic domains, and a large extracellular hydrophobic domain. CD39 becomes catalytically active upon localization to the cell surface.

CD39 is constitutively expressed in spleen, thymus, lung, and placenta and in these tissues it is associated primarily with endothelial cells and immune cell populations, such as B cells, natural killer (NK) cells, dendritic cells (DC), Langerhans cells, monocytes, macrophages, mesangial cells, neutrophils, and regulatory T cells (Tregs). Expression of CD39 on CD8⁺ and CD4⁺ T cells can also be induced within the tumor microenvironment. Given that CD39, along with other enzymes, degrades ATP, ADP, and AMP to adenosine, CD39 is a checkpoint, an immunological switch that shifts ATP-driven pro-inflammatory immune cell activity toward an anti-inflammatory state mediated by adenosine.

The expression of CD39 is increased in many solid tumors. For example CD39 expression is increased in colorectal cancer, head and neck cancer, pancreatic cancer, bladder cancer, brain cancer, breast cancer, gastric cancer, hepatocellular carcinoma, lung cancer, non-small cell lung cancer, chronic lymphocytic leukemia, lymphoma, melanoma, ovarian cancer, and prostate cancer. Increased CD39 expression suggests that the enzyme is involved in the development and progression of malignancies. Expression of CD39 in solid tumors may be found on the tumor epithelium, on infiltrating leukocyte populations, or on the vascular endothelium.

Adoptive cell therapy is an immunotherapy using a subject's own immune cells (or a donor's immune cells) to treat diseases such as, for example, cancer or a viral infection. In adoptive cell therapy, T cells are isolated based upon their ability to expand in response to tumor or are genetically modified to target certain molecules on cells, such as antigens on cancer cells. The tumor reactive T cells are then expanded and infused back into the subject.

One form of adoptive cell therapy called TIL therapy involves tumor infiltrating lymphocytes. In TIL therapy, tumor infiltrating lymphocytes that have penetrated a tumor are collected from a tumor biopsy taken from a subject and expanded in vitro for re-infusion into the patient. The tumor infiltrating lymphocytes are actively engaged in tumor destruction. In one method, following excision of the biopsy, DNA isolated from the tumor is sequenced to identify mutations found in the cancer that are recognized as neoantigens.

In this method, mutated neoantigens are inserted into autologous dendritic cells, which are co-cultured with the tumor infiltrating lymphocytes. Tumor infiltrating lymphocytes are then assayed for neoantigen recognition. Those tumor infiltrating lymphocytes that recognize the neoantigen are then selected, expanded, and transfused back into the subject. In other methods, the T cells are expanded in number due to their capacity to recognize the tumor biopsy from which they were isolated and are infused back into the patient.

Another approach called TCR therapy involves engineering a subject's or donor's T cells to express a specific T-cell receptor (“TCR”). The T cell receptor is a heterodimer consisting of two subunits, TCRα and TCRβ. Each subunit contains a constant region that anchors the receptor to the cell membrane and a hypervariable region that functions in antigen recognition. TCRs can recognize tumor specific proteins on the inside and outside of cells.

In TCR therapy, T cells are harvested from a subject's or donor's blood. The T cells are genetically modified in the laboratory to express a new T cell receptor. The T cells are expanded in number and infused back into the subject. The T cells with the new T cell receptor may target a patient's cancer.

In chimeric antigen receptor (“CAR”) T cell therapy (“CAR-T therapy”), one or more parts of a T cell receptor is changed into an antigen binding moiety, such as an antibody or antibody fragment. A cancer associated antigen (tumor association antigen or “TAA”) is often expressed by tumors. The antibody or antibody fragment is targeted to the TAA. T cells targeted to a TAA may directly attack cancer cells.

In CAR-T therapy, T cells are harvested from a subject's or donor's blood and genetically modified to express a chimeric antigen receptor. T cells are expanded in number and infused back into the subject. CAR-T modifications target T cells specifically to the subject's cancer.

Checkpoint inhibitors have shown considerable promise in the treatment of diseases, such as cancer. Adoptive cell therapy has likewise shown considerable promise. An inhibitor of a checkpoint, such as a CD39 antibody, and adoptive cell therapy administered in combination may show even greater promise than either of the therapies alone.

### SUMMARY

Provided herein are methods and pharmaceutical compositions for treatment of a subject suffering from cancer, the composition comprising an antibody which binds to CD39 and an adoptive cell therapy.

A first aspect provides a therapeutic composition comprising an antibody which binds to CD39 and an adoptive cell therapy composition. In some embodiments, the adoptive cell therapy comprises or consists of TIL therapy, TCR therapy, and/or CAR therapy.

In some embodiments, the adoptive cell therapy composition comprises or consists of immune effector cells. In some embodiments, the immune effector cells comprise or consist of NK cells, T cells, dendritic cells, macrophages, peripheral blood mononuclear cells (PBMCs), αβ T cells, γδ T cells, regulatory T cells, NK T cells or a combination thereof. In some embodiments, the adoptive cell therapy composition comprises or consists of immune effector cells comprising or consisting of engineered cells expressing a heterologous TCR and/or a CAR. In some embodiments, the engineered cells are T cells.

In some embodiments, the engineered cells express a CAR comprising an extracellular binding moiety that specifically binds a tumor-associated antigen (TAA). In some embodiments, the TAA is a B cell antigen. In some embodiments, the CAR comprises an anti-CD19 binding moiety. In some embodiments, the TAA is a melanoma antigen. In some embodiments, the melanoma antigen is gp100.

In some embodiments, the CAR comprises an extracellular binding moiety, a transmembrane domain, and an intracellular domain that triggers the activation and/or proliferation of lymphocytes. In some embodiments, the intracellular domain comprises a CD3ζ signaling domain. In some embodiments, the CAR further comprises at least one costimulatory domain or a combination of costimulatory domains with other costimulatory domains and/or inhibitory domains. In some embodiments, the CAR comprises a CD28 or 4-1BB costimulatory domain.

In some embodiments, the engineered cells express the heterologous TCR. In some embodiments, the heterologous TCR specifically binds a TAA. In some embodiments, the TAA is a melanoma antigen. In some embodiments, TAA is a peptide derived from gp100. In some embodiments, the heterologous TCR is a TCR recognizing gp100.

In some embodiments, the antibody which binds to CD39 comprises a heavy chain variable region (VH) and a light chain variable region (VL), the VH and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

A second aspect provides a method of treating a subject in need thereof, the method comprising:

(a) administering to the subject an adoptive cell therapy composition; and

(b) simultaneously or sequentially administering to the subject an anti-CD39 antibody or immunologically active fragment thereof, wherein the method comprises performing (a) before (b), performing (b) before (a), performing (a) and (b) simultaneously, or a combination thereof. In some embodiments, the method further comprises administering the adoptive cell therapy composition and waiting for cells of the composition to migrate to a target cell or tissue of the subject before performing (b). In some embodiments, step (b) further comprises administering the anti-CD39 antibody or immunologically active fragment thereof at a dose schedule.

In some embodiments, the dose schedule comprises administering the anti-CD39 antibody or immunologically active fragment thereof weekly, twice-monthly, monthly, or every two months. In some embodiments, the method further comprises at least one additional step of administering the adoptive cell therapy composition.

In some embodiments, the adoptive cell therapy comprises or consists of TIL therapy, TCR therapy, and/or CAR therapy.

In some embodiments, the adoptive cell therapy composition comprises NK cells, T cells, dendritic cells, macrophages, PBMCs, αβT cells, γδ T cells, regulatory T cells, NK T cells or a combination thereof. In some embodiments, the adoptive cell therapy composition comprises engineered cells expressing a heterologous TCR and/or a CAR.

In some embodiments, the engineered cells express a CAR comprising an extracellular binding moiety that specifically binds a TAA. In some embodiments, the TAA is a B cell antigen. In some embodiments, the CAR comprises an anti-CD19 binding moiety. In some embodiments, the TAA is a melanoma antigen. In some embodiments, the melanoma antigen is gp100.

In some embodiments, the CAR comprises an extracellular binding moiety, a transmembrane domain, and an intracellular domain that triggers the activation and/or proliferation of lymphocytes. In some embodiments, the intracellular domain comprises a CD3ζ signaling domain. In some embodiments, the CAR further comprises at least one costimulatory domain or a combination of costimulatory domains with other costimulatory domains and/or inhibitory domains. In some embodiments, the CAR comprises a CD28 or 4-1BB costimulatory domain.

In some embodiments, the engineered cells express the heterologous TCR. In some embodiments, the heterologous TCR specifically binds a TAA. In some embodiments, the TAA is a melanoma antigen. In some embodiments, the TAA is gp100. In some embodiments, the heterologous TCR is a pmel-1 TCR.

In some embodiments, the antibody which binds to CD39 comprises a heavy chain variable region (VH) and a light chain variable region (VL), the VH and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 SEQ ID NOs: 289-293.

A third aspect provides a method of producing an adoptive cell therapy composition, the method comprising:

(a) providing a first population of cells; and

(b) culturing the first population of cells under conditions sufficient to generate a therapeutically effective amount of cells for adoptive cell therapy in the presence of an anti-CD39 antibody or immunologically active fragment thereof. In some embodiments, the first population of cells comprises cells obtained from a subject in need of adoptive cell therapy treatment. In some embodiments, the first population of cells comprises cells obtained from a donor subject or cell line and the adoptive cell therapy composition comprises cells produced for treatment of a recipient subject. In some embodiments, the adoptive cell therapy comprises or consists of TIL therapy, TCR therapy, and/or CAR therapy.

In some embodiments, the method further comprises engineering the first population of cells, or a portion thereof, and/or engineering a second population of cells, or a portion thereof, wherein the second population of cells are produced by expanding the first population of cells before or during step (b), wherein engineering the first population and/or engineering the second population comprises genetically modifying the cells to express a CAR or a heterologous TCR.

In some embodiments, the anti-CD39 antibody comprises a heavy chain variable region (VH) and a light chain variable region (VL), the VH and/or VL together comprising 1, 2, 3, 4, 5, or 6 of: (a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

In some embodiments, the cancer is a solid cancer. In some embodiments, the cancer is a hematological cancer. In some embodiments, the cancer is selected from the group consisting of metastatic non-small cell lung cancer (NSCLC), metastatic head and neck squamous cell carcinoma (HNSCC), acute lymphoblastic leukemia (ALL), multiple myeloma (MM), acute myeloid leukemia (AML), chronic lymphocytic leukemia (CLL), diffuse large B-cell lymphoma (DLBCL), melanoma, renal cell carcinoma, metastatic cutaneous squamous cell carcinoma, Hodgkin's lymphoma, non-Hodgkin's lymphoma (NHL), and unresectable or metastatic solid tumor with DNA mismatch repair deficiencies or a microsatellite instability-high state. In some embodiments, the subject is a human subject.

### BRIEF DESCRIPTION OF THE DRAWINGS

FIG. 1 depicts a representation of an antibody which binds CD39 combined with an adoptive cell therapy, such as CAR-T therapy.

FIG. 2 depicts that administration of anti-CD39 antibodies during treatment with CAR-T cells in a mouse xenograft model increases the percentage of CD4+, CD8+, and CD3+ T cells in the blood as measured by flow cytometry at multiple study timepoints capturing CAR-T cell expansion phases and persistence phases. Percentages are shown from individuals, with mean percentage indicated ±SEM. Statistics were determined by unpaired t-test (p*<0.05, p**<0.005, p***<0.001).

FIG. 3 depicts that administration of anti-CD39 antibodies during treatment with CAR-T cells in a mouse xenograft model increases the engraftment of CD4+, CD8+, and CD3+ T cells in peripheral organs at study termination on day 61 as measured by flow cytometry. Percentages are shown from individuals, with mean percentage indicated ±SEM. Statistics were determined by unpaired t-test (p*<0.05, p**<0.005, p***<0.001).

FIG. 4 depicts decreased tumor burden in a mouse xenograft model using luciferase expressing Raji tumor cells in groups treated with CAR-T therapy in combination with anti-CD39 antibodies as quantified by bioluminescent flux (p/s). (A) shows plotted individual animal flux measurements and (B) shows geometric group mean±SEM flux measurements. Tumor recurrence as determined by flux >5E7 (p/s) are indicated for each treatment population: 10/10 untreated mice, 6/10 CAR-T+isotype control treated mice, and 3/10 CAR-T+anti-CD39 antibodies experienced tumor recurrence).

FIG. 5 depicts decreased overall tumor burden through IVIS imaging of a mouse xenograft model using luciferase expressing Raji tumor cells in groups treated with CAR-T in combination with anti-CD39 antibodies. Untreated mice were removed from study due to tumor burden following day 14, while mice in both CAR-T therapy treated groups were imaged over the complete study.

FIG. 6 depicts day 7 immune response of mice bearing Bi 6F10 melanoma tumors following treatment with adoptive cell therapy using melanoma antigen specific Pmel-1 CD8+ T cells in combination with anti-CD39 antibodies. (A) shows that proliferation of adoptively transferred antigen specific CD8+ T cells (top) and endogenous CD8+ T cells (bottom) at the site of antigen presentation in the lymph node is enhanced by treatment with anti-CD39 antibodies as measured by Ki67 staining. (B) shows improved early activation of adoptively transferred antigen specific CD8+ T cells (top) and endogenous CD8+ T cells (bottom) in the lymph node through treatment with anti-CD39 antibodies as measured by CD39 staining.

FIG. 7 depicts that at the termination of the study on day 35, mice that received treatment with anti-CD39 antibodies in combination with adoptive cell therapy with melanoma antigen specific Pmel-1 CD8+ T cells have decreased exhaustion of antigen specific CD8+ T cells and endogenous CD8+ T cells as measured by CD39 expression in the (A) lymph nodes (LN) and (B) spleen (SP).

### DETAILED DESCRIPTION

Provided herein are combination therapies involving antibodies with binding specificity for CD39 and adoptive cell therapy.

### Definitions

Unless otherwise defined, all terms of art, notations, and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the molecular cloning methodologies described in Sambrook et al., Molecular Cloning: A Laboratory Manual 4^{th} ed. (2014) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers.

The terms “CD39” and “Cluster of Differentiation 39” are used interchangeably herein.CD39 is also known as also known as ectonucleoside triphosphate diphosphohydrolase-1 (gene: ENTPDJ, protein: NTPD as el, see www.ncbi.nlm.nih.gov/gene/953). CD39 has also been referred to as ATPDase and SPG64. Each of the terms set forth above may be used interchangeably. Unless specified otherwise, the terms include any variants, isoforms, and species homologs of human CD39 that are naturally expressed by cells or that are expressed by cells transfected with a CD39 gene. In some embodiments, CD39 proteins include murine CD39. In some embodiments, CD39 proteins include cynomolgus CD39.

The term “immunoglobulin” refers to a class of structurally related proteins generally comprising two pairs of polypeptide chains: one pair of light (L) chains and one pair of heavy (H) chains. In an intact immunoglobulin all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (VH) and a heavy chain constant region (CH). The heavy chain constant region typically comprises three domains: CH1, CH2, and CH3. Each light chain typically comprises a light chain variable region (VL) and a light chain constant region. The light chain constant region typically comprises one domain, abbreviated CL.

The term “adoptive cell therapy” refers to immunotherapy in which immune cells are administered to a subject to help the subject fight diseases, such as cancer or a viral infection. In cancer therapy, for example, T cells are taken from a subject's own blood (or from a donor's blood) or tumor tissue, grown in large numbers, and then given back to the subject to help the subject fight cancer. Types of adoptive cell therapy include tumor-infiltrating lymphocyte (“TIL”) therapy, T-cell receptor (“TCR”) therapy, and chimeric antigen receptor T-cell (CAR-T-cell) therapy.

“Tumor-infiltrating lymphocyte (“TIL”) therapy” or “TIL therapy” refers to an immunotherapy that is an adoptive cell therapy that uses lymphocytes that are in or near a tumor and have an ability to recognize the tumor. In TIL therapy, lymphocytes, such as T-cells, that are in or near a tumor are isolated and then treated with substances that makes them grow to large numbers quickly. Those lymphocytes are then given back to the subject.

“T-cell receptor therapy” or “TCR therapy” refers to a type of adoptive cell therapy that involves engineering a subject's or donor's T or immune cells to express a particular or specific T-cell receptor or TCR.

“Chimeric antigen receptor T cell therapy” or “CAR-T therapy” refers to an adoptive cell therapy where one or more parts of a T cell receptor is changed into an extracellular binding moiety, such as an antibody or antibody fragment. The extracellular binding moiety, such as the antibody or antibody fragment, can be targeted to a tumor associated antigen (TAA) or a tumor specific antigen (TSA).

The term “antibody” describes a type of immunoglobulin molecule and is used herein in its broadest sense. An antibody specifically includes intact antibodies (e.g., intact immunoglobulins) and antibody fragments. Antibodies comprise at least one antigen-binding domain. One example of an antigen-binding domain is an antigen binding domain formed by a VH-VL dimer.

The VH and VL regions may be further subdivided into regions of hypervariability (“hypervariable regions (HVRs);” also called “complementarity determining regions” (CDRs)) interspersed with regions that are more conserved. The more conserved regions are called framework regions (FRs). Each VH and VL generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. See Kabat et al., Sequences ofProteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgGl, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J Mol. Biol., 273:927-948 (“Chothia” numbering scheme); MacCallum et al., 1996, J Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat numbering scheme. Variant and equivalent antibodies with a Chothia numbering scheme are intended to be within the scope of the invention.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L} depending on the orientation of the variable domains in the scFv (i.e., V_{H}-V_{L} or V_{L}-V_{H}). Any suitable Fc domain known in the art or described herein may be used.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies.

For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions with detection by Coomassie blue or silver stain. An isolated antibody includes an antibody in situ within recombinant cells, since at least one component of the antibody's natural environment is not present. In some embodiments, an isolated antibody is prepared by at least one purification step.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Affinity can be determined, for example, using surface plasmon resonance (SPR) technology, such as a Biacore® instrument.

With regard to the binding of an antibody to a target molecule, the terms “binding” or “binds to” a particular antigen (e.g., a polypeptide target) or an epitope on a particular antigen mean binding that is measurably different from a non-selective interaction. Binding can be measured, for example, by determining binding of a molecule compared to binding of a control molecule. Binding can also be determined by competition with a control molecule that is similar to the target, such as an excess of non-labeled target. In that case, binding is indicated if the binding of the labeled target to a probe is competitively inhibited by the excess non-labeled target.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, T, V, W, |
| | and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn | A, C, D, E, G, H, K, N, Q, R, S, |
| Formation | P, and T |
| Flexible Residues | Q, T, K, S, G, P, D, E, and R |
| Group 1 | A, S, and T |
| Group 2 | D and E |
| Group 3 | N and Q |
| Group 4 | R and K |
| Group 5 | I, L, and M |
| Group 6 | F, Y, and W |
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W.H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), Glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Antigen” refers to structures which may be specifically bound by antibodies or antibody fragments.

“TAA,” “tumor associated antigen,” or “tumor antigen” refer to an antigen produced in tumor cells. TAAs are useful tumor markers and candidates for use as antigens in cancer therapy.

“TSA,” or “tumor specific antigen” refer to antigens unique to tumor cells.

“Monocyte” refers to a type of white blood cell. Monocytes can differentiate into macrophages and myloid lineage dependent dendritic cells.

“Macrophages” refer to a type of white blood cell that engulfs and digests cellular debris, microbes, and cancer cells that do not have certain recognition proteins on their surface. The process of engulfing is called phagocytosis. Macrophages also play a critical role in innate immunity and help initiate adaptive immunity by recruiting other immune cells such as lymphocytes. Macrophages are important as antigen presenters to T cells. Macrophages can play a role in increasing and decreasing inflammation, with those that increase inflammation called M1 macrophages and those that decrease inflammation being called M2 macrophages.

“Dendritic cell” refers to antigen presenting cells of the immune system. Among other things, dendritic cells process antigen material and present it on the surface to T cells of the immune system. Dendritic cells play a role in the innate and adaptive immune systems. Once activated, dendritic cells migrate to lymph nodes where they interact with T cells and B cells to help shape the adaptive immune system.

“Lymphocyte” refers to a type of white blood cell in a vertabrate's immune system. Lymphocytes include natural killer cells, T cells, and B cells. Lymphocytes are the main type of cell found in lymph.

“NK cells,” “natural killer cells,” “K cells,” and “killer cells” refer to a lymphocyte which is a component of the innate immune system. NK cells play a major role in the host rejection of tumors and virally infected cells. NK cells are cytotoxic and small granules in their cytoplasm contain special proteins such as perforin and proteases known as granzymes. NK cells are often active in response to interferons or macrophage-derived cytokines.

“T cells” or “T lymphocytes” refer to a type of lymphocyte which develops in the thymus and plays a central role in immune response. T cells can be distinguished from other lymphocytes by the presence of a T cell receptor on the cell surface.

“TCR complex” refers to the TCR, G-chain, and CD3 molecules together.

“T cell receptor” or “TCR” refers to a molecule found on the surface of T cells that is responsible for recognizing fragments of antigen as peptides bound to major histocompatibility (MHC) complex molecules. The T cell receptor is a heterodimer consisting of two subunits, either TCRα and TCRβ or TCRγ and TCRδ. When a TCR engages with an antigen and an MHC, the T cell is activated through signal transduction.

The CD3 T cell co-receptor helps to activate both the cytotoxic T cell (CD8+ naive T cells) and also T helper cells (CD4+ naive T cells). The CD3 consists of a protein complex. In mammals, the complex contains a CD3γ chain, a CD36 chain, and two CD3ε chains. CD3 chains associate with the T cell receptor and the ζ-chain (zeta-chain) to generate an activation signal.

“ζ-chain” or “CD3ζ signaling domain” or “CD3ζ or “zeta-chain” or “T-cell surface recognition glycoprotein CD3 zeta chain” or “CD247” refers to a protein that plays an important role in coupling antigen recognition to intracellular signal transduction pathways.

“Natural killer T cells” or “NK T cells” refer to a heterogeneous group of T cells that share properties of both T cells and natural killer cells.

“B cells” or “B lymphocytes” refer to a type of white blood cell of the small lymphocyte subtype. They are active in the adaptive immune system by secreting antibodies. They also present antigen and secrete cytokines. Unlike T cells and NK cells, B cells express B cell receptors on their cell membrane.

“Peripheral blood mononuclear cell” or “PBMCs” refers to any peripheral blood cell having a round nucleus. Peripheral blood cells include, for example, lymphocytes and monocytes.

“Peripheral blood cell” refers to the cellular components of blood, consisting of red blood cells, such as erythrocytes, white blood cells, such as leucocytes, and platelets. Peripheral blood cells are found within the circulating poll of blood and not sequestered within the lymphatic system, spleen, liver, or bone marrow.

“Stimulation” or “stimulatory” refers to an event where binding of a molecule (i.e., a stimulatory molecule) mediates signal transduction. A stimulatory molecule is a molecule on an immune cell, such as a T cell, that binds a cognate stimulatory ligand or receptor present on an antigen presenting cell. When present on the antigen presenting cell (e.g., a dendritic cell, a B-cell, and the like), the stimulatory ligand or receptor can specifically bind with a stimulatory ligand or receptor on a T cell, thereby mediating a primary response by the T cell including, but not limited to, activation and initiation of an immune response. Stimulatory molecules include, but are not limited to, an MHC molecule loaded with a peptide, an anti-CD3 antibody, an anti-CD28 antibody, or an anti-CD2 antibody.

A “costimulatory signal” refers to a signal which, in combination with a stimulatory signal, leads to a T cell or antigen-presenting cell response such as proliferation, and upregulation or down regulation of an immune response.

A “costimulatory molecule” refers to a cognate binding partner on a T cell or antigen-presenting cell that binds with a costimulatory ligand or receptor and mediates a costimulatory response on a T cell or antigen-presenting cell such as proliferation. Costimulatory molecules include, but are not limited to, CD27, CD28, 4-1BB, GITR, OX40, CD30, CD40, CD83, ICOS, LFA-1, CD2, TNFSF14, NKG2C, and CD83.

A “costimulatory ligand” refers to a molecule, such as a molecule on T cell or antigen-presenting cell, that binds a cognate costimulatory receptor on a T cell or antigen-presenting cell. Binding of the costimulatory ligand provides a signal that mediates a T cell or antigen-presenting cell response, including proliferation, activation, or differentiation. A costimulatory introduces a signal that is in addition to a stimulatory signal. A costimulatory ligand may include, but not be limited to, CD7, B7-1 (CD80), B7-2 (CD86), 4-1BB ligand, OX40 ligand, inducible costimulatory ligand (ICOS-L), intercellular adhesion molecule (ICAM), CD30 ligand, CD40 ligand, CD70, CD83, lympho-toxin beta receptor, 3/TR6, immunoglobulin-like, ILT3, and an agonist or antibody that binds Toll ligand receptor.

“Treating” or “treatment” of any cancer refers, in certain embodiments, to ameliorating a cancer that exists in a subject. In another embodiment, “treating” or “treatment” includes ameliorating at least one physical parameter, which may be indiscernible by the subject. In yet another embodiment, “treating” or “treatment” includes modulating the cancer, either physically (e.g., stabilization of a discernible symptom) or physiologically (e.g., stabilization of a physical parameter) or both.

The terms “conditioning” and “pre-conditioning” refer to preparing a subject for adoptive cell therapy by, for example, giving the subject chemotherapy. Conditioning includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy.

As used herein, the term “subject” means a mammalian subject. Exemplary subjects include, but are not limited to humans, monkeys, dogs, cats, mice, rats, cows, horses, camels, avians, goats and sheep. In some embodiments, the subject is a human. In some embodiments, the subject has cancer, an autoimmune disease or condition, an inflammatory disease or condition, and/or an infection that can be treated with an antibody provided herein. In some embodiments, the subject is a human that is suspected to have cancer, an autoimmune disease or condition, and/or an acute infection and chronic infection.

As used herein, the term “therapeutically effective amount” or “effective amount” refers to an amount of an antibody and/or adoptive cell therapy that when administered to a subject is effective to treat a cancer. In some embodiments, a therapeutically effective amount comprises or consists of exemplary doses of each antibody and/or adoptive cell therapy. In some embodiments, a therapeutically effective amount comprises or consists of determining an amount used to achieve a response according to a clinical endpoint. In some embodiments, the clinical endpoint comprises Objective Response Rate (ORR), Progression Free Survival (PFS), and/or Response Evaluation Criteria in Solid Tumors (“RECIST”).

### Combinations

Provided herein are combinations of adoptive cell therapy and antibodies for the treatment of cancer.

A first aspect provides a therapeutic composition comprising an antibody which binds to CD39 and an adoptive cell therapy composition. In some embodiments, the adoptive cell therapy comprises or consists of TIL therapy, TCR therapy, and/or CAR therapy.

In some embodiments, the adoptive cell therapy composition comprises or consists of immune effector cells. In some embodiments, the immune effector cells comprise or consist of NK cells, T cells, NKT cells, dendritic cells, macrophages, peripheral blood mononuclear cells (PBMCs), αβ T cells, γδ T cells, regulatory T cells, NK T cells, or a combination thereof. In some embodiments, the adoptive cell therapy composition comprises or consists of immune effector cells comprising or consisting of engineered cells expressing a heterologous TCR and/or a CAR. In some embodiments, the engineered cells are T cells. In some embodiments, the T cells are CD8+ T cells.

In some embodiments, the engineered cells express a CAR comprising an extracellular binding moiety that specifically binds a tumor-associated antigen (TAA). In some embodiments, the TAA is a B cell antigen. In some embodiments, the CAR comprises an anti-CD19 binding moiety. In some embodiments, the TAA is a melanoma antigen. In some embodiments, the melanoma antigen is gp100.

In some embodiments, the CAR comprises an extracellular binding moiety, a transmembrane domain, and an intracellular domain that triggers the activation and/or proliferation of lymphocytes. In some embodiments, the intracellular domain comprises a CD3ζ signaling domain. In some embodiments, the CAR further comprises at least one costimulatory domain or a combination of costimulatory domains with other costimulatory domains and/or inhibitory domains. In some embodiments, the CAR comprises a CD28 or 4-1BB costimulatory domain.

In some embodiments, the engineered cells express the heterologous TCR. In some embodiments, the heterologous TCR specifically binds a TAA. In some embodiments, the TAA is a melanoma antigen. In some embodiments, TAA is gp100. In some embodiments, the heterologous TCR is a pmel-1 TCR.

In some embodiments, the antibody which binds to CD39 comprises a heavy chain variable region (VH) and a light chain variable region (VL), the VH and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

A second aspect provides a method of treating a subject in need thereof, the method comprising:

(a) administering to the subject an adoptive cell therapy composition; and

(b) simultaneously or sequentially administering to the subject an anti-CD39 antibody or immunologically active fragment thereof,

wherein the method comprises performing (a) before (b), performing (b) before (a), performing (a) and (b) simultaneously, or a combination thereof. In some embodiments, the method comprises administering the adoptive cell therapy composition and waiting for cells of the composition to migrate to a target cell or tissue of the subject before performing (b). In some embodiments, step (b) comprises administering the anti-CD39 antibody or immunologically active fragment thereof at a dose schedule.

In some embodiments, the dose schedule comprises administering the anti-CD39 antibody or immunologically active fragment thereof weekly, twice-monthly, monthly, or every two months. In some embodiments, the method further comprises at least one additional step of administering the adoptive cell therapy composition. In some embodiments, the adoptive cell therapy comprises or consists of TIL therapy, TCR therapy, and/or CAR therapy.

In some embodiments, the adoptive cell therapy composition comprises NK cells, T cells, dendritic cells, macrophages, PBMCs, aβ T cells, γδ T cells, regulatory T cells, NK T cells, or a combination thereof. In some embodiments, the adoptive cell therapy composition comprises engineered cells expressing a heterologous TCR and/or a CAR. In some embodiments, the engineered cells are T cells. In some embodiments, the T cells are CD8+ T cells.

In some embodiments, the engineered cells express a CAR comprising an extracellular binding moiety that specifically binds a TAA. In some embodiments, the TAA is a B cell antigen. In some embodiments, the CAR comprises an anti-CD19 binding moiety. In some embodiments, the TAA is a melanoma antigen. In some embodiments, the melanoma antigen is gp100.

In some embodiments, the CAR comprises the extracellular binding moiety, a transmembrane domain, and an intracellular domain that triggers the activation, persistence, survival, and/or proliferation of lymphocytes. In some embodiments, the intracellular domain comprises a CD3 signaling domain. In some embodiments, the CAR further comprises at least one costimulatory domain or a combination of costimulatory domains with other costimulatory domains and/or inhibitory domains. In some embodiments, the CAR comprises a CD28 or 4-1BB costimulatory domain.

In some embodiments, the engineered cells express the heterologous TCR. In some embodiments, the heterologous TCR specifically binds a TAA. In some embodiments, the TAA is a melanoma antigen. In some embodiments, the TAA is gp100. In some embodiments, the heterologous TCR is a pmel-1 TCR.

In some embodiments, the antibody which binds to CD39 comprises a heavy chain variable region (VH) and a light chain variable region (VL), the VH and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

A third aspect provides a method of producing an adoptive cell therapy composition, the method comprising:

(a) providing a first population of cells; and

(b) culturing the first population of cells under conditions sufficient to generate a therapeutically effective amount of cells for adoptive cell therapy in the presence of an anti-CD39 antibody or immunologically active fragment thereof. In some embodiments, the first population of cells comprises cells obtained from a subject in need of adoptive cell therapy treatment (or a donor). In some embodiments, the first population of cells comprises cells obtained from a donor subject and the adoptive cell therapy composition comprises cells produced for treatment of a recipient subject. In some embodiments, the adoptive cell therapy comprises or consists of TIL therapy, TCR therapy, and/or CAR therapy.

In some embodiments, the method further comprises engineering the first population of cells, or a portion thereof, and/or engineering a second population of cells, or a portion thereof, wherein the second population of cells are produced by expanding the first population of cells before or during step (b), wherein the engineering the first and/or second population of cells comprises genetically modifying the cells to express a CAR or a heterologous TCR.

In some embodiments, the anti-CD39 antibody comprises a heavy chain variable region (VH) and a light chain variable region (VL), the VH and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

### CDR-H1+CDR-H2+CDR-H3 Regions of the CD39 Antibodies

In some embodiments, the antibody that binds to CD39 comprises a V_{H} sequence comprising a CDR-H1 sequence comprising, consisting of, or consisting essentially of a sequence selected from SEQ ID NOs: 1-21 or SEQ ID NOs: 259-263, a CDR-H2 sequence comprising, consisting of, or consisting essentially of a sequence selected from SEQ ID NOs: 24-42 or SEQ ID NOs: 265-269, and a CDR-H3 sequence comprising, consisting of, or consisting essentially of a sequence selected from SEQ ID NOs: 45-72 or SEQ ID NOs: 271-275. In some embodiments, the CDR-H1 sequence, CDR-H2 sequence, and the CDR-H3 sequence are all from a single illustrative V_{H} sequence provided in this disclosure. For example, in some embodiments, the CDR-H1, CDR-H2, and CDR-H3 are all from a single illustrative V_{H} sequence selected from SEQ ID NOs: 138-177 or SEQ ID NO: 295, SEQ ID NO: 299, SEQ ID NO: 303, SEQ ID NO: 307, or SEQ ID NO: 311.

In some embodiments, the antibody that binds to CD39 comprises a V_{H} sequence comprising a CDR-H1 sequence comprising SEQ ID NO: 2, a CDR-H2 sequence comprising SEQ ID NO: 25, and a CDR-H3 sequence comprising SEQ ID NO: 46.

### V_{H} Sequences of the CD39 Antibody

In some embodiments, the antibody that binds CD39 comprises a V_{H} sequence comprising, consisting of, or consisting essentially of a sequence selected from SEQ ID NOs: 138-177 or SEQ ID NO: 295, SEQ ID NO: 299, SEQ ID NO: 303, SEQ ID NO: 307, or SEQ ID NO: 311. In some embodiments, the antibody that binds CD39 comprises a V_{H} sequence comprising, consisting of, or consisting essentially of SEQ ID NO: 139.

### CDR-L1+CDR-L2+CDR-L3 Regions of the CD39 Antibody

In some embodiments, the antibody which binds to CD39 comprises a V_{L} sequence comprising a CDR-L1 sequence comprising, consisting of, or consisting essentially of a sequence selected from SEQ ID NOs: 75-89 or SEQ ID NOs: 277-281, a CDR-L2 sequence comprising, consisting of, or consisting essentially of a sequence selected from SEQ ID NOs: 92-107 or SEQ ID NOs: 283-287, and a CDR-L3 sequence comprising, consisting of, or consisting essentially of a sequence selected from SEQ ID NOs: 110-135 or SEQ ID NOs: 289-293. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOs: 180-209 or SEQ ID NO: 296, SEQ ID NO: 300, SEQ ID NO: 304, SEQ ID NO: 308, or SEQ ID NO: 312. In some embodiments, the antibody which binds to CD39 comprises a VL sequence comprising a CDR-L1 sequence comprising SEQ ID NO: 76, a CDR-L2 sequence comprising SEQ ID NO: 93, and a CDR-L3 sequence comprising SEQ ID NO: 111.

### V_{L} Sequences of the CD39 Antibody

In some embodiments, the antibody that binds to CD39 comprises a V_{L} sequence comprising, consisting of, or consisting essentially of a sequence selected from any one of SEQ ID NOs: 180-209 or SEQ ID NO: 296, SEQ ID NO: 300, SEQ ID NO: 304, SEQ ID NO: 308, or SEQ ID NO: 312. In some embodiments, the antibody that binds to CD39 comprises a V_{L} sequence comprising, consisting of, or consisting essentially of SEQ ID NO: 181.

V_{H}-V_{L} Pairs for the CD39 Antibody

In some embodiments, the antibody which binds CD39 comprises a V_{H} sequence and a V_{L} sequence. In some embodiments, the V_{H} sequence is a V_{H} sequence comprising, consisting of, or consisting essentially of any one of SEQ ID NOs: 138-177 or SEQ ID NO: 295, SEQ ID NO: 299, SEQ ID NO: 303, SEQ ID NO: 307, or SEQ ID NO: 311 and the V_{L} sequence is a V_{L} sequence comprising, consisting of, or consisting essentially of any one of SEQ ID NOs: 180-209 or SEQ ID NO: 296, SEQ ID NO: 300, SEQ ID NO: 304, SEQ ID NO: 308, or SEQ ID NO: 312. In some embodiments, the antibody which binds to CD39 comprises a V_{H} sequence comprising SEQ ID NO: 139 and V_{L} sequence comprising SEQ ID NO: 181.

### CDR-H1+CDR-H2+CDR-H3+CDR-L1+CDR-L2+CDR-L3 of CD39 Antibodies

In some embodiments, the antibody which binds to CD39 comprises or consists of a heavy chain variable region (V_{H}) and a light chain variable region (VL), with the V_{H} and/or VL together comprising 1, 2, 3, 4, 5, or 6 of:

(a) a VHCDR1 having the sequence set forth in any one of SEQ ID NOs: 1-21 or SEQ ID NOs: 259-263,

(b) a VHCDR2 having the sequence set forth in any one of SEQ ID NOs: 24-42 or SEQ ID NOs: 265-269,

(c) a VHCDR3 having the sequence set forth in any one of SEQ ID NOs: 45-72 or SEQ ID NOs: 271-275,

(d) a VLCDR1 having the sequence set forth in any one of SEQ ID NOs: 75-89 or SEQ ID NOs: 277-281,

(e) a VLCDR2 having the sequence set forth in any one of SEQ ID NOs: 92-107 or SEQ ID NOs: 283-287, and

(f) a VLCDR3 having the sequence set forth in any one of SEQ ID NOs: 110-135 or SEQ ID NOs: 289-293.

In some embodiments, the antibody which binds to CD39 comprises a V_{H} sequence comprising a CDR-H1 sequence comprising SEQ ID NO: 2, a CDR-H2 sequence comprising SEQ ID NO: 25, and a CDR-H3 sequence comprising SEQ ID NO: 46 and a VL sequence comprising a CDR-L1 sequence comprising SEQ ID NO: 76, a CDR-L2 sequence comprising SEQ ID NO: 93, and a CDR-L3 sequence SEQ ID NO: 111.

### HC+LC

In some embodiments, the antibody that binds CD39 comprises or consists of one or more heavy chains consisting of an HC sequence and one or more light chains consisting of an LC sequence. In some embodiments, the antibody that binds CD39 comprises or consists of two identical heavy chains consisting of an HC sequence and two identical light chains consisting of an LC sequence.

In some embodiments, the HC sequence of the antibody that binds CD39 is an HC sequence comprising, consisting of, or consisting essentially of any one of SEQ ID NOs: 212, 214, 216, 218, 220, 222, 224, 226, 228, 230, 232, 234, 236, 238, 240, 242, 244, 246, 248, 250, 252, 254, 297, 301, 305, 309, 313 and the LC sequence of the antibody that binds CD39 is an LC sequence comprising, consisting of, or consisting essentially of any one of SEQ ID NOs: 213, 215, 217, 219, 221, 223, 225, 227, 229, 231, 233, 235, 237, 239, 241, 243, 245, 247, 249, 251, 253, 255, 298, 302, 306, 310, 314. In some embodiments, the HC sequence of the antibody that binds CD39 is an HC sequence consisting of SEQ ID NO: 212 and the LC sequence is an LC sequence consisting of SEQ ID NO: 213.

### Glycosylation Variants of the CD39 Antibodies

In certain embodiments, an antibody of the invention may be altered to increase, decrease, or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In certain embodiments, the antibody is glycosylated. In certain embodiments, the antibody is deglycosylated. Carbohydrates may be removed by standard techniques. In certain embodiments, the antibody is aglycosylated, for instance by expression in a system that does not glycosylate.

### Preparation of CD39 Antibodies

### Antigen Preparation

CD39 antigens used for production of antibodies may be intact CD39 or a fragment of CD39. The intact CD39, or fragment of CD39, may be in the form of an isolated protein or may be expressed by a cell. One skilled in the art will appreciate how to prepare CD39 antigen whether it be intact or a fragment. Antigens may be prepared as set forth in WO/2019/027935, which is incorporated by reference in its entirety herein. Other forms of CD39 useful for generating antibodies will be apparent to those skilled in the art.

### CD39 Monoclonal Antibodies

In some embodiments, the antibodies that bind CD39 are monoclonal antibodies. Monoclonal antibodies may be obtained, for example, using the hybridoma method first described by Kohler et al., Nature, 1975, 256:495-497, and/or by recombinant DNA methods (see e.g., U.S. Pat. No. 4,816,567). Monoclonal antibodies may also be obtained, for example, using phage or yeast-based libraries. See e.g., U.S. Pat. Nos. 8,258,082 and 8,691,730.

In the hybridoma method, a mouse or other appropriate host animal is immunized to elicit lymphocytes that produce or are capable of producing antibodies that will specifically bind to the protein used for immunization. Alternatively, lymphocytes may be immunized in-vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, Calif.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in-vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### CD39 Humanized Antibodies

Humanized antibodies may be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. USA., 1998, 95:8910-8915; Steinberger et al., J Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370.

In some embodiments, the antibody that binds CD39 is B66. In some embodiments, the antibody that binds CD39 is a humanized anti-CD39 comprising B66 (See, for example, WO/2019/027935, which is incorporated by reference in its entirety herein).

### CD39 Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807. Human antibodies can also be derived from phage-display libraries (see e.g., Hoogenboom et al., J. Mol. Biol., 1991, 227:381-388; Marks et al., J Mol. Biol., 1991, 222:581-597; and U.S. Pat. Nos. 5,565,332 and 5,573,905). Human antibodies may also be generated by in-vitro activated B cells (see e.g., U.S. Pat. Nos. 5,567,610 and 5,229,275). Human antibodies may also be derived from yeast-based libraries (see e.g., U.S. Pat. No. 8,691,730).

In some embodiments, the antibody that binds CD39 is a human antibody (See, for example, WO/2019/027935, which is incorporated by reference in its entirety herein).

### Extraction for Adoptive Cell Therapy

Adoptive cell therapy uses a subject's immune effector cells, a donor's immune effector cells, or a cell line comprising immune effector cells that may intrinsically or be engineered to attack a subject's cancer. Accordingly, a subject's or donor's white blood cells, including T cells, must initially be separated from the rest of a subject sample. In some embodiments, extraction comprises or consists of isolating T cells. In some embodiments, isolated T cells comprise or consist of CD4+ T cells and/or CD8+ T cells. One skilled in the art would understand how to extract patient T cells (See, for example, Molecular Therapy Oncolytics, Volume 3, 2016, 16015, which is incorporated by reference in its entirety herein).

In some embodiments, the subject or donor sample comprises or consists of whole blood. In some embodiments, the subject or donor sample is centrifuged. In some embodiments, the subject or donor sample comprises or consists of a buffy coat sample. In some embodiments, the subject or donor sample comprises or consists of a PBMC sample. In some embodiments, the subject or donor sample comprises or consists of an unfractionated T cell sample. In some embodiments, the subject or donor sample comprises or consist of a lymphocyte sample. In some embodiments, the subject or donor sample comprises or consists of a white blood cell sample. In some embodiments, the subject or donor sample comprises or consists of an apheresis sample. In some embodiments, the subject or donor sample comprises or consists of a leukaphersis sample.

In some embodiments, the adoptive cell therapy composition comprises NK cells, T cells, dendritic cells, macrophages, peripheral blood mononuclear cells (PBMCs), αβ T cells, γδ T cells, regulatory T cells, NK T cells, or a combination thereof. In some embodiments, the adoptive cell therapy composition comprises engineered cells expressing a heterologous TCR and/or CAR. In some embodiments, the engineered cells are T cells. In some embodiments, the T cells are CD8+ T cells.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors. T cells can be obtained from a unit of blood collected from a subject or donor using any number of techniques known to the skilled artisan, such as Ficoll™ separation. In some embodiments, T cells from the circulating blood of a subject or donor are obtained by apheresis. The apheresis product typically contains lymphocytes, including T cells, monocytes, granulocytes, B cells, other nucleated white blood cells, red blood cells, and platelets.

In some embodiments, T cells collected by apheresis may be washed to remove a plasma fraction. In some embodiments, T cells are washed with a wash solution. In some embodiments, the wash solution comprises or consists of a phosphate buffered saline (PBS). In some embodiments, the wash solution lacks calcium, magnesium, and/or selected divalent cations. One skilled in the art will appreciate that washing may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions.

In some embodiments, T cells are placed in an appropriate buffer, culture, or media for subsequent processing steps. For example, cells may be resuspended in a variety of biocompatible buffers, such as, for example, Ca-free, Mg-free PBS, PlasmaLyte A, or other saline solution with or without buffer. In some embodiments, media conditions comprise or consist of 5% or less human AB serum, and employ known culture media conditions and compositions (See, Smith et al., Ex-vivo expansion of human T cells for adoptive immunotherapy using the novel Xeno-free CTS Immune Cell Serum Replacement” Clinical & Translational Immunology (2015) 4, e31; doi:10.1038/cti.2014.31, which is incorporated by reference herein in its entirety).

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45R0+ T cells can be further isolated by positive or negative selection techniques. In some embodiments, T cells are isolated by incubation with anti-CD3/anti-CD28 (e.g., 3×28)-conjugated beads, such as DYNABEADS® M450 CD3/CD28 T, for a time period sufficient for positive selection of the desired T cells.

In some embodiments, the time period is about 30 minutes. In some embodiments, the time period ranges from 30 minutes to 36 hours or longer. In some embodiments, the time period is at least 1, 2, 3, 4, 5, or 6 hours. In some embodiments, the time period is 10 to 24 hours. In some embodiments, the time period is 24 hours.

Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to CD3/CD28 beads, such as CD3/CD28 beads, and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on beads or another surface, subpopulations of T cells can be preferentially selected for or against at culture initiation or at other desired time points. Multiple rounds of selection can also be used.

Enrichment of a T cell population by negative selection can be accomplished with a combination of antibodies directed to surface markers unique to negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail to CD14, CD20, CD11 b, CD16, HLA-DR, and CD8 may be used.

In some embodiments, it may be desirable to enrich for or negatively or positively select for regulatory T cells which typically express CD4+, CD25+, CD62Lhi, GITR+, and FoxP3+. Alternatively, in some embodiments, T regulatory cells may be depleted by anti-CD25 conjugated beads or other similar method of selection. In some embodiments, an enriched T cell population contains less than about 40%, about 35%, about 30%, about 25%, about 20%, about 15%, about 10%, about 5%, about 4%, about 3%, about 2%, or about 1% of CD25+ cells.

In some embodiments, T regulatory cells, e.g., CD25+ T cells, are removed from the population using an anti-CD25 antibody, or fragment thereof, or a CD25-binding ligand, such as, for example without limitation, IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the T regulatory cells, e.g., CD25+ T cells, are removed from the population using CD25 depletion reagent from Miltenyi™.

In some embodiments, a subject is pre-treated with one or more therapies that reduce regulatory T cells prior to collection of T cells. Pre-treating the subject may decrease the chance of relapse. In some embodiments, one or more of a cyclophosphamide, anti-GITR antibody, CD25-depletion, or a combination thereof is administered to the subject prior to isolation of T cells. Administration of one or more of cyclophosphamide, anti-GITR antibody, CD25-depletion, or a combination thereof, can ultimately occur before, during, or after an infusion of the CAR-expressing cell product.

In some embodiments, a population of cells is removed that are neither the regulatory T cells or tumor cells but cells that would otherwise negatively affect the expansion and/or function of cells (e.g. cells expressing CD14, CD11b, CD33, CD15, CD52, or other markers expressed by potentially immune suppressive cells). In some embodiments, such cells are removed concurrently with regulatory T cells and/or tumor cells, or following said depletion, or in another order. One or more than one selection step, e.g., more than one depletion step, may be included and the steps may occur in any order.

In some embodiments, T cells may be enriched by removing cells from the population which express a tumor antigen, e.g., a tumor antigen that does not comprise CD25, e.g., CD19, CD30, CD38, CD123, CD20, CD14, or CD11b, to thereby provide a population of T regulatory depleted, e.g., CD25+ depleted, and tumor antigen depleted cells. In some embodiments, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In some embodiments, the removal of T regulatory cells, e.g., CD25+ cells, and the removal of the tumor antigen expressing cells is sequential, and can occur, e.g., in either order.

T cells may also be frozen after washing. A freeze and subsequent thaw step may provide a more uniform product by removing granulocytes and/or monocytes in the cell population. After the washing step that removes plasma and platelets, the cells may be suspended in a freezing solution. In some embodiments, cryopreserved cells are thawed and washed and allowed to rest for one hour at room temperature prior to activation.

In some embodiments, T cells are obtained from a subject directly following treatment that leaves the subject with functional T cells. Following certain cancer treatments, in particular treatments with drugs that damage the immune system, shortly after treatment during the period when subjects would normally be recovering from the treatment, the quality of T cells obtained may be optimal or improved for their ability to expand ex-vivo.

### Reprogramming for Adoptive Cell Therapy

In some embodiments, one or more genes encoding one or more TCRs and/or one or more CARs is delivered to isolated immune cells to produce engineered cells In some embodiments, a viral vector is used to deliver the gene encoding the one or more TCRs and/or one or more CARs. In some embodiments, the vector comprises or consists of a DNA, RNA, a plasmid, an adenoviral vector, a lentivirus vector, or a retrovirus vector. In some embodiments, the viral vector comprises or consists of a retroviral vector. In some embodiments, the retroviral vector comprises or consists of a lentiviral vector. In some embodiments, the retroviral vector comprises or consists of a gammaretroviral vector.

In some embodiments, the isolated immune cells are delivered one or more genes that enhance the efficacy and/or safety of the engineered cells. In some embodiments, the one or more genes that enhance the efficacy of the engineered cells is one or more cytokines. In some embodiments, the one or more cytokine comprises or consists of IL-2, IL-7, IL-12, IL-15, and/or IL-18. In some embodiments, the one or more genes that enhance the efficacy of the engineered cells comprises or consists of a receptor. In some embodiments, the receptor comprises or consist of a chemokine receptor. In some embodiments, the chemokine receptor comprises or consists of CXCR2 and or CCR2B. In some embodiments, the receptor comprises or consists of a dominant negative receptor. In some embodiments, the dominant negative receptor comprises or consists of TGFBR2 (See, e.g., Depil et al. 2020 Nature Reviews Drug Discovery 19: 185-199, incorporated herein by reference in its entirety).

In some embodiments, the isolated immune cells are delivered one gene or more that enhances the safety of the engineered cells. In some embodiments, the gene that enhances the safety of the engineered cells comprises or consists of a safety switch. In some embodiments, the safety switch comprises or consists of a suicide switch. In some embodiments, the suicide switch comprises or consists of HSV-TK, iCasp9, CD20, and/or EGFRt. In some embodiments the safety switch comprises or consists of a system which controls the expression of the TCR and/or CAR gene. In some embodiments, the system controlling the expression of the TCR and or CAR gene comprises or consists of a synNotch system or inducible CAR system (See, e.g., Yu et al. 2019 Molecular Cancer 18: 125-138, incorporated herein by reference in its entirety).

Vectors derived from retroviruses, such as the lentivirus, are suitable tools to achieve long-term gene transfer since they allow long-term stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

A retroviral vector may also be, e.g., a gammaretroviral vector. A gammaretroviral vector may include, e.g., a promoter, a packaging signal, a primer binding site (PBS), one or more (e.g., two) long terminal repeats (LTR), and a transgene of interest, e.g., a TCR or CAR. A gammaretroviral vector may lack viral structural genes such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al. “Gammaretroviral Vectors: Biology, Technology and Application” Viruses. 2011 June; 3(6): 677-713.

In some embodiments incorporation of one or more TCR and/or CAR and/or delivery of one or more genes that enhances efficacy and/or survival occurs via gene editing. In some embodiments, genes are co-transferred or transferred sequentially with gene editing moieties. In some embodiments, the gene editing moieties comprise or consist of sleeping beauty, CRISPR, CAS9, TALEN, megaTAL, and zinc finger nucleases. (See, e.g., June et al. 2009 Nature Reviews Immunology 9.10: 704-716, incorporated herein by reference in its entirety).

The expression of a TCR and/or CAR is typically achieved by operably linking a nucleic acid encoding the TCR and/or CAR polypeptide or portions thereof to a promoter and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence

In some embodiments, the expression of a TCR and/or CAR is achieved by incorporating a TCR and/or CAR construct into a specific locus via gene editing. In some embodiments, this locus is the TRAC or CD52 locus (See, for example, Depil, et al., 2020, Cancer Cell Therapy, 19:185-199; Fisher, et al., 2018, Frontiers in Immunology, 9:1409; Wang, et al., 2020, Cancer Letters, 472:175-180; WO2017019848, which is incorporated by reference herein in its entirety).

An example of a promoter that is capable of expressing a CAR in a mammalian T cell is the EFlalpha promoter (EFla or EFla). The native EFla promoter drives expression of the alpha subunit of the elongation factor-1 complex, which is responsible for the enzymatic delivery of aminoacyl tRNAs to the ribosome. The EFla promoter has been extensively used in mammalian expression plasmids and has been shown to be effective in driving CAR expression from transgenes cloned into a lentiviral vector. (See, e.g., Milone et al., Mol. Ther. 17(8): 14531464 (2009), which is incorporated by reference in its entirety herein). In some embodiments, the vector comprises or consists of an EFlalpha promoter.

In some embodiments, the promoter comprises or consists of an immediate early cytomegalovirus (CMV) promoter, which is a strong constitutive promoter sequence capable of driving high levels of expression of any polynucleotide sequence operatively linked thereto. Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myoson promoter, the elongation factor-Ia promoter, the hemoglobin promoter, and the creatine kinase promoter.

In some embodiments, the promoter is an inducible promoter. The use of an inducible promoter provides a molecular switch capable of turning on expression of the polynucleotide sequence to which it is operatively linked when such expression is desired, or turning off the expression when expression is not desired. Examples of inducible promoters include, but are not limited to, a metallothionine promoter, a glucocorticoid promoter, a progesterone promoter, and a tetracycline promoter.

In some embodiments, the promoter is an endogenous promotor. The use of an endogenous promoter allows expression to be regulated in a manner that mimics the natural response to an antigen and prevents excessive cell stimulation that may lead to exhaustion.

In some embodiments, the vector comprises or consists of a signal sequence to facilitate secretion, a polyadenylation signal and a transcription terminator (e.g., from Bovine Growth Hormone (BGH) gene), an element allowing episomal replication and replication in prokaryotes (e.g. SV40 origin and ColE1 or others known in the art), and/or elements to allow selection (e.g., ampicillin resistance gene and/or zeocin marker).

Vectors may be introduced into T cells by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). Vectors may also be introduced into T cells by lipofection. Biological methods for introducing a polynucleotide of interest into a host cell include the use of DNA and RNA vectors.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in-vitro and in-vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Other viral vectors can be derived from lentivirus, poxviruses, herpes simplex virus I, adenoviruses and adeno-associated viruses, and the like (See, for example, U.S. Pat. Nos. 5,350,674 and 5,585,362, which is incorporated by reference in its entirety herein).

### Expanding Engineered Cells in Adoptive Cell Therapy

Cells for adoptive cell therapy may be expanded or multiplied. Immune effector cells, such as T cells, may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, each of which is incorporated by reference in its entirety herein. Generally, a population of immune effector cells, e.g., T cells may be expanded by contact with a surface having attached thereto an agent that stimulates a CD3/TCR complex associated signal and a ligand that stimulates a costimulatory molecule on the surface of the immune effector cells, e.g., T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells.

To stimulate proliferation of either CD4+ T cells or CD8+ T cells, an anti-CD3 antibody and an anti-CD28 antibody may be used as well as other methods commonly known in the art (See, Berg et al., Transplant Proc. 30(8):3975-3977, 1998; Haanen et al., J. Exp. Med. 190(9):13191328, 1999; Garland et al., J. Immunol Meth. 227(1-2):53-63, 1999, each of which is incorporated by reference herein, including any drawings). Examples of anti-CD28 antibodies include, without limitation, 9.3, B-T3, XR-CD28 (Diaclone, Besancon, France).

In some embodiments, a primary stimulatory signal and a costimulatory signal for the T cells may be provided for a period of time. In some embodiments, T cells are expanded in culture for a period of several hours (e.g., about 2, 3, 4, 5, 6, 7, 8, 9, 10, 11, 12, 13, 14, 15, 16, 17, 18, 19, 20, 21, 22, 23, or 24 hours) to about 14 days (e.g., 1, 2, 3, 4, 5, 6, 7, 8, 9, 10, 11, 12, 13, or 14 days). In some embodiments, T cells are expanded for a period of about 4 days to about 9 days. In some embodiments, T cells are expanded for a period of about 8 days or less. In some embodiments, T cells are expanded for about 7, about 6, or about 5 days.

In some embodiments, T cells are expanded in culture for 5 days and the resulting T cells exhibit higher proinflammatory cytokine production as compared to cells not expanded in culture. In some embodiments, proinflammatory cytokines comprises or consist of IFN-γ and/or GM-CSF.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFP, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and/or X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Target cells may be maintained under conditions necessary to support growth, for example, an appropriate temperature (e.g., 37° C.) and atmosphere (e.g., air plus 5% CO2).

In some embodiments, T cells are expanded in an appropriate media (e.g., media described herein) that includes one or more interleukins that result in at least a 200-fold (e.g., 200-fold, 250-fold, 300-fold, or 350-fold) increase in cells over a 14 day expansion period, e.g., as measured by a method such as flow cytometry. In some embodiments, the cells are expanded in the presence of IL-15 and/or IL-7 (e.g., IL-15 and IL-7).

In some embodiments, T regulatory cells, e.g., CD25+ T cells, are removed. In some embodiments, T regulatory cells are removed using an anti-CD25 antibody, or fragment thereof, or a CD25-binding ligand. Some embodiments further comprise contacting a cell population (e.g., a cell population in which T regulatory cells, such as CD25+ T cells, have been depleted; or a cell population that has previously contacted with an anti-CD25 antibody, fragment thereof, or CD25-binding ligand) with IL-15 and/or IL-7.

In some embodiments, the cells are cultured (e.g., expanded, simulated, and/or transduced) in media comprising serum. The serum may be, e.g., human AB serum (hAB). In some embodiments, the hAB serum is present at about 2%, about 5%, about 2-3%, about 3-4%, about 4-5%, or about 2-5%. (See, Smith et al., “Ex-vivo expansion of human T cells for adoptive immunotherapy using the novel Xeno-free CTS Immune Cell Serum Replacement” Clinical & Translational Immunology (2015) 4, e31; doi:10.1038/cti.2014.31, which is incorporated by reference in its entirety herein).

In some embodiments, engineered cells are expanded or multiplied in a bioreactor. In some embodiments, engineered cells are expanded, multiplied, or cultured in the presence of an anti-CD39 antibody or immunologically active fragment thereof.

Conditioning for Adoptive cell Therapy

In some embodiments, a subject may be conditioned prior to receiving adoptive cell therapy. As one example, the subject may receive chemotherapy to lower their white blood cell numbers to make room for engineered cells, such as CAR-T cells. As another example, a subject may receive chemotherapy to drive tumor cell death and/or promote release of ATP and homing of CD39 cells to a tumor site. Any therapy can be administered to a subject according to one skilled in the art.

In particular, bendamustine, ibrutinib, cyclophosphamide, alemtuzumab, and/or fludarabine may be administered to the subject as conditioning for adoptive cell therapy in some embodiments (See, for example, U.S. Pat. No. 9,855,298, which is incorporated by reference in its entirety herein). In some embodiments, conditioning comprises or consists of administering bendamustine, ibrutinib, cyclophosphamide, alemtuzumab, and/or fludarabine prior to administration of adoptive cell therapy.

In some embodiments, conditioning is administered to the subject 1, 2, 3, 4, 5, 6, 7, 8, 9, or 10 days prior to administration of the adoptive cell therapy. In some embodiments, conditioning is administered at a dose of about 100 mg/kg, about 125 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, or about 500 mg/kg. In some embodiments, conditioning comprises or consists of administering cyclophosphamide prior to administration of adoptive cell therapy. In some embodiments, conditioning comprises or consists of administering cyclophosphamide prior to administration of adoptive cell therapy at the dose and times provided herein.

In some embodiments, conditioning comprises or consists of increasing a serum level of one or more cytokines, e.g., interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof. In some embodiments, conditioning comprises increasing a serum level of IL-7, IL-15, IP-10, MCP-1, PLGF, CRP, or any combination thereof. In some embodiments, the serum level of IL-7, IL-15, IP-10, MCP-1, PLGF, CRP, or any combination thereof, is increased through the expression of one or more cytokines by the engineered cells comprising the TIL, TCR, and or CAR therapy.

### Administration of Therapy

Administration of the compositions described herein may be carried out in any convenient manner, including by aerosol inhalation, injection, ingestion, transfusion, implantation, or transplantation. The compositions described herein may be administered to a subject transarterially, subcutaneously, intradermally, intratumorally, intranodally, intramedullary, intramuscularly, by intravenous (i.v.) injection, or intraperitoneally.

In some embodiments, the administration of the composition comprises or consists of intradermal or subcutaneous injection. In some embodiments, administration of the composition comprises or consists of injection. In some embodiments, administration of the composition comprises or consists of injection directly into a tumor, lymph node, and/or site of infection.

A therapeutically effective of the antibody which binds CD39 and therapeutically effective amount of adoptive cell therapy may be determined by a physician with consideration of individual differences in age, weight, tumor size, extent of infection, extent of the cancer or metastasis, and condition of the subject. The dosage to be administered to a subject will vary depending upon the precise nature of the condition being treated and the subject. One skilled in the art would be able to determine the scaling of dosages for human administration. Effective doses may be extrapolated from dose-response curves derived from in-vitro or animal models.

For example, a therapeutically effective amount of the antibody which binds to CD39 comprises or consists of milligram or microgram amounts of the antibody per kilogram of subject or sample weight. In some embodiments, the dosage of the antibody is 0.1 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or 15 mg/kg or more of a subject's body weight. In some embodiments, the dosage of the antibody is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg. In some embodiments, the dosage of the antibody is 0.1 mg to 0.2 mg, 0.2 mg to 0.3 mg, 0.3 mg to 0.4 mg, 0.4 mg to 0.5, 0.5 mg to 0.6 mg, 0.6 mg to 0.7, 0.7 mg to 0.8 mg, or 0.8 mg to 0.9 mg.

The therapeutic amount of the adoptive cell therapy may also be determined according to one skilled in the art. In some embodiments, the adoptive cell therapy comprises or consists of a dosage of between about 10⁴ cells/kg body weight and about 10⁹ cells/kg body weight. In some embodiments, the adoptive cell therapy comprises or consists of a dosage between about 10⁵ cells/kg body weight and about 10⁶ cells/kg body weight. In some embodiments, the adoptive cell therapy comprises or consists of a dosage of integer value between about 10⁴ cells/kg body weight and about 10⁹ cells/kg body weight.

In some embodiments, the adoptive cell therapy comprises or consists of CAR therapy or CAR-T therapy. In some embodiments, the CAR therapy or CAR-T therapy comprises or consists of dosage of about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, or about 5×10⁸ cells/kg. In some embodiments, the CAR therapy or CAR-T therapy comprises or consists of dosage of about up to about 1×10⁶, up to about 2×10⁶, up to about 3×10⁶, up to about 4×10⁶, up to about 5×10⁶, up to about 6×10⁶, up to about 7×10⁶, up to about 8×10⁶, up to about 9×10⁶, up to about 1×10⁷, up to about 2×10⁷, up to about 3×10⁷, up to about 4×10⁷, up to about 5×10⁷, up to about 6×10⁷, up to about 7×10⁷, up to about 8×10⁷, up to about 9×10⁷, up to about 1×10⁸, up to about 2×10⁸, up to about 3×10⁸, up to about 4×10⁸, or up to about 5×10⁸ cells/kg.

In some embodiments, the CAR therapy or CAR-T therapy comprises or consists of dosage of about 1×10⁵ cells, about 2×10⁵ cells, about 3×10⁵ cells, about 4×10⁵ cells, about 5×10⁵ cells, about 6×10⁵ cells, about 7×10⁵ cells, about 8×10⁵ cells, about 9×10⁵ cells, about 1×10⁶ cells, about 2×10⁶ cells, about 3×10⁶ cells, about 4×10⁶ cells, about 5×10⁶ cells, about 6×10⁶ cells, about 7×10⁶ cells, about 8×10⁶ cells, about 9×10⁶ cells, about 1×10⁷ cells, about 2×10⁷ cells, about 3×10⁷ cells, about 4 ×10⁷ cells, about 5×10⁷ cells, about 6×10⁷ cells, about 7×10⁷ cells, about 8×10⁷ cells, about 9×10⁷ cells, about 1×10⁸ cells, about 2×10⁸ cells, about 3×10⁸ cells, about 4×10⁸ cells, about 5×10⁸ cells, about 6×10⁸ cells, about 7×10⁸ cells, about 8×10⁸ cells, about 9×10⁸ cells, about 1×10⁹ cells, about 2 ×10⁹ cells, about 3×10⁹ cells, about 4×10⁹ cells, or about 5×10⁹ cells. In some embodiments, the CAR therapy or CAR-T therapy comprises or consists of dosage of at least about 1×10⁵ cells, at least about 2×10⁵ cells, at least about 3×10⁵ cells, at least about 4×10⁵ cells, at least about 5×10⁵ cells, at least about 6 ×10⁵ cells, at least about 7×10⁵ cells, at least about 8×10⁵, cells, at least about 9×10⁵ cells, at least about 1×10⁶ cells, at least about 2×10⁶ cells, at least about 3×10⁶ cells, at least about 4×10⁶ cells, at least about 5×10⁶ cells, at least about 6×10⁶ cells, at least about 7×10⁶ cells, at least about 8×10⁶ cells, at least about 9×10⁶, at least about 1×10⁷ cells, at least about 2×10⁷ cells, at least about 3×10⁷ cells, at least about 4×10⁷ cells, at least about 5×10⁷ cells, at least about 6×10⁷ cells, at least about 7×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 7×10⁸ cells, at least about 8×10⁸ cells, at least about 9×10⁸ cells, at least about 1 ×10⁹ cells, at least about 2×10⁹ cells, at least about 3×10⁹ cells, at least about 4×10⁹ cells, or at least about 5×10⁹ cells.

In some embodiments, the CAR therapy or CAR-T therapy comprises or consists of dosage of up to about 1×10⁵ cells, up to about 2×10⁵ cells, up to about 3×10⁵ cells, up to about 4×10⁵ cells, up to about 5×10⁵ cells, up to about 6×10⁵ cells, up to about 7×10⁵ cells, up to about 8×10⁵ cells, up to about 9×10⁵ cells, up to about 1×10⁶ cells, up to about 2×10⁶ cells, up to about 3×10⁶ cells, up to about 4×10⁶ cells, up to about 5×10⁶ cells, up to about 6×10⁶ cells, up to about 7×10⁶ cells, up to about 8×10⁶ cells, up to about 9×10⁶ cells, up to about 1×10⁷ cells, up to about 2×10⁷ cells, up to about 3×10⁷ cells, up to about 4×10⁷ cells, up to about 5×10⁷ cells, up to about 6×10⁷ cells, up to about 7×10⁷ cells, up to about 8×10⁷ cells, up to about 9×10⁷ cells, up to about 1×10⁸ cells, up to about 2×10⁸ cells, up to about 3×10⁸ cells, up to about 4×10⁸ cells, up to about 5×10⁸ cells, up to about 6×10⁸ cells, up to about 7×10⁸ cells, up to about 8×10⁸ cells, up to about 9×10⁸ cells, up to about 1×10⁹ cells, up to about 2×10⁹ cells, up to about 3×10⁹ cells, up to about 4×10⁹ cells, or up to about 5×10⁹ cells.

In some embodiments, a therapeutically effective comprises or consists of exemplary doses of each antibody and/or adoptive cell therapy. In some embodiments, a therapeutically effective amount comprises or consists of determining an amount used to achieve a response according to a clinical endpoint. In some embodiments, the clinical endpoint comprises Objective Response Rate (ORR), Progression Free Survival (PFS), and/or Response Evaluation Criteria in Solid Tumors (“RECIST”).

Frequency of administration will be related to dosage and will also vary according to factors specific for each subject depending on the specific therapy administered, the severity of the disorder, disease, or condition, the route of administration, as well as age, body, weight, response, and the past medical history of the subject. Frequency may also be extrapolated from dose-response curves derived from in-vitro or animal models.

The antibody that binds CD39 may be administered according to a suitable schedule, for example, once, two times, three times, or four times weekly. The clinician or treating physician will know how and when to interrupt, adjust, or terminate therapy in conjunction with subject response. In some embodiments, the antibody that binds CD39 may be administered and administration may be repeated and one or more rounds of administration may separate by at least 1 day, 2 days, 3 days, 4 days, 5 days, 6 days, 7 days, 8 days, 9 days, 10 days, 15 days, 30 days, 45 days, 2 months, 75 days, 3 months, or 6 months.

The adoptive cell therapy may also be administered according to a suitable schedule. The antibody which binds to CD39 and/or adoptive cell therapy compositions may be administered multiple times and at the same of different dosages. The antibody which binds to CD39 and/or therapeutically effective amount of an adoptive cell therapy compositions may be administered in any order and at any dose. Some embodiments comprise or consist of administering to the subject an adoptive cell therapy composition and simultaneously or sequentially administering an anti-CD39 antibody or immunologically active fragment thereof. In some embodiments, the antibody which binds to CD39 is administered before the adoptive cell therapy. In some embodiments, the antibody which binds to CD39 is administered after the adoptive cell therapy. In some embodiments, the antibody which binds CD39 and/or the adoptive cell therapy are administered multiple times and at the same of different dosages, or a combination thereof

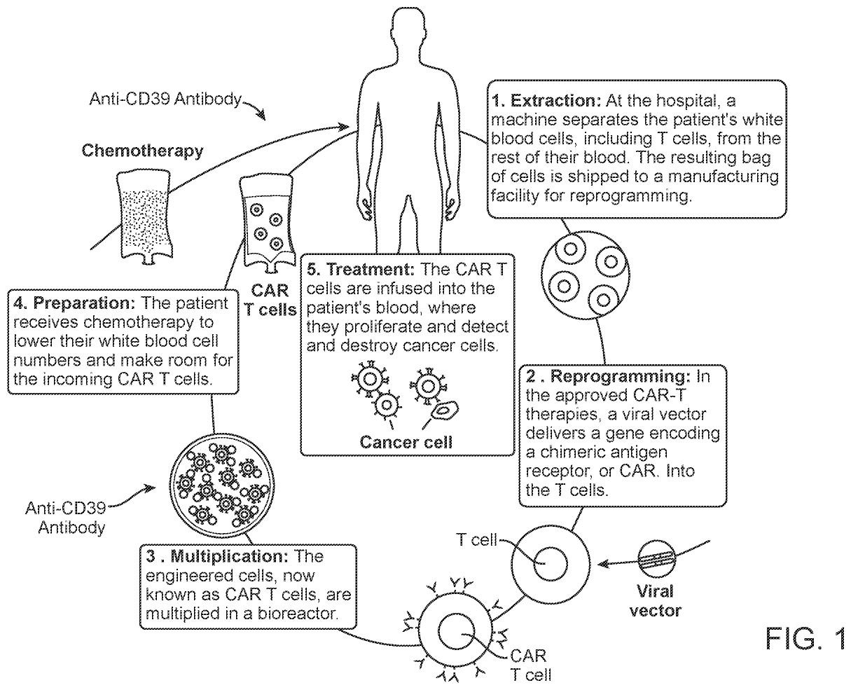

A subject may receive an initial administration of adoptive cell therapy and subsequent administration of adoptive cell therapy (See, for example, FIG. 1). In some embodiments, the subject receives an initial administration of adoptive cell therapy and one or more subsequent administrations. In some embodiments, the one or more administrations are administered less than 15 about days, less than about 14 days, less than about 13 days, less than about 12 days, less than about 11 days, less than about 10 days, less than about 9 days, less than about 8 days, less than about 7 days, less than about 6 days, less than about 5 days, less than about 4 days, less than about 3 days, less than about 2 days, or less than about a day after the previous administration. In some embodiments, more than one administration of adoptive cell therapy is administered to the subject every week. In some embodiments, the subject receives more than one administration of adoptive cell therapies per week followed by a week of no administration of adoptive cell therapy. In some embodiments, no administration of adoptive cell therapy is followed by one or more additional administrations of adoptive cell therapies. In some embodiment, the subject receives more than one cycle of adoptive cell therapy and the time between each cycle is less than about 10, about 9, about 8, about 7, about 6, about 5, about 4, about 3, about 2, or about 1 days.

In some embodiments, a subject may be conditioned prior to receiving an antibody which binds CD39 and/or adoptive cell therapy. As one example, the subject may receive chemotherapy to lower their white blood cell numbers and make room for the CAR-T cells. As another example, a subject may receive chemotherapy to drive tumor cell death and/or promote release of ATP and homing of CD39 cells to a tumor site. In some embodiments, conditioning comprises or consists of administering cyclophosphamide before administration of adoptive cell transfer or before administration of an antibody which binds CD39.

In some embodiments, adoptive cell therapy is administered to a subject and then subsequently blood is drawn. In some embodiments, adoptive cell therapy is administered to a subject and then subsequently an apheresis performed. The process can be carried out multiple times every few weeks. In some embodiments, the subject may undergo leukapheresis, wherein leukocytes are collected, enriched, or depleted ex-vivo to select and/or isolate the cells of interest, e.g., T cells. Populations of Adoptive Cells

In some embodiments, the adoptive cell therapy comprises or consists one or more adoptive cell therapies. In some embodiments, the one or more adoptive cell therapies comprises or consist of one or more of TIL therapy, TCR therapy, and/or CAR therapy. In some embodiments, the immune effector cells comprise or consist of αβ T cells, γδ T cells, regulatory T cells, NK T cells, NK cells, macrophages and or dendritic cells. In some embodiments the adoptive cell therapies comprise or consist of patient derived autologous immune cells and or donor derived allogeneic immune cells (See, for example, Depil, et al., 2020, Cancer Cell Therapy, 19:185-199; Fisher, et al., 2018, Frontiers in Immunology, 9:1409; Wang, et al., 2020, Cancer Letters, 472:175-180; WO2017019848, which is incorporated by reference herein in its entirety). One skilled in the art would be able to envision any combination of adoptive cell therapies.

In some embodiments, the engineered cells comprise one or more engineered cells comprising or consisting of more extracellular binding moieties that bind one or more TAAs and/or or one or more TSAs. In some embodiments, the one or more TAAs and/or TSAs comprise one or more of MART-1, gp100, CEA, CD-19, NY-ESO-1, MAGE-A3, hTERT, EGFR, mesothelin, HPV, EBV, MCC, Mum-1, β-Catenin, CDK4, and/or ERBB2IP (See, for example, Landscape of Tumor Antigens in T Cell Immunotherapy, J Immunol Dec. 1, 2015, 195 (11) 5117-5122; DOI: doi.org/10.4049/jimmunol.1501657, which is incorporated by reference herein in its entirety).

In some embodiments, the TAA is a B cell antigen. In some embodiments, the TAA is a melanoma antigen. In some embodiments, the melanoma antigen is gp100.

### TIL Therapy

In some embodiments, adoptive cell therapy comprises or consists of TIL therapy. In TIL therapy, lymphocytes that have penetrated a tumor are harvested from a tumor biopsy taken from a subject. These lymphocytes can be expanded directly from the isolated tumor. In another method, following excision of the lymphocytes, DNA isolated from the tumor is sequenced to identify mutations found in the cancer indicating neoantigens. Mutated neoantigens are then inserted into autologous dendritic cells, which are co-cultured with the tumor infiltrating lymphocytes. The tumor infiltrating lymphocytes are assayed for neoantigen recognition. Those tumor infiltrating lymphocytes that do recognize the neoantigen are then selected, expanded, and transfused back into the subject.

### TCR Therapy

In some embodiments, adoptive cell therapy comprises or consists of TCR therapy. In some embodiments, the adoptive cell therapy comprises or consists of immune effector cells comprising or consisting of engineered cells expressing a TCR.

TCR therapy involves engineering a subject's or donor's immune cells to express a specific TCR. In TCR therapy, immune cells are harvested from a subject's or donor's blood. The T cells are genetically modified to express a new T cell receptor targeted to a subject's cancer, for example. T cells are expanded in number and infused back into the subject. TCRs can recognize tumor specific proteins on the inside and outside of cells.

### CAR Therapy

In some embodiments, the adoptive cell therapy composition comprises or consists of immune effector cells comprising or consisting of engineered cells expressing a CAR. In some embodiments, the CAR comprises or consists of an extracellular binding moiety, a transmembrane domain, and an intracellular domain. In some embodiments, the CAR comprises or consists of an extracellular binding moiety and an intracellular domain. In some embodiments, the extracellular binding moiety is linked directly or indirectly to the intracellular domain.

In CAR-T therapy, T cells are harvested from a subject's or donor's blood and genetically modified to express a chimeric antigen receptor. Genetically modified T cells are then expanded in number and infused back into the subject. CAR modifications target genetically modified T cells to a subject's cancer and trigger the T cells to attack when they get to the cancer. CARs and methods for engineering and introducing CARs into cells can be found, for example, in WO 200014257; WO 2013126726; WO 2012129514; WO 2014031687; WO 2013166321; WO 2013071154; WO 2013123061; WO 2014055668; WO 2014031687; WO2017112741; WO 2019213184; US 2002131960; US 2013287748; US 20130149337; U.S. Pat. Nos. 6,410,319; 6,451,995; 7,070,995; 7,265,209; 7,354,762; 7,446,179; 7,446,190; 7,446,191; 8,252,592; 8,324,353; 8,339,645; 8,398,282; and 8,479,118; EP2537416; Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75, Al; Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177), each of which is incorporated by reference in its entirety herein.

In some embodiments, CAR-T therapy can modulate T cell activity. In some embodiments, CAR-T therapy can modulate T cell differentiation and/or homeostasis.

In some embodiments, the extracellular binding moiety and or moieties binds an antigen. In some embodiments, the antigen is a TAA or a TSA. In some embodiments, the antigen comprises or consists of a polypeptide. In some embodiments, the antigen comprises or consists of a carbohydrate or other molecule.

In some embodiments, the TAA or TSA comprises or consists of a B cell antigen. In some embodiments, the antigen comprises or consists of CD20, CD19, CD22, ROR1, CD45, CD21, CD5, CD33, Igkappa, Iglambda, CD79a, CD79b or CD30. In some embodiments, the antigen comprises or consists of CD19. In some embodiments, the TAA or TSA comprises one or more TAAs and/or TSAs. In some embodiments, the TAA and/or TSA comprises or consists of MART-1, gp100, CEA, CD-19, NY-ESO-1, MAGE-A3, hTERT, EGFR, mesothelin, HPV, EBV, MCC, Mum-1, β-Catenin, CDK4, and/or ERBB2IP (See, for example, Landscape of Tumor Antigens in T Cell Immunotherapy, J Immunol Dec. 1, 2015, 195 (11) 5117-5122; DOI: doi.org/10.4049/jimmunol.1501657, which is incorporated by reference herein in its entirety).

In some embodiments, the antigen binding moiety is one or more antibody or antibody fragments. In some embodiments, the antigen binding moiety comprises or consists of antibody or antibody fragment comprising or consisting of the sequence set forth in SEQ ID NO: 258. In some embodiments, the antibody or an antibody fragment binds to an antigen. In some embodiments, the antigen is CD19.

In some such embodiments, the antigen binding moiety further comprises or consists of a spacer containing a portion of an Ig molecule. In some embodiments, the portion of the Ig molecule comprises or consists of a human Ig molecule. In some embodiments, the portion of the Ig molecule comprises or consists of an Ig hinge. In some embodiments, the Ig hinge comprises or consists of a IgG4 hinge.

When one or more extracellular binding moieties binds to an antigen, the extracellular binding moiety or moieties delivers an immunostimulatory or immunosupressive signal, such as an ITAM or ITIM-transduced signal, into the cell, thereby promoting or restraining an immune response targeted to a disease or condition. In some embodiments, the transmembrane domain comprises or consists of an ITAM or ITIM.

Transmembrane domains comprise or consist of an a-helix that spans a cell membrane. In some embodiments, the transmembrane domain is naturally associated with one or more domains of a receptor. In some embodiments, the transmembrane domain comprises or consists of a transmembrane domain derived from CD4, CD8a, CD28, and CD3ζ. In some embodiments, the transmembrane domain comprises or consists of alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137 (4-1BB), or CD154.

The CAR includes an intracellular domain. In some embodiments, the intracellular domain comprises or consists of CD3. In some embodiments, the intracellular domain comprises of consists of a human CD3 chain. In some embodiments, the intracellular domain comprises or consists of a CD3ζ signaling domain or functional variant thereof (See, for example, www.uniprot.org/uniprot/P20963.2 and U.S. Pat. Nos. 7,446,190 and 8,911,993, each of which is incorporated by reference herein, including any drawings).

T cell activation is often also mediated by one or more costimulatory domains. In some embodiments, the CAR further comprises or consists of at least one costimulatory domain. In some embodiments, the CAR comprises or consists of a CD28 or 4-1BB costimulatory domain. In some embodiments, the CAR comprises or consists of transmembrane domain, intracellular domain, and/or costimulatory domain of OX40 (CD134), CD27, DAP10, DAP12, and/or ICOS.

In some embodiments, the transmembrane domain and the intracellular domain and/or costimulatory domain are linked. In some embodiments, the intracellular domain and/or the costimulatory domain are linked with an oligo or peptide linker. In some embodiments, the linker is between 2 and 10 amino acids in length. In some embodiments, the linker comprises glycines and/or serine. In some aspects, an additional CAR is expressed in the same engineered cell. In some embodiments, the additional CAR provides secondary or costimulatory signal.

### Cancers

For cancers, the antibodies of the invention are generally administered to a human or a mammal human in a pharmaceutically acceptable dosage form. In some embodiments, the cancer is a hematological cancer. Any suitable cancer may be treated with the antibodies provided herein. In some embodiments, the cancer is a solid cancer. In some embodiments, the cancer is selected from the group consisting of metastatic non-small cell lung cancer (NSCLC), acute lymphoblastic leukemia (ALL), multiple myeloma (MM), acute myeloid leukemia (AML), chronic lymphocytic leukemia (CLL), diffuse large B cell lymphoma (DLBCL), metastatic head and neck squamous cell carcinoma (HNSCC), melanoma, renal cell carcinoma, metastatic cutaneous squamous cell carcinoma, Hodgkin's lymphoma, non-Hodgkin's lymphoma (NHL), nd unresectable or metastatic solid tumor with DNA mismatch repair deficiencies or a microsatellite instability-high state. In some embodiments, the subject is recurrent or progressive after platinum therapy.

### EXAMPLES

### Example 1: CAR Constructs

Mouse FMC63 anti-CD19 scFv (J N Kochenderfer, S A Feldman, Y Zhao, H Xu, M A Black, R A Morgan, W H Wilson, S A Rosenberg. Construction and preclinical evaluation of an anti-CD19 chimeric antigen receptor. J Immunother 32, 689702 (2009), which is incorporated by reference herein in its entirety) was inserted into a second-generation CAR cassette containing a signaling peptide from GM-CSF, a hinge region, transmembrane domain, costimulatory domain from CD28, and the CD3ζ activation domain (CD19 CAR). The FLAG tag (DYKDDDDK) was inserted into the CD19 CAR between the scFv and hinge region (CD19-FLAG CAR).

### Example 2: Generation of CAR-Encoding Lentivirus

DNAs encoding the CARs were synthesized and sub cloned into a third-generation lentiviral vector, Lenti CMV-MCS-EFla-puro by Syno Biological (Beijing, China). All CAR lentiviral constructs were sequenced in both directions to confirm CAR sequence and used for lentivirus production. Ten million growth-arrested HEK293FT cells (Thermo Fisher) were seeded into T75 flasks and cultured overnight, then transfected with the pPACKH1 Lentivector Packaging mix (System Biosciences, Palo Alto, Calif.) and 10 mg of each lentiviral vector using the CalPhos Transfection Kit (Takara, Mountain View, Calif.). The next day the medium was replaced with fresh medium and 48 h later the lentivirus-containing medium was collected. The medium was cleared of cell debris by centrifugation at 2100 g for 30 min. The virus particles were collected by centrifugation at 112,000 g for 100 min, suspended in AIM V-AlbuMAX medium (Thermo Fisher), aliquoted, and frozen at −80° C.

Titers of the virus preparations were determined by quantitative RT-PCR using the Lenti-X qRT-PCR kit (Takara) and the 7900HT thermal cycler (Thermo Fisher). The lentiviral titers were >1×10⁸ pfu/ml. Lentiviruses were generated and used in accordance with approved biosafety level-2 regulations.

### Example 3: Generation and Expansion of CAR-T Cells

PBMC were isolated from human peripheral blood buffy coats (provided by the Stanford University Blood Center in accordance with its approved IRB protocol) suspended at 1×10⁶ cells/ml in AIM V medium containing 10% FBS and 300 U/ml IL-2 (Thermo Fisher), mixed with an equal number (1:1 ratio) of CD3/CD28 Dynabeads (Thermo Fisher), and cultured in non-treated 24-well plates (0.5 ml per well). At 24 and 48 hours, lentivirus was added to the cultures at a multiplicity of infection (MOI) of 5, along with 1 tl of TransPlus transduction enhancer (AlStem). As the T cells proliferated over the next two weeks, the cells were counted every 2-3 days and fresh medium with 300 U/ml IL-2 was added to the cultures to maintain the cell density at 1-3×10⁶ cells/ml.

### Example 4: Xenograft Model for the Evaluation of Anti-CD39 Antibodies in Combination with CAR-T Therapy

Six-week old male NSG mice (Jackson Laboratories, Bar Harbor, Me.) were housed and manipulated in strict accordance with the Institutional Animal Care and Use Committee. On day 0 (n=10 mice/group, 3 groups in total) mice were implanted with 5×10⁵ luciferase expressing Raji cells. On day 2 after implantation, untreated mice received an I.V. bolus of PBS while groups receiving CAR-T cells received 5×10⁶ anti-CD19 CD28-CD3ζ CAR-T cells by tail vein injection.

On day 2 following administration of CAR-T cells, untreated mice were dosed intraperitoneally with PBS. One group of CAR-T treated mice received a 250 μg intraperitoneal dose of huIgG4 isotype control and the test group of CAR-T treated mice received a 250 μg intraperitoneal dose of anti-CD39 antibody (TTX-030). Intra-peritoneal dosing of PBS or 250 μg of antibody was administered every 7 days for the remainder of the study.

Post tumor-implantation tumor progression was quantified by total flux measurements of tumor luminescence using an IVIS Spectrum (Xenogen) imager, following intra-peritoneal injections of D-luciferin substrate. Flow cytometry was performed during the study at indicated time. At the termination of study on day 61, blood, tissue, and bone marrow were collected. Single cell suspensions were prepared from liver, spleen, lymph nodes, and kidney samples by passing through a mesh strainer. Bone marrow was treated with 2 mg/mL collagenase D solution for 30 minutes at 37 degrees Celsius.

Cells were washed with PBS before staining. Blood was transferred to FACs tubes and erythrocytes lysed in fresh ammonium chloride RBC lysis buffer for 10 minutes at room temperature. Cells were pelleted by centrifugation and washed 1 time in FACS buffer (PBS+2% FBS+2 mM EDTA). Samples were blocked using normal mouse serum or Rat Anti-Mouse FcBlock (BD) and human serum for 5 minutes and then stained with anti-CD4 (Biolegend RPA-T4), anti-CD8a (Biolegend RPA-T8), 7AAD (Biolegend), and anti-CD3 (Biolegend OKT3) for 30 minutes, washed two times, and acquired on a FACSCalibur (BD).

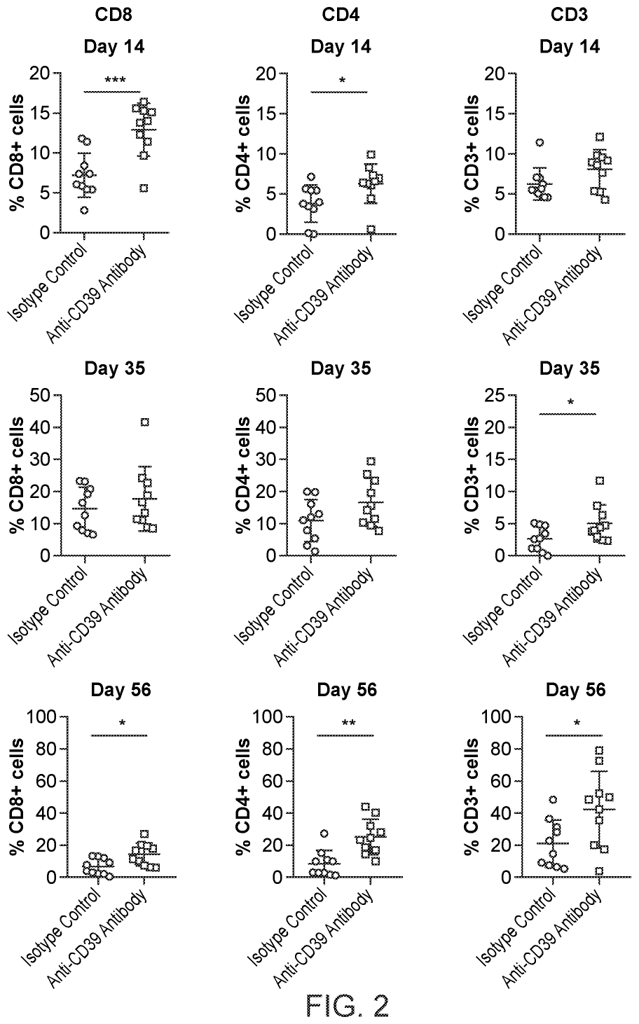

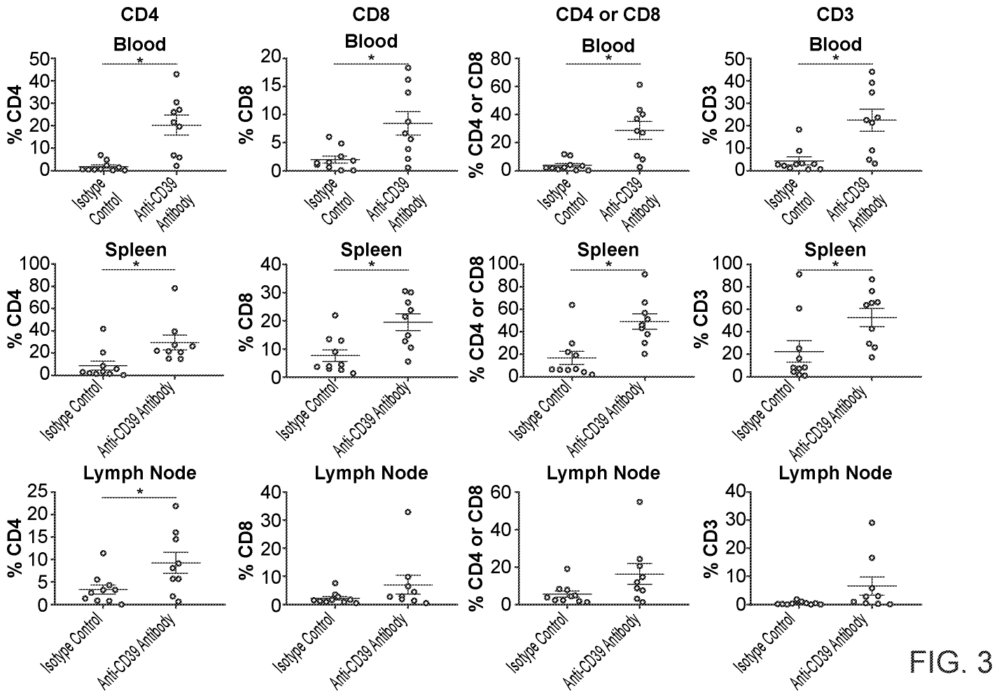

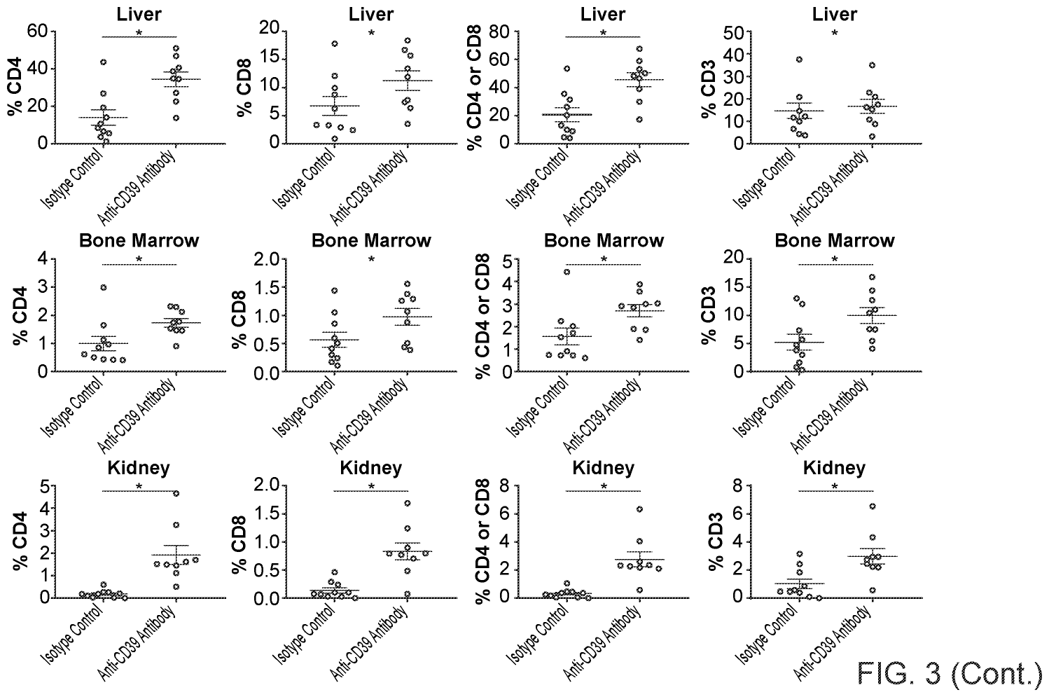

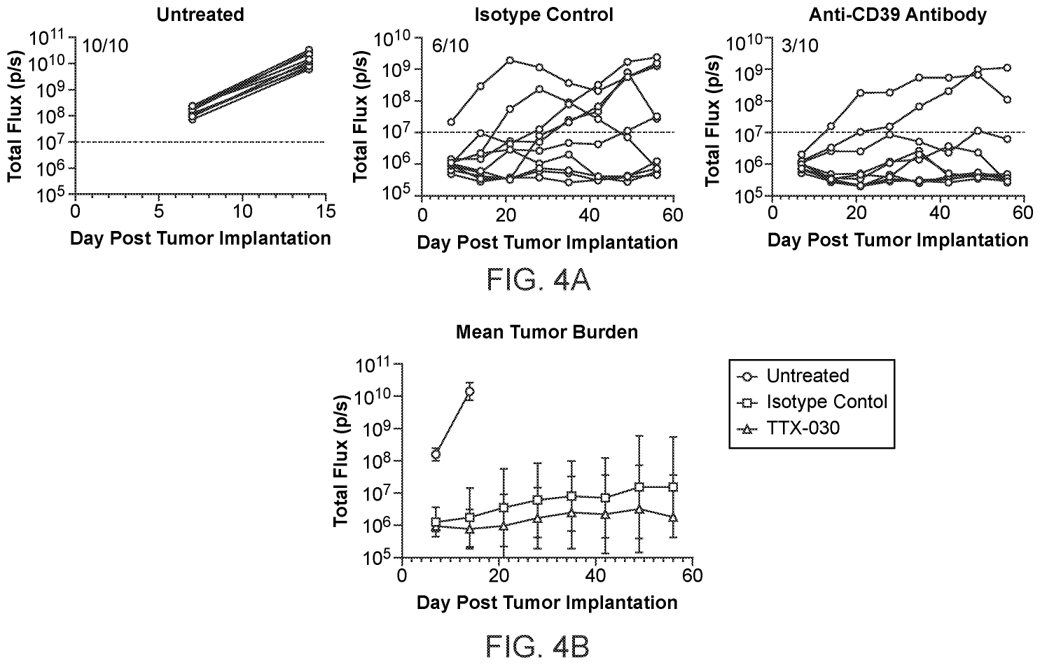

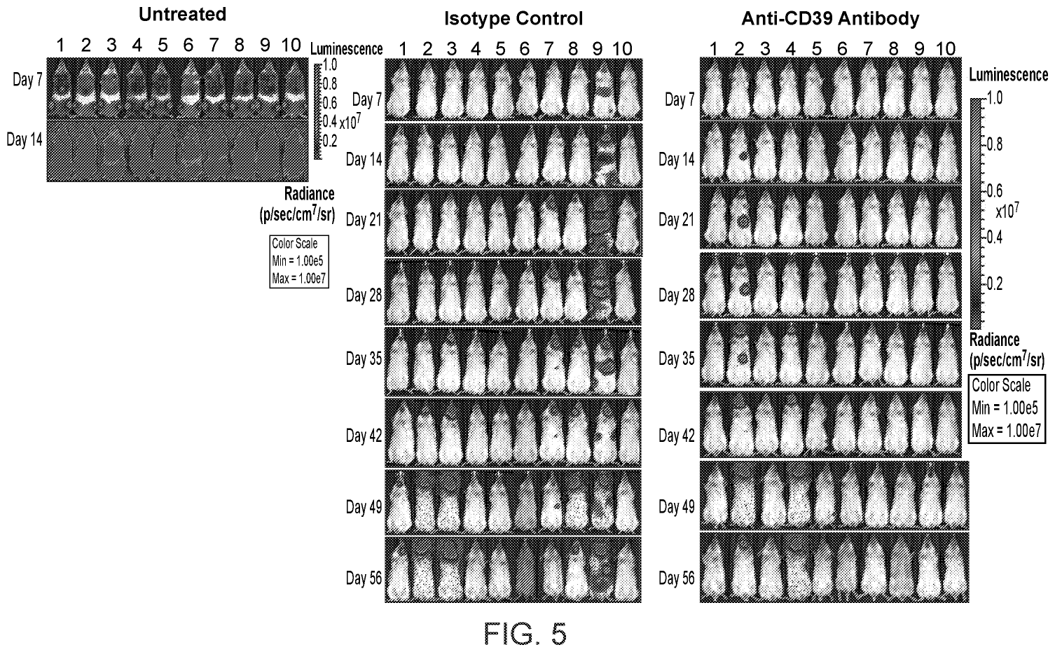

The results can be seen in FIG. 2, FIG. 3, FIG. 4, and FIG. 5. FIG. 2 shows that administration of anti-CD39 antibodies during treatment with CAR-T cells in a mouse xenograft model increases the percentage of CD4+, CD8+, and CD3+ T cells in the blood as measured by flow cytometry at multiple study timepoints capturing CAR-T cell expansion phases and persistence phases. Percentages are shown from individuals with mean percentage indicated ±SEM. Statistics were determined by unpaired t-test (p*<0.05, p**<0.005, p***<0.001). FIG. 3 shows that administration of anti-CD39 antibodies during treatment with CAR-T cells in a mouse xenograft model increases the engraftment of CD4+, CD8+, and CD3+ T cells in peripheral organs at study termination on day 61 as measured by flow cytometry. Percentages are shown from individuals, with mean percentage indicated ±SEM. Statistics were determined by unpaired t-test (p*<0.05, p**<0.005, p***<0.001). FIG. 4 shows decreased tumor burden in a mouse xenograft model using luciferase expressing Raji tumor cells in groups treated with CAR-T in combination with anti-CD39 antibodies as quantified by bioluminescent flux (p/s). (A) shows plotted individual animal flux measurements and (B) shows geometric group mean±SEM flux measurements. Tumor recurrence as determined by flux >5E7 (p/s) are indicated for each treatment population: 10/10 untreated mice, 6/10 CAR-T+isotype control treated mice, and 3/10 CAR-T+anti-CD39 antibodies experienced tumor recurrence). FIG. 5 shows decreased overall tumor burden through IVIS imaging of a mouse xenograft model using luciferase expressing Raji tumor cells in groups treated with CAR-T in combination with anti-CD39 antibodies. Untreated mice were removed from study due to tumor burden following day 14, while mice in both CAR-T treated groups were imaged over the complete study.

### Example 5: Syngeneic B16F10 Model of Tumor Antigen Specific CD8⁺ T Cells Adoptive Cell Transfer in Combination with Anti-CD39 Antibodies

Six week to eight week old female C57BL6 (Charles River Laboratories) mice were housed and manipulated in strict accordance with the Institutional Animal Care and Use Committee. On day −8 (n=15 mice/group, 3 groups in total) mice were implanted intradermally with 1.5×10⁵ B16F10 cells. On day −1 (7 days after implantation) experimental groups received a 250 mg/kg dose of cyclophosphamide as conditioning regimen prior to adoptive transfer. The following day, day 0, the experimental groups received adoptive transfer of 1×10⁶ Pmel-1 CD8+ T cells by tail vein injection.

To generate antigen specific CD8+ T cells for adoptive transfer, splenocytes were isolated from a Pmel-1 mouse (Jackson Laboratories) and stimulated for 16 hours with 1 μM of hgp100₂₅₋₃₃ peptide. Pmel-1 CD8+ T cells were then purified using a MACS CD8a+ T Cell Separation kit (Miltenyi) and washed with PBS. Starting on day 0, untreated mice were dosed intraperitoneally with PBS, while the control group of adoptive cell therapy treated mice received a 250 μg intraperitoneal dose of mIgG1 isotype control and the test group of adoptive cell therapy treated mice received a 250 μg intraperitoneal dose of anti-CD39 antibody (B66).

Intra-peritoneal dosing of PBS or 250 μg of antibody was administered two times per week for the remainder of the study. Post tumor-implantation tumor progression was quantified by caliper measurement. Flow cytometry was performed on a cohort (n=5 mice/group) during the study on day 7 or at the termination of the study on day 35 post adoptive transfer. Mice were humanely euthanized and blood, tissue, and tumor were collected. Single cells were prepared from the spleen, lymph nodes, and tumor samples by passing through a mesh strainer. Cells were washed with PBS before staining. Blood was transferred to FACs tubes and erythrocytes lysed in fresh ammonium chloride RBC lysis buffer for 10 minutes at room temperature. Cells were pelleted by centrifugation and washed two times in FACS buffer (PBS+2% FBS+2 mM EDTA). Samples were blocked with Rat Anti-Mouse FcBlock (BD) and human serum for 20 minutes and then stained with anti-CD4 (Biolegend GK1.5), anti-CD8a (Biolegend 53-6.7), anti-CD45 (Biolegend 30-F11), non-cross blocking anti-CD39 (Biolegend Duha59), anti-Thy1.1 (Biolegend OX-7), anti-Vβ13 (Invitrogen MR12-3), anti-Ki67 (BD B56), and anti-CD3 (BD 17A2) for 30 minutes, washed two times, and acquired on a FACSCalibur (BD).

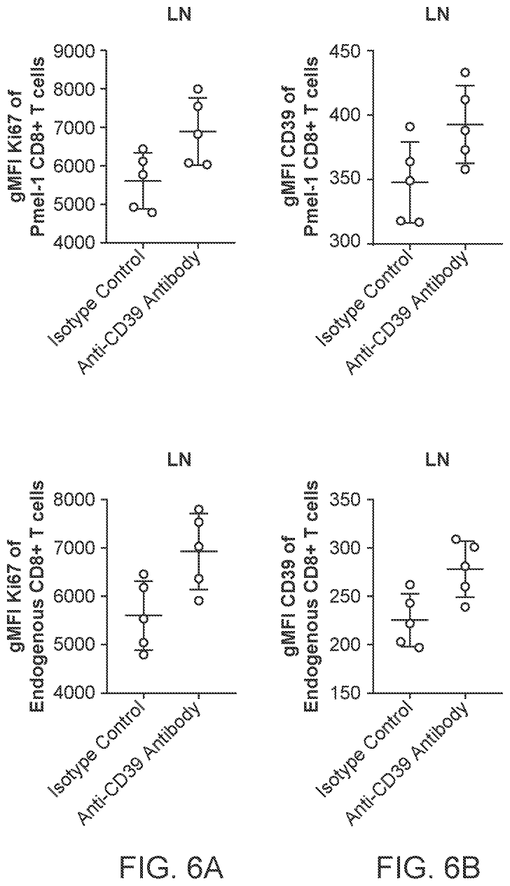

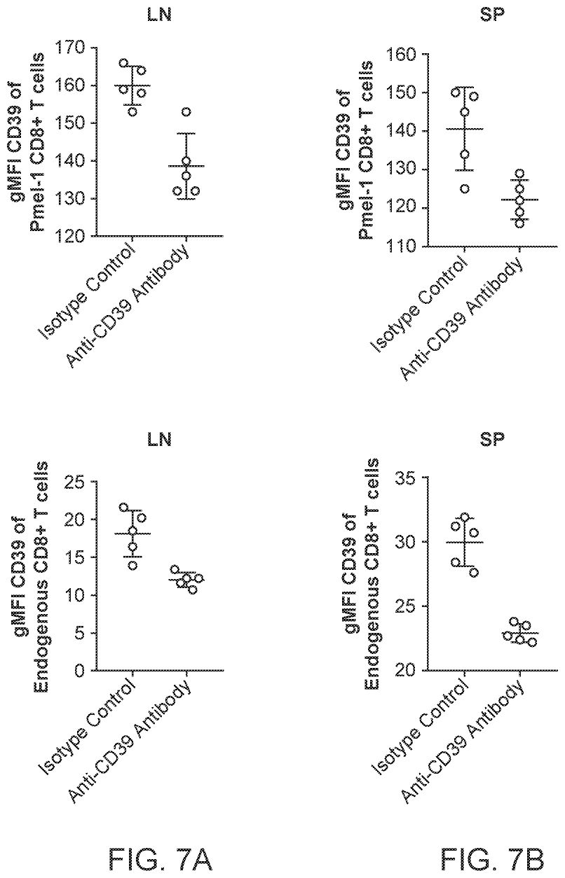

The results can be seen in FIG. 6 and FIG. 7. FIG. 6 (A) Shows that proliferation of adoptively transferred antigen specific CD8+ T cells (top) and endogenous CD8+ T cells (bottom) at the site of antigen presentation in the lymph node is enhanced by treatment with anti-CD39 antibodies as measured by Ki67 staining. FIG. 6 (B) demonstrates improved early activation of adoptively transferred antigen specific CD8+ T cells (top) and endogenous CD8+ T cells (bottom) in the lymph node through treatment with anti-CD39 antibodies as measured by CD39 staining.

### Example 6: B66 Antibody Generation

B66 antibody to murine CD39 was discovered through immunization of WT Sprague Dawley rats followed by screening of hybridomas. Hits were produced as antibodies with Fc region derived from mouse IgG1 and were confirmed for binding to recombinant mCD39 extracellular domain (R&D Systems, cat. #4398-EN) or to mouse cells endogenously expressing CD39. The D265A variant was generated using QuikChange site-directed mutagenesis (Agilent). Anti-human CD39 (TTX-030) was previously generated via yeast display with a human Fab library and expressed by standard techniques (See, for example, WO/2019/027935, which is incorporated by reference herein in its entirety).

Significance was determined using GraphPad Prism 8.0.1 software by unpaired t-test.

### Example 7: Selection of Antibodies which Bind CD39

CD39 antibodies were selected from a synthetic library of human antibodies presented on the surface of yeast cells in IgG format, as generally described, e.g., in WO2009036379; WO2010105256; WO2012009568; and Xu et al., Protein Eng. Des. Sel., 2013, 26:663-670 (each incorporated by reference in its entirety), and more specifically as provided below. The sequences and characteristics of the ABPs isolated from the recombinant library are provided in Table S.

Eight naive human synthetic yeast libraries each of ⁻10E+09 diversity were propagated as described in WO2009036379; WO2010105256; WO2012009568; and Xu et al., Protein Eng. Des. Sel., 2013, 26:663-670; each incorporated by reference in its entirety. For the first two rounds of selection, a magnetic bead sorting technique utilizing the Miltenyi MACS® system was performed, as described in Siegel et al., J. Immunol. Meth., 2004, 286:141-153. The following rounds of selection were performed using flow cytometry based sorting. For all rounds of selection, the antigen was biotinylated human CD39 extracellular domain (heretofore “ECD”), and decreasing concentrations of antigen were used in each subsequent round of selection. In addition to selection on antigen, some rounds of selection were employed in order to reduce the number of non-specific binders utilizing soluble membrane proteins from CHO cells (see WO2014179363 and Xu et al., Protein Eng. Des. Sel., 2013, 26:663-670, each incorporated by reference in its entirety). After the final round of sorting, yeast were plated and individual colonies were picked for characterization and for nomination of clones for affinity maturation.

Antibody variable domains of interest were synthesized, with codon optimization to maximize transient expression in host cells. The variable regions were cloned in to expression vectors containing human immunoglobulin constant domains and their sequence confirmed. Antibody heavy and light chain vector pairings were transfected into Expi293 cells using the Expifectamine system (Invitrogen). Transient cultures were harvested on day 4 and clarified cell culture supernatant IgG titer was estimated using Bio Layer Interferometry (BLI) using Octet (ForteBio) alongside standards. Antibodies were subsequently purified on a Protein A column and eluted using low pH glycine. Purified antibody samples were then buffer-exchanged or dialyzed into downstream assay-compatible buffers.

Antibody purity was assessed by running samples on SDS-PAGE and on an analytical size exclusion chromatography column.

Light Chain Shuffling: Heavy chain plasmids were extracted from naïve outputs (described herein) and transformed into a pre-made naive light chain library with a diversity of 10E+06. Selections were performed as described above with one round of MACS sorting and three rounds of FACS sorting using decreasing amounts of biotinylated ECD antigen for respective rounds. Selected individual heavy chains from the primary discovery process were also independently transformed into separate pre-made light chain libraries with a diversity of 10E+06 and selections performed as described above with one round of MACS sorting and three rounds of FACS sorting using decreasing amount of biotinylated ECD antigen for respective rounds.

### Example 8: Affinity Maturation

Optimization of naive clones was carried out utilizing three maturation strategies; diversification of CDR-H1 and CDR-H2; diversification of CDR-H3; diversification of CDR-L1, L2, and L3; shuffling of diversified heavy and light chains.

CDR-H1 and CDR-H2 Selection: The CDR-H3s from clones selected from each of the light chain batch diversification, light chain diversification, and naive discovery efforts were independently recombined into premade libraries with CDR-H1 and CDR-H2 variants of a diversity of >10E+8 and selections were performed using ECD antigen. Affinity pressures were applied by using decreasing concentrations of antigen.

CDR-H3 Selection: Clones obtained from the CDR-H1 and CDR-H2 selection procedure were subject to additional rounds of affinity maturation via walking dimer mutagenesis of the heavy chain. Selections were performed using ECD as antigen generally as described above but with the addition of employing FACS sorting for all selection rounds.

CDR-L1, L2, L3 Selection: Clones obtained from the CDR-H1 and CDR-H2 selection procedure were subject to additional rounds of affinity maturation via mutagenesis of the light chain. The CDR-L 1 and CDR-L2 diversity derived from a pre-made library while CDR-L3 diversity derived from walking dimer mutagenesis. Selections were performed using ECD as antigen generally as described above but with the addition of employing FACS sorting for all selection rounds, with one round of MACS followed by three rounds of FACS in the CDR-L1, L2, L3 process described here.

Diversified Heavy Chain and Light Chain Shuffling: Outputs from heavy chain diversification and light diversification described above were recombined and selections were performed using ECD as antigen generally as described above but with the addition of employing FACS sorting for all selection rounds.

### Example S: Sequences

Table S provides sequences referred to herein.

TABLE S
Sequences.

| SEQ ID | | | |
|---|---|---|---|
| NO: | Region | Binds | Sequence |
| 1 | CDR-H1 | CD39 | SYYMH |
| 2 | CDR-H1 | CD39 | SYEMH |
| 3 | CDR-H1 | CD39 | SYQMH |
| 4 | CDR-H1 | CD39 | SYYMY |
| 5 | CDR-H1 | CD39 | SYFMH |
| 6 | CDR-H1 | CD39 | SLAIS |
| 7 | CDR-H1 | CD39 | KLAIS |
| 8 | CDR-H1 | CD39 | HTAIS |
| 9 | CDR-H1 | CD39 | SLPIS |
| 10 | CDR-H1 | CD39 | LLAIS |
| 11 | CDR-H1 | CD39 | SNAIS |
| 12 | CDR-H1 | CD39 | AMAIS |
| 13 | CDR-H1 | CD39 | WLAIS |
| 14 | CDR-H1 | CD39 | SYAIS |
| 15 | CDR-H1 | CD39 | SYGIS |
| 16 | CDR-H1 | CD39 | KYGIS |
| 17 | CDR-H1 | CD39 | NYAIS |
| 18 | CDR-H1 | CD39 | SYATS |
| 19 | CDR-H1 | CD39 | SYAIG |
| 20 | CDR-H1 | CD39 | SYSMN |
| 21 | CDR-H1 | CD39 | SYGMN |
| 22 | | | |
| 23 | | | |
| 24 | CDR-H2 | CD39 | VINPSGGSTSYAQKFQG |
| 25 | CDR-H2 | CD39 | RINPSVGSTWYAQKFQG |
| 26 | CDR-H2 | CD39 | RINPSGGSTWYAQKFQG |
| 27 | CDR-H2 | CD39 | KINPSGGSTWYAQKFQG |
| 28 | CDR-H2 | CD39 | VINPLGGGTSYAQKFQG |
| 29 | CDR-H2 | CD39 | SINPRGGSTSYAQKFQG |
| 30 | CDR-H2 | CD39 | GIIPIFGTANYAQKFQG |
| 31 | CDR-H2 | CD39 | GI-GFGTANYAQKFQG |
| 32 | CDR-H2 | CD39 | GILPIGGTANYAQKFQG |
| 33 | CDR-H2 | CD39 | GILPIAGTANYAQKFQG |
| 34 | CDR-H2 | CD39 | GILPIFGEANYAQKFQG |
| 35 | CDR-H2 | CD39 | GIIPRGGTANYAQKFQG |
| 36 | CDR-H2 | CD39 | SIIPIFGTANYAQKFRG |
| 37 | CDR-H2 | CD39 | SIIPEFGIANYAQKFQG |
| 38 | CDR-H2 | CD39 | SIIPIFGTANYAQKFQG |
| 39 | CDR-H2 | CD39 | GIIPISGTANYAQEFQG |
| 40 | CDR-H2 | CD39 | GIIPTFGTANYAQKFQG |
| 41 | CDR-H2 | CD39 | SISSSSSYIYYADSVKG |
| 42 | CDR-H2 | CD39 | VIWYDGSNKYYADSVKG |
| 43 | | | |
| 44 | | | |
| 45 | CDR-H3 | CD39 | GKREGGTEYLRH |
| 46 | CDR-H3 | CD39 | GKREGGTEYLRK |
| 47 | CDR-H3 | CD39 | GKREGGTEYLRS |
| 48 | CDR-H3 | CD39 | GKREGGTEYLRN |
| 49 | CDR-H3 | CD39 | GKREGGTEYLRV |
| 50 | CDR-H3 | CD39 | GGAKYASTYGMDV |
| 51 | CDR-H3 | CD39 | GGAKYASTHGMDV |
| 52 | CDR-H3 | CD39 | GGAKYASQLGMDV |
| 53 | CDR-H3 | CD39 | GGAKYASKWGMDV |
| 54 | CDR-H3 | CD39 | GGAKYAVGYGMDV |
| 55 | CDR-H3 | CD39 | GGAKYAGRYGMDV |
| 56 | CDR-H3 | CD39 | GGAKYARTYGMDV |
| 57 | CDR-H3 | CD39 | ESGGYRDHRLDV |
| 58 | CDR-H3 | CD39 | ESGTYRDHRLDV |
| 59 | CDR-H3 | CD39 | ESGGYRDHRLGV |
| 60 | CDR-H3 | CD39 | DFTDYSSGYSSGWTY |
| 61 | CDR-H3 | CD39 | DTLYSSGAYYGYNV |
| 62 | CDR-H3 | CD39 | AKRGYDSYGGVYFDY |
| 63 | CDR-H3 | CD39 | GPTVTATTSIGTHNWFDP |
| 64 | CDR-H3 | CD39 | EGRGYDSSRYYKFWFDP |
| 65 | CDR-H3 | CD39 | DGGGYRHHYFDL |
| 66 | CDR-H3 | CD39 | ESGGYRDHKLDV |
| 67 | CDR-H3 | CD39 | DGGGYQHHYFDL |
| 68 | CDR-H3 | CD39 | DSGYHRHYSDY |
| 69 | CDR-H3 | CD39 | DPLGIRKHWFDP |
| 70 | CDR-H3 | CD39 | DTPRWRYHYFDY |
| 71 | CDR-H3 | CD39 | ERRGSLALGMDV |
| 72 | CDR-H3 | CD39 | DLGGYSYGEPYYYYYGMDV |
| 73 | | | |
| 74 | | | |
| 75 | CDR-L1 | CD39 | RASQSVSSSYLA |
| 76 | CDR-L1 | CD39 | RASQSVASSYLA |
| 77 | CDR-L1 | CD39 | EASQSVSYSYLA |
| 78 | CDR-L1 | CD39 | KASESVSSSYLA |
| 79 | CDR-L1 | CD39 | RASQYVSSSYLA |
| 80 | CDR-L1 | CD39 | KSSQSVLFSSNNKNYLA |
| 81 | CDR-L1 | CD39 | KSSRSVLFSSNNKNYLA |
| 82 | CDR-L1 | CD39 | KSSKSVLYSNNNKNYLA |
| 83 | CDR-L1 | CD39 | RASQSVGSNLA |
| 84 | CDR-L1 | CD39 | KSSQSVLYSSNNKNYLA |
| 85 | CDR-L1 | CD39 | QASQDISNYLN |
| 86 | CDR-L1 | CD39 | RASQSVSSYLA |
| 87 | CDR-L1 | CD39 | RASQSVSRYLA |
| 88 | CDR-L1 | CD39 | RASQSISSWLA |
| 89 | CDR-L1 | CD39 | RASQSVSSDYLA |
| 90 | | | |
| 91 | | | |
| 92 | CDR-L2 | CD39 | GASSRAT |
| 93 | CDR-L2 | CD39 | GASNRHT |
| 94 | CDR-L2 | CD39 | YASSRAY |
| 95 | CDR-L2 | CD39 | GASSRAN |
| 96 | CDR-L2 | CD39 | YASSRAT |
| 97 | CDR-L2 | CD39 | YASNRAT |
| 98 | CDR-L2 | CD39 | WASTRES |
| 99 | CDR-L2 | CD39 | WASSRES |
| 100 | CDR-L2 | CD39 | WASTRQS |
| 101 | CDR-L2 | CD39 | WASTRAS |
| 102 | CDR-L2 | CD39 | GASTRAT |
| 103 | CDR-L2 | CD39 | GASTRAS |
| 104 | CDR-L2 | CD39 | DASNLET |
| 105 | CDR-L2 | CD39 | DASNRAT |
| 106 | CDR-L2 | CD39 | DASKRAT |
| 107 | CDR-L2 | CD39 | KASSLES |
| 108 | | | |
| 109 | | | |
| 110 | CDR-L3 | CD39 | QQYHSYIT |
| 111 | CDR-L3 | CD39 | QQYHNAIT |
| 112 | CDR-L3 | CD39 | QQYYFYIT |
| 113 | CDR-L3 | CD39 | QQYHSALT |
| 114 | CDR-L3 | CD39 | QQYHGGIT |
| 115 | CDR-L3 | CD39 | QQYHRRIT |
| 116 | CDR-L3 | CD39 | QQYHSGIT |
| 117 | CDR-L3 | CD39 | QQYYLYPLT |
| 118 | CDR-L3 | CD39 | QQYWTYPLT |
| 119 | CDR-L3 | CD39 | QQYLLYPLT |
| 120 | CDR-L3 | CD39 | QQYLIWPLT |
| 121 | CDR-L3 | CD39 | QQYLLWPLT |
| 122 | CDR-L3 | CD39 | QQFYFFPPT |
| 123 | CDR-L3 | CD39 | QQAYTFPPT |
| 124 | CDR-L3 | CD39 | QQYYIFPPT |
| 125 | CDR-L3 | CD39 | QQRNFYPPT |
| 126 | CDR-L3 | CD39 | QQFVLWPRT |
| 127 | CDR-L3 | CD39 | QQHVNFPLT |
| 128 | CDR-L3 | CD39 | QQSVFWPIT |
| 129 | CDR-L3 | CD39 | QQLTKWPLT |
| 130 | CDR-L3 | CD39 | QQDVLWPLT |
| 131 | CDR-L3 | CD39 | QQYGLFPIT |
| 132 | CDR-L3 | CD39 | QQHTVWPIT |
| 133 | CDR-L3 | CD39 | QQVLNYPLT |
| 134 | CDR-L3 | CD39 | QQSYFLPPT |
| 135 | CDR-L3 | CD39 | QQAHSSPYT |
| 136 | | | |
| 137 | | | |
| 138 | VH | CD39 | QVQLVQSGAEVKEPGASVKVSCKAPG |
| | | | YTFTSYYMHWVRQAPGQGLEWMGVIN |
| | | | PSGGSTSYAQKFQGRVTMTRDTSTST |
| | | | VYMELSSLRSEDTAVYYCARGKREGG |
| | | | TEYLRHWGQGTLVTVSS |
| 139 | VH | CD39 | QVQLVQSGAEVKKPGASVKVSCKASG |
| | | | YTFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRKWGQGTLVTVSS |
| 140 | VH | CD39 | QVQLVQSGAEVKKPGASVKVSCKASG |
| | | | YTFTSYQMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRSWGQGTLVTVSS |
| 141 | VH | CD39 | QVQLVQSGAEVKKPGASVKVSCKASG |
| | | | YTFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRNWGQGTLVTVSS |
| 142 | VH | CD39 | QVQLVQSGAEVKKPGASVKVSCKASG |
| | | | YIFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRVWGQGTLVTVSS |
| 143 | VH | CD39 | QVQLVQSGAEVKKPGASVKVSCKASG |
| | | | YTFQSYYMHWVRQAPGQGLEWMGKIN |
| | | | PSGGSTWYAQKFQGRVTMTRDTSTST |
| | | | VYMELSSLRSEDTAVYYCARGKREGG |
| | | | TEYLRHWGQGTLVTVSS |
| 144 | VH | CD39 | QVQLVQSGAEVKKPGASVKVSCKASG |
| | | | YTFKSYEMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRHWGQGTLVTVSS |
| 145 | VH | CD39 | QVQLVQSGAEVKKPGASVKVSCKASG |
| | | | YTFTSYQMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRHWGQGTLVTVSS |
| 146 | VH | CD39 | QVQLVQSGAEVKKPGASVKVSCKASG |
| | | | YTFFSYYMYWVRQAPGQGLEWMGVIN |
| | | | PLGGGTSYAQKFQGRVTMTRDTSTST |
| | | | VYMELSSLRSEDTAVYYCARGKREGG |
| | | | TEYLRHWGQGTLVTVSS |
| 147 | VH | CD39 | QVQLVQSGAEVKKPGASVKVSCKASG |
| | | | YTFVSYFMHWVRQAPGQGLEWMGSIN |
| | | | PRGGSTSYAQKFQGRVTMTRDTSTST |
| | | | VYMELSSLRSEDTAVYYCARGKREGG |
| | | | TEYLRHWGQGTLVTVSS |
| 148 | VH | CD39 | QVQLVQSGAEVKKPGASVKVSCKASG |
| | | | YTFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRHWGQGTLVTVSS |
| 149 | VH | CD39 | QVQLVQSGAEVKKPGSSVKVSCKASG |
| | | | GTFSSLAISWVRQAPGQGLEWMGGII |
| | | | PIFGTANYAQKFQGRVTITADESTNT |
| | | | AYMELSSLRSEDTAVYYCARGGAKYA |
| | | | STYGMDVWGQGTTVTVSS |
| 150 | VH | CD39 | QVQLVQSGAEVKKPGSSVKVSCKASG |
| | | | GTFSKLAISWVRQAPGQGLEWMGGIG |
| | | | FGTANYAQKFQGRVTITADESASTAY |
| | | | MELSSLRSEDTAVYYCARGGAKYAST |
| | | | HGMDVWGQGTTVTVSS |
| 151 | VH | CD39 | QVQLVQSGAEVKKPGSSVKVSCKASG |
| | | | GTFSHTAISWVRQAPGQGLEWMGGIL |
| | | | PIGGTANYAQKFQGRVTITADESTST |
| | | | AYMELSSLRSEDTAVYYCARGGAKYA |
| | | | SQLGMDVWGQGTTVTVSS |
| 152 | VH | CD39 | QVQLVQSGAEVKKPGSSVKVSCKASG |
| | | | GTFSSLPISWVRQAPGQGLEWMGGIG |
| | | | FGTANYAQKFQGRVTITADESTSTAY |
| | | | MELSSLRSEDTAVYYCARGGAKYASK |
| | | | WGMDVWGQGTTVTVSS |
| 153 | VH | CD39 | QVQLVQSGAEVKKPGSSVKVSCKASG |
| | | | GTFSLLAISWVRQAPGQGLEWMGGIL |
| | | | PIAGTANYAQKFQGRVTITADESTST |
| | | | AYMELSSLRSEDTAVYYCARGGAKYA |
| | | | VGYGMDVWGQGTTVTVSS |
| 154 | VH | CD39 | QVQLVQSGAEVKKPGASVKVSCKASG |
| | | | GTFQSLAISWVRQAPGQGLEWMGGIL |
| | | | PIGGTANYAQKFQGRVTITADESTST |
| | | | AYMELSSLRSEDTAVYYCARGGAKYA |
| | | | GRYGMDVWGQGTTVTVSS |
| 155 | VH | CD39 | QVQLVQSGAEVKKPGSSVKVSCKASG |
| | | | GTFPSNAISWVRQAPGQGLEWMGGIG |
| | | | FGTANYAQKFQGRVTITADESTSTAY |
| | | | MELSSLRSEDTAVYYCARGGAKYART |
| | | | YGMDVWGQGTTVTVSS |
| 156 | VH | CD39 | QVQLVQSGAEVKKPGSSVKVSCKASG |
| | | | GTFSSLPISWVRQAPGQGLEWMGGIG |
| | | | FGTANYAQKFQGRVTITADESTSTAY |
| | | | MELSSLRSEDTAVYYCARGGAKYAGR |
| | | | YGMDVWGQGTTVTVSS |
| 157 | VH | CD39 | QVQLVQSGAEVKKPGSSVKVSCKASG |
| | | | GTFSAMAISWVRQAPGQGLEWMGGIL |
| | | | PIAGTANYAQKFQGRVTITADESTST |
| | | | AYMELSSLRSEDTAVYYCARGGAKYA |
| | | | STYGMDVWGQGTTVTVSS |
| 158 | VH | CD39 | QVQLVQSGAEVKKPGSSVKVSCKASG |
| | | | GTFASLAISWVRQAPGQGLEWMGGIL |
| | | | PIFGEANYAQKFQGRVTITADESTST |
| | | | AYMELSSLRSEDTAVYYCARGGAKYA |
| | | | STYGMDVWGQGTTVTVSS |
| 159 | VH | CD39 | QVQLVQSGAEVKKPGSSVKVSCKASG |
| | | | GTFSWLAISWVRQAPGQGLEWMGGII |
| | | | PRGGTANYAQKFQGRVTITADESTST |
| | | | AYMELSSLRSEDTAVYYCARGGAKYA |
| | | | STYGMDVWGQGTTVTVSS |
| 160 | VH | CD39 | QVQLVQSGAEVKKPGSSVKASCKASG |
| | | | GTFSSYAISWVRQAPGQGLEWMGSII |
| | | | PIFGTANYAQKFRGRVTITADESTST |
| | | | TYMELSSLRSEDTAVYYCARESGGYR |
| | | | DHRLDVWGQGTMVTVSS |
| 161 | VH | CD39 | QVQLVQSGAEVKKPGSSVKVSCKASG |
| | | | GTFGSYGISWVRQAPGQGLEWMGSII |
| | | | PEFGIANYAQKFQGRVTITADESTST |
| | | | AYMELSSLRSEDTAVYYCARESGTYR |
| | | | DHRLDVWGQGTMVTVSS |
| 162 | VH | CD39 | QVQLVQSGAEVKKPGSSVKVSCKASG |
| | | | GTFSKYGISWVRQAPGQGLEWMGSII |
| | | | PEFGIANYAQKFQGRVTITADESTST |
| | | | AYMELSSLRSEDTAVYYCARESGGYR |
| | | | DHRLGVWGQGTMVTVSS |
| 163 | VH | CD39 | QVQLVQSGAEVKKPGSSVKVSCKASG |
| | | | GTFESYGISWVRQAPGQGLEWMGSII |
| | | | PEFGIANYAQKFQGRVTITADESTST |
| | | | TYMELSSLRSEDTAVYYCARESGGYR |
| | | | DHRLDVWGQGTMVTVSS |
| 164 | VH | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDFTDYS |
| | | | SGYSSGWTYWGQGTLVTVSS |
| 165 | VH | CD39 | QVQLVQSGAEVKKPGSSVKVSCKASG |
| | | | GTFSNYAISWVRQAPGQGLEWMGGII |
| | | | PIFGTANYAQKFQGRVTITADESTST |
| | | | AYMELSSLRSEDTAVYYCARDTLYSS |
| | | | GAYYGYNVWGQGTMVTVSS |
| 166 | VH | CD39 | QVQLVQSGAEVKKPGSSVKVSCKASG |
| | | | GTFSNYAISWVRQAPGQGLEWMGGII |
| | | | PIFGTANYAQKFQGRVTITADESTST |
| | | | AYMELSSLRSEDTAVYYCARAKRGYD |
| | | | SYGGVYFDYWGQGTLVTVSS |
| 167 | VH | CD39 | QVQLVQSGAEVKKPGSSVKVSCKASG |
| | | | GTFSNYAISWVRQAPGQGLEWMGGII |
| | | | PIFGTANYAQKFQGRVTITADESTST |
| | | | AYMELSSLRSEDTAVYYCARGPTVTA |
| | | | TTSIGTHNWFDPWGQGTLVTVSS |
| 168 | VH | CD39 | QVQLVQSGAEVKKPGSSVKVSCKASG |
| | | | GTFSSYAISWVRQAPGQGLEWMGSII |
| | | | PIFGTANYAQKFQGRVTITADESTST |
| | | | AYMELSSLRSEDTAVYYCAREGRGYD |
| | | | SSRYYKFWFDPWGQGTLVTVSS |
| 169 | VH | CD39 | QVQLVQSGAEVKEPGSSVKVSCKASG |
| | | | GTFSSYATSWVRQAPGQGLEWMGGII |
| | | | PISGTANYAQEFQGRVTITADESTST |
| | | | AYMELSSLRSEDTAVYYCARDGGGYR |
| | | | HHYFDLWGRGTLVTVSS |
| 170 | VH | CD39 | QVQLVQSGAEVKKPGSSVKVPCKASG |
| | | | GTFSSYAISWVRQAPEQGLEWMGSII |
| | | | PIFGTANYAQKFQGRVTITADESTST |
| | | | AYMELSSLRSEDTAVYYCAGESGGYR |
| | | | DHKLDVWGQGTWTVSs |
| 171 | VH | CD39 | QVQLVQSGAEVKKPGSSVKVSCKASG |
| | | | GAFSSYAIGWVRQAPGQGLEWMGGII |
| | | | PTFGTANYAQKFQGRVTITADESTST |
| | | | AYMELSSLRSEDTAVYYCARDGGGYQ |
| | | | HHYFDLWGRGTLVTVSS |
| 172 | VH | CD39 | QVQLVQSGAEVKKPGSSVKVSCKASG |
| | | | GTFSSYAISWVRQAPGQGLEWMGSII |
| | | | PIFGTANYAQKFQGRVTITADESTST |
| | | | AYMELSSLRSEDTAVYYCARESGGYR |
| | | | DHKLDVWGQGTMVTVSS |
| 173 | VH | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDSGYHR |
| | | | HYSDYWGQGTLVTVSS |
| 174 | VH | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDPLGIR |
| | | | KHWFDPWGQGTLVTVSS |
| 175 | VH | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDTPRWR |
| | | | YHYFDYWGQGTLVTVSS |
| 176 | VH | CD39 | EVQLVESGGGLVKPGGSLRLSCAASG |
| | | | FTFSSYSMNWVRQAPGKGLEWVSSIS |
| | | | SSSSYIYYADSVKGRFTISRDNAKNS |
| | | | LYLQMNSLRAEDTAVYYCARERRGSL |
| | | | ALGMDVWGQGTLVTVSS |
| 177 | VH | CD39 | QVQLVESGGGVVQPGRSLRLSCAASG |
| | | | FTFSSYGMNWVRQAPGKGLEWVAVIW |
| | | | YDGSNKYYADSVKGRFTISRDNSKNT |
| | | | LYLQMNSLRAEDTAVYYCARDLGGYS |
| | | | YGEPYYYYYGMDVWGQGTTVTVSS |
| 178 | | | |
| 179 | | | |
| 180 | VL | CD39 | EIVLTQSPGTLSLSPGERATLSCRAS |
| | | | QSVSSSYLAWYQQKPGQAPRLLIYGA |
| | | | SSRATGIPDRFSGSGSGTDFTLTISR |
| | | | LEPEDFAVYYCQQYHSYITFGGGTKV |
| | | | EIK |
| 181 | VL | CD39 | EIVLTQSPGTLSLSPGERATLSCRAS |
| | | | QSVASSYLAWYQQKPGQAPRLLIYGA |
| | | | SNRHTGIPDRFSGSGSGTDFTLTISR |
| | | | LEPEDFAVYYCQQYHNAITFGGGTKV |
| | | | EIK |
| 182 | VL | CD39 | EIVLTQSPGTLSLSPGERATLSCRAS |
| | | | QSVSSSYLAWYQQKPGQAPRLLIYYA |
| | | | SSRAYGIPDRFSGSGSGTDFTLTISR |
| | | | LEPEDFAVYYCQQYHNAITFGGGTKV |
| | | | EIK |
| 183 | VL | CD39 | EIVLTQSPGTLSLSPGERATLSCRAS |
| | | | QSVSSSYLAWYQQKPGQAPRLLIYGA |
| | | | SSRATGIPDRFSGSGSGTDFTLTISR |
| | | | LEPEDFAVYYCQQYYFYITFGGGTKV |
| | | | EIK |
| 184 | VL | CD39 | EIVLTQSPGTLSLSPGERATLSCEAS |
| | | | QSVSYSYLAWYQQKPGQAPRLLIYGA |
| | | | SSRANGIPDRFSGSGSGTDFTLTISR |
| | | | LEPEDFAVYYCQQYHSALTFGGGTKV |
| | | | EIK |
| 185 | VL | CD39 | EIVLTQSPGTLSLSPGERATLSCRAS |
| | | | QSVASSYLAWYQQKPGQAPRLLIYGA |
| | | | SNRHTGIPDRFSGSGSGTDFTLTISR |
| | | | LEPEDFAVYYCQQYHGGITFGGGTKV |
| | | | EIK |
| 186 | VL | CD39 | EIVLTQSPGTLSLSPGERATLSCKAS |
| | | | ESVSSSYLAWYQQKPGQAPRLLIYYA |
| | | | SSRATGIPDRFSGSGSGTDFTLTISR |
| | | | LEPEDFAVYYCQQYHRRITFGGGTKV |
| | | | EIK |
| 187 | VL | CD39 | EIVLTQSPGTLSLSPGERATLSCRAS |
| | | | QYVSSSYLAWYQQKPGQAPRLLIYYA |
| | | | SNRATGIPDRFSGSGSGTDFTLTISR |
| | | | LEPEDFAVYYCQQYHSGITFGGGTKV |
| | | | EIK |
| 188 | VL | CD39 | DIVMTQSPDSLAVSLGERATINCKSS |
| | | | QSVLFSSNNKNYLAWYQQKPGQPPKL |
| | | | LIYWASTRESGVPDRFSGSGSGTDFT |
| | | | LTISSLQAEDVAVYYCQQYYLYPLTF |
| | | | GGGTKVEIK |
| 189 | VL | CD39 | DIVMTQSPDSLAVSLGERATINCKSS |
| | | | RSVLFSSNNKNYLAWYQQKPGQPPKL |
| | | | LIYWASTRESGVPDRFSGSGSGTDFT |
| | | | LTISSLQAEDVAVYYCQQYWTYPLTF |
| | | | GGGTKVEIK |
| 190 | VL | CD39 | DIVMTQSPDSLAVSLGERATINCKSS |
| | | | QSVLFSSNNKNYLAWYQQKPGQPPKL |
| | | | LIYWASSRESGVPDRFSGSGSGTDFT |
| | | | LTISSLQAEDVAVYYCQQYWTYPLTF |
| | | | GGGTKVEIK |
| 191 | VL | CD39 | DIVMTQSPDSLAVSLGERATINCKSS |
| | | | KSVLYSNNNKNYLAWYQQKPGQPPKL |
| | | | LIYWASTRQSGVPDRFSGSGSGTDFT |
| | | | LTISSLQAEDVAVYYCQQYLLYPLTF |
| | | | GGGTKVEIK |
| 192 | VL | CD39 | GIVMTQSPDSLAVSLGERATINCKSS |
| | | | QSVLFSSNNKNYLAWYQQKPGQPPKL |
| | | | LIYWASTRASGVPDRFSGSGSGTDFT |
| | | | LTISSLQAEDVAVYYCQQYYLYPLTF |
| | | | GGGTKVEIK |
| 193 | VL | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQYLIWPLTFGGGTKV |
| | | | EIK |
| 194 | VL | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQYLLWPLTFGGGTKV |
| | | | EIK |
| 195 | VL | CD39 | EIVMTQSPATLSVSPGERATLSCRAS |
| | | | QSVGSNLAWYQQKPGQAPRLLIYGAS |
| | | | TRASGIPARFSGSGSGTEFTLTISSL |
| | | | QSEDFAVYYCQQYLLWPLTFGGGTKV |
| | | | EIK |
| 196 | VL | CD39 | DIVMTQSPDSLAVSLGERATINCKSS |
| | | | QSVLFSSNNKNYLAWYQQKPGQPPKL |
| | | | LIYWASTRESGVPDRFSGSGSGTDFT |
| | | | LTISSLQAEDVAVYYCQQFYFYPPTF |
| | | | GGGTKVEIK |
| 197 | VL | CD39 | DIVMTQSPDSLAVSLGERATINCKSS |
| | | | QSVLYSSNNKNYLAWYQQKPGQPPKL |
| | | | LIYWASTRESGVPDRFSGSGSGTDFT |
| | | | LTISSLQAEDVAVYYCQQAYTFPPTF |
| | | | GGGTKVEIK |
| 198 | VL | CD39 | DIQMTQSPSSLSASVGDRVTITCQAS |
| | | | QDISNYLNWYQQKPGKAPKLLIYDAS |
| | | | NLETGVPSRFSGSGSGTDFTFTISSL |
| | | | QPEDIATYYCQQYYIFPPTFGGGTKV |
| | | | EIK |
| 199 | VL | CD39 | EIVLTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQRNFYPPTFGGGTKV |
| | | | EIK |
| 200 | VL | CD39 | EIVLTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQFVLWPRTFGGGTKV |
| | | | EIK |
| 201 | VL | CD39 | EIVLTQSPATLSLSPGERATLSCRAS |
| | | | QSVSRYLAWYQQKPGQAPRLLIYDAS |
| | | | NRATGIPARFSGSGSGTDFTLTISSL |
| | | | EPEDFAVYYCQQHVNFPLTFGGGTKV |
| | | | EIK |
| 202 | VL | CD39 | EIVLTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQSVFWPITFGGGTKV |
| | | | EIK |
| 203 | VL | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQLTKWPLTFGGGTKV |
| | | | EIK |
| 204 | VL | CD39 | EIVLTQSPATLSLSPGERATLSCRAS |
| | | | QSVSSYLAWYQQKPGQAPRLLIYDAS |
| | | | KRATGIPARFSGSGSGTDFTLTISSL |
| | | | EPEDFAVYYCQQDVLWPLTFGGGTKV |
| | | | EIK |
| 205 | VL | CD39 | DIQMTQSPSTLSASVGDRVTITCRAS |
| | | | QSISSWLAWYQQKPGKAPKLLIYKAS |
| | | | SLESGVPSRFSGSGSGTEFTLTISSL |
| | | | QPDDFATYYCQQYGLFPITFGGGTKV |
| | | | EIK |
| 206 | VL | CD39 | EIVMTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQHTVWPITFGGGTKV |
| | | | EIK |
| 207 | VL | CD39 | EIVLTQSPATLSLSPGERATLSCRAS |
| | | | QSVSSYLAWYQQKPGQAPRLLIYDAS |
| | | | KRATGIPARFSGSGSGTDFTLTISSL |
| | | | EPEDFAVYYCQQVLNYPLTFGGGTKV |
| | | | EIK |
| 208 | VL | CD39 | DIQMTQSPSSLSASVGDRVTITCQAS |
| | | | QDISNYLNWYQQKPGKAPKLLIYDAS |
| | | | NLETGVPSRFSGSGSGTDFTFTISSL |
| | | | QPEDIATYYCQQSYFLPPTFGGGTKV |
| | | | EIK |
| 209 | VL | CD39 | EIVLTQSPGTLSLSPGERATLSCRAS |
| | | | QSVSSDYLAWYQQKPGQAPRLLIYGA |
| | | | SSRATGIPDRFSGSGSGTDFTLTISR |
| | | | LEPEDFAVYYCQQAHSSPYTFGGGTK |
| | | | VEIK |
| 210 | | | |
| 211 | | | |
| 212 | HC | CD39 | QVQLVQSGAEVKKPGASVKVSCKASG |
| | | | YTFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRKWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 213 | LC | CD39 | EIVLTQSPGTLSLSPGERATLSCRAS |
| | | | QSVASSYLAWYQQKPGQAPRLLIYGA |
| | | | SNRHTGIPDRFSGSGSGTDFTLTISR |
| | | | LEPEDFAVYYCQQYHNAITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 214 | HC | CD39 | QVQLVQSGAEVKKPGASVKVSCKASG |
| | | | YTFTSYQMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRSWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 215 | LC | CD39 | EIVLTQSPGTLSLSPGERATLSCRAS |
| | | | QSVSSSYLAWYQQKPGQAPRLLIYGA |
| | | | SSRATGIPDRFSGSGSGTDFTLTISR |
| | | | LEPEDFAVYYCQQYHSYITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 216 | HC | CD39 | QVQLVQSGAEVKKPGASVKVSCKASG |
| | | | YIFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRVWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 217 | LC | CD39 | EIVLTQSPGTLSLSPGERATLSCRAS |
| | | | QSVSSSYLAWYQQKPGQAPRLLIYYA |
| | | | SSRAYGIPDRFSGSGSGTDFTLTISR |
| | | | LEPEDFAVYYCQQYHNAITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 218 | HC | CD39 | QVQLVQSGAEVKKPGASVKVSCKASG |
| | | | YTFKSYEMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRHWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 219 | LC | CD39 | EIVLTQSPGTLSLSPGERATLSCRAS |
| | | | QSVSSSYLAWYQQKPGQAPRLLIYGA |
| | | | SSRATGIPDRFSGSGSGTDFTLTISR |
| | | | LEPEDFAVYYCQQYYFYITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 220 | HC | CD39 | QVQLVQSGAEVKKPGASVKVSCKASG |
| | | | YTFKSYEMHWVRQAPGQGLEWMGRIN |
| | | | PSGGSTWYAQKFQGRVTMTRDTSTST |
| | | | VYMELSSLRSEDTAVYYCARGKREGG |
| | | | TEYLRHWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 221 | LC | CD39 | EIVLTQSPGTLSLSPGERATLSCEAS |
| | | | QSVSYSYLAWYQQKPGQAPRLLIYGA |
| | | | SSRANGIPDRFSGSGSGTDFTLTISR |
| | | | LEPEDFAVYYCQQYHSALTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 222 | HC | CD39 | QVQLVQSGAEVKKPGSSVKVSCKASG |
| | | | GTFSSLPISWVRQAPGQGLEWMGGIG |
| | | | FGTANYAQKFQGRVTITADESTSTAY |
| | | | MELSSLRSEDTAVYYCARGGAKYASK |
| | | | WGMDVWGQGTTVTVSSASTKGPSVFP |
| | | | LAPCSRSTSESTAALGCLVKDYFPEP |
| | | | VTVSWNSGALTSGVHTFPAVLQSSGL |
| | | | YSLSSWTVPSSSLGTKTYTCNVDHK |
| | | | PSNTKVDKRVESKYGPPCPPCPAPEF |
| | | | LGGPSVFLFPPKPKDTLMISRTPEVT |
| | | | CVWDVSQEDPEVQFNWYVDGVEVHN |
| | | | AKTKPREEQFNSTYRWSVLTVLHQD |
| | | | WLNGKEYKCKVSNKGLPSSIEKTISK |
| | | | AKGQPREPQVYTLPPSQEEMTKNQVS |
| | | | LTCLVKGFYPSDIAVEWESNGQPENN |
| | | | YKTTPPVLDSDGSFFLYSRLTVDKSR |
| | | | WQEGNVFSCSVMHEALHNHYTQKSLS |
| | | | LSLGK |
| 223 | LC | CD39 | DIVMTQSPDSLAVSLGERATINCKSS |
| | | | QSVLFSSNNKNYLAWYQQKPGQPPKL |
| | | | LIYWASSRESGVPDRFSGSGSGTDFT |
| | | | LTISSLQAEDVAVYYCQQYWTYPLTF |
| | | | GGGTKVEIKRTVAAPSVFIFPPSDEQ |
| | | | LKSGTASWCLLNNFYPREAKVQWKV |
| | | | DNALQSGNSQESVTEQDSKDSTYSLS |
| | | | STLTLSKADYEKHKVYACEVTHQGLS |
| | | | SPVTKSFNRGEC |
| 224 | HC | CD39 | QVQLVQSGAEVKKPGSSVKVSCKASG |
| | | | GTFPSNAISWVRQAPGQGLEWMGGIG |
| | | | FGTANYAQKFQGRVTITADESTSTAY |
| | | | MELSSLRSEDTAVYYCARGGAKYART |
| | | | YGMDVWGQGTTVTVSSASTKGPSVFP |
| | | | LAPCSRSTSESTAALGCLVKDYFPEP |
| | | | VTVSWNSGALTSGVHTFPAVLQSSGL |
| | | | YSLSSWTVPSSSLGTKTYTCNVDHK |
| | | | PSNTKVDKRVESKYGPPCPPCPAPEF |
| | | | LGGPSVFLFPPKPKDTLMISRTPEVT |
| | | | CVWDVSQEDPEVQFNWYVDGVEVHN |
| | | | AKTKPREEQFNSTYRWSVLTVLHQD |
| | | | WLNGKEYKCKVSNKGLPSSIEKTISK |
| | | | AKGQPREPQVYTLPPSQEEMTKNQVS |
| | | | LTCLVKGFYPSDIAVEWESNGQPENN |
| | | | YKTTPPVLDSDGSFFLYSRLTVDKSR |
| | | | WQEGNVFSCSVMHEALHNHYTQKSLS |
| | | | LSLGK |
| 225 | LC | CD39 | DIVMTQSPDSLAVSLGERATINCKSS |
| | | | KSVLYSNNNKNYLAWYQQKPGQPPKL |
| | | | LIYWASTRQSGVPDRFSGSGSGTDFT |
| | | | LTISSLQAEDVAVYYCQQYLLYPLTF |
| | | | GGGTKVEIKRTVAAPSVFIFPPSDEQ |
| | | | LKSGTASWCLLNNFYPREAKVQWKV |
| | | | DNALQSGNSQESVTEQDSKDSTYSLS |
| | | | STLTLSKADYEKHKVYACEVTHQGLS |
| | | | SPVTKSFNRGEC |
| 226 | HC | CD39 | QVQLVQSGAEVKKPGSSVKVSCKASG |
| | | | GTFSSLPISWVRQAPGQGLEWMGGIG |
| | | | FGTANYAQKFQGRVTITADESTSTAY |
| | | | MELSSLRSEDTAVYYCARGGAKYAGR |
| | | | YGMDVWGQGTTVTVSSASTKGPSVFP |
| | | | LAPCSRSTSESTAALGCLVKDYFPEP |
| | | | VTVSWNSGALTSGVHTFPAVLQSSGL |
| | | | YSLSSWTVPSSSLGTKTYTCNVDHK |
| | | | PSNTKVDKRVESKYGPPCPPCPAPEF |
| | | | LGGPSVFLFPPKPKDTLMISRTPEVT |
| | | | CVWDVSQEDPEVQFNWYVDGVEVHN |
| | | | AKTKPREEQFNSTYRWSVLTVLHQD |
| | | | WLNGKEYKCKVSNKGLPSSIEKTISK |
| | | | AKGQPREPQVYTLPPSQEEMTKNQVS |
| | | | LTCLVKGFYPSDIAVEWESNGQPENN |
| | | | YKTTPPVLDSDGSFFLYSRLTVDKSR |
| | | | WQEGNVFSCSVMHEALHNHYTQKSLS |
| | | | LSLGK |
| 227 | LC | CD39 | GIVMTQSPDSLAVSLGERATINCKSS |
| | | | QSVLFSSNNKNYLAWYQQKPGQPPKL |
| | | | LIYWASTRASGVPDRFSGSGSGTDFT |
| | | | LTISSLQAEDVAVYYCQQYYLYPLTF |
| | | | GGGTKVEIKRTVAAPSVFIFPPSDEQ |
| | | | LKSGTASWCLLNNFYPREAKVQWKV |
| | | | DNALQSGNSQESVTEQDSKDSTYSLS |
| | | | STLTLSKADYEKHKVYACEVTHQGLS |
| | | | SPVTKSFNRGEC |
| 228 | HC | CD39 | QVQLVQSGAEVKKPGSSVKVSCKASG |
| | | | GTFGSYGISWVRQAPGQGLEWMGSII |
| | | | PEFGIANYAQKFQGRVTITADESTST |
| | | | AYMELSSLRSEDTAVYYCARESGTYR |
| | | | DHRLDVWGQGTMVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 229 | LC | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQYLLWPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 230 | HC | CD39 | QVQLVQSGAEVKKPGSSVKVSCKASG |
| | | | GTFSKYGISWVRQAPGQGLEWMGSII |
| | | | PEFGIANYAQKFQGRVTITADESTST |
| | | | AYMELSSLRSEDTAVYYCARESGGYR |
| | | | DHRLGVWGQGTMVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 231 | LC | CD39 | EIVMTQSPATLSVSPGERATLSCRAS |
| | | | QSVGSNLAWYQQKPGQAPRLLIYGAS |
| | | | TRASGIPARFSGSGSGTEFTLTISSL |
| | | | QSEDFAVYYCQQYLLWPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 232 | HC | CD39 | QVQLVQSGAEVKKPGSSVKVSCKASG |
| | | | GTFESYGISWVRQAPGQGLEWMGSII |
| | | | PEFGIANYAQKFQGRVTITADESTST |
| | | | TYMELSSLRSEDTAVYYCARESGGYR |
| | | | DHRLDVWGQGTMVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVWDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 233 | LC | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQYLLWPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 234 | HC | CD39 | QVQLVQSGAEVKKPGSSVKVSCKASG |
| | | | GTFSSYAISWVRQAPGQGLEWMGSII |
| | | | PIFGTANYAQKFQGRVTITADESTST |
| | | | AYMELSSLRSEDTAVYYCAREGRGYD |
| | | | SSRYYKFWFDPWGQGTLVTVSSASTK |
| | | | GPSVFPLAPCSRSTSESTAALGCLVK |
| | | | DYFPEPVTVSWNSGALTSGVHTFPAV |
| | | | LQSSGLYSLSSWTVPSSSLGTKTYT |
| | | | CNVDHKPSNTKVDKRVESKYGPPCPP |
| | | | CPAPEFLGGPSVFLFPPKPKDTLMIS |
| | | | RTPEVTCVVVDVSQEDPEVQFNWYVD |
| | | | GVEVHNAKTKPREEQFNSTYRWSVL |
| | | | TVLHQDWLNGKEYKCKVSNKGLPSSI |
| | | | EKTISKAKGQPREPQVYTLPPSQEEM |
| | | | TKNQVSLTCLVKGFYPSDIAVEWESN |
| | | | GQPENNYKTTPPVLDSDGSFFLYSRL |
| | | | TVDKSRWQEGNVFSCSVMHEALHNHY |
| | | | TQKSLSLSLGK |
| 235 | LC | CD39 | EIVLTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQFVLWPRTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 236 | HC | CD39 | QVQLVQSGAEVKEPGSSVKVSCKASG |
| | | | GTFSSYATSWVRQAPGQGLEWMGGII |
| | | | PISGTANYAQEFQGRVTITADESTST |
| | | | AYMELSSLRSEDTAVYYCARDGGGYR |
| | | | HHYFDLWGRGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVVVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 237 | LC | CD39 | EIVLTQSPATLSLSPGERATLSCRAS |
| | | | QSVSRYLAWYQQKPGQAPRLLIYDAS |
| | | | NRATGIPARFSGSGSGTDFTLTISSL |
| | | | EPEDFAVYYCQQHVNFPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 238 | HC | CD39 | QVQLVQSGAEVKKPGSSVKVPCKASG |
| | | | GTFSSYAISWVRQAPEQGLEWMGSII |
| | | | PIFGTANYAQKFQGRVTITADESTST |
| | | | AYMELSSLRSEDTAVYYCAGESGGYR |
| | | | DHKLDVWGQGTWTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCVVVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 239 | LC | CD39 | EIVLTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQSVFWPITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 240 | HC | CD39 | QVQLVQSGAEVKKPGSSVKVSCKASG |
| | | | GAFSSYAIGWVRQAPGQGLEWMGGII |
| | | | PTFGTANYAQKFQGRVTITADESTST |
| | | | AYMELSSLRSEDTAVYYCARDGGGYQ |
| | | | HHYFDLWGRGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 241 | LC | CD39 | EIVMTQSPATLSVSPGERATLSCRAS |
| | | | QSVGSNLAWYQQKPGQAPRLLIYGAS |
| | | | TRATGIPARFSGSGSGTEFTLTISSL |
| | | | QSEDFAVYYCQQLTKWPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 242 | HC | CD39 | QVQLVQSGAEVKKPGSSVKVSCKASG |
| | | | GTFSSYAISWVRQAPGQGLEWMGSII |
| | | | PIFGTANYAQKFQGRVTITADESTST |
| | | | AYMELSSLRSEDTAVYYCARESGGYR |
| | | | DHKLDVWGQGTMVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 243 | LC | CD39 | EIVLTQSPATLSLSPGERATLSCRAS |
| | | | QSVSSYLAWYQQKPGQAPRLLIYDAS |
| | | | KRATGIPARFSGSGSGTDFTLTISSL |
| | | | EPEDFAVYYCQQDVLWPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 244 | HC | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDSGYHR |
| | | | HYSDYWGQGTLVTVSSASTKGPSVFP |
| | | | LAPCSRSTSESTAALGCLVKDYFPEP |
| | | | VTVSWNSGALTSGVHTFPAVLQSSGL |
| | | | YSLSSWTVPSSSLGTKTYTCNVDHK |
| | | | PSNTKVDKRVESKYGPPCPPCPAPEF |
| | | | LGGPSVFLFPPKPKDTLMISRTPEVT |
| | | | CVWDVSQEDPEVQFNWYVDGVEVHN |
| | | | AKTKPREEQFNSTYRWSVLTVLHQD |
| | | | WLNGKEYKCKVSNKGLPSSIEKTISK |
| | | | AKGQPREPQVYTLPPSQEEMTKNQVS |
| | | | LTCLVKGFYPSDIAVEWESNGQPENN |
| | | | YKTTPPVLDSDGSFFLYSRLTVDKSR |
| | | | WQEGNVFSCSVMHEALHNHYTQKSLS |
| | | | LSLGK |
| 245 | LC | CD39 | DIQMTQSPSTLSASVGDRVTITCRAS |
| | | | QSISSWLAWYQQKPGKAPKLLIYKAS |
| | | | SLESGVPSRFSGSGSGTEFTLTISSL |
| | | | QPDDFATYYCQQYGLFPITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 246 | HC | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDPLGIR |
| | | | KHWFDPWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 247 | LC | CD39 | EIVMTQSPATLSLSPGERATLSCRAS |
| | | | QSVSSYLAWYQQKPGQAPRLLIYDAS |
| | | | NRATGIPARFSGSGSGTDFTLTISSL |
| | | | EPEDFAVYYCQQHTVWPITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 248 | HC | CD39 | QVQLVQSGAEVKKPGSSVKVSCKASG |
| | | | GTFSSYAISWVRQAPGQGLEWMGGII |
| | | | PIFGTANYAQKFQGRVTITADESTST |
| | | | AYMELSSLRSEDTAVYYCARDTPRWR |
| | | | YHYFDYWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 249 | LC | CD39 | EIVLTQSPATLSLSPGERATLSCRAS |
| | | | QSVSSYLAWYQQKPGQAPRLLIYDAS |
| | | | KRATGIPARFSGSGSGTDFTLTISSL |
| | | | EPEDFAVYYCQQVLNYPLTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 250 | HC | CD39 | EVQLVESGGGLVKPGGSLRLSCAASG |
| | | | FTFSSYSMNWVRQAPGKGLEWVSSIS |
| | | | SSSSYIYYADSVKGRFTISRDNAKNS |
| | | | LYLQMNSLRAEDTAVYYCARERRGSL |
| | | | ALGMDVWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 251 | LC | CD39 | DIQMTQSPSSLSASVGDRVTITCQAS |
| | | | QDISNYLNWYQQKPGKAPKLLIYDAS |
| | | | NLETGVPSRFSGSGSGTDFTFTISSL |
| | | | QPEDIATYYCQQSYFLPPTFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 252 | HC | CD39 | QVQLVESGGGWQPGRSLRLSCAASG |
| | | | FTFSSYGMNWVRQAPGKGLEWVAVIW |
| | | | YDGSNKYYADSVKGRFTISRDNSKNT |
| | | | LYLQMNSLRAEDTAVYYCARDLGGYS |
| | | | YGEPYYYYYGMDVWGQGTTVTVSSAS |
| | | | TKGPSVFPLAPCSRSTSESTAALGCL |
| | | | VKDYFPEPVTVSWNSGALTSGVHTFP |
| | | | AVLQSSGLYSLSSVVTVPSSSLGTKT |
| | | | YTCNVDHKPSNTKVDKRVESKYGPPC |
| | | | PPCPAPEFLGGPSVFLFPPKPKDTLM |
| | | | ISRTPEVTCVWDVSQEDPEVQFNWY |
| | | | VDGVEVHNAKTKPREEQFNSTYRVVS |
| | | | VLTVLHQDWLNGKEYKCKVSNKGLPS |
| | | | SIEKTISKAKGQPREPQVYTLPPSQE |
| | | | EMTKNQVSLTCLVKGFYPSDIAVEWE |
| | | | SNGQPENNYKTTPPVLDSDGSFFLYS |
| | | | RLTVDKSRWQEGNVFSCSVMHEALHN |
| | | | HYTQKSLSLSLGK |
| 253 | LC | CD39 | EIVLTQSPGTLSLSPGERATLSCRAS |
| | | | QSVSSDYLAWYQQKPGQAPRLLIYGA |
| | | | SSRATGIPDRFSGSGSGTDFTLTISR |
| | | | LEPEDFAVYYCQQAHSSPYTFGGGTK |
| | | | VEIKRTVAAPSVFIFPPSDEQLKSGT |
| | | | ASWCLLNNFYPREAKVQWKVDNALQ |
| | | | SGNSQESVTEQDSKDSTYSLSSTLTL |
| | | | SKADYEKHKVYACEVTHQGLSSPVTK |
| | | | SFNRGEC |
| 254 | HC | CD39 | QVQLVQSGAEVKKPGASVKVSCKASG |
| | | | YTFKSYEMHWVRQAPGQGLEWMGRIN |
| | | | PSVGSTWYAQKFQGRVTMTRDTSTST |
| | | | VYMELSSLRSEDTAVYYCARGKREGG |
| | | | TEYLRNWGQGTLVTVSSASTKGPSVF |
| | | | PLAPCSRSTSESTAALGCLVKDYFPE |
| | | | PVTVSWNSGALTSGVHTFPAVLQSSG |
| | | | LYSLSSWTVPSSSLGTKTYTCNVDH |
| | | | KPSNTKVDKRVESKYGPPCPPCPAPE |
| | | | FLGGPSVFLFPPKPKDTLMISRTPEV |
| | | | TCWVDVSQEDPEVQFNWYVDGVEVH |
| | | | NAKTKPREEQFNSTYRWSVLTVLHQ |
| | | | DWLNGKEYKCKVSNKGLPSSIEKTIS |
| | | | KAKGQPREPQVYTLPPSQEEMTKNQV |
| | | | SLTCLVKGFYPSDIAVEWESNGQPEN |
| | | | NYKTTPPVLDSDGSFFLYSRLTVDKS |
| | | | RWQEGNVFSCSVMHEALHNHYTQKSL |
| | | | SLSLGK |
| 255 | LC | CD39 | EIVLTQSPGTLSLSPGERATLSCRAS |
| | | | QSVSSSYLAWYQQKPGQAPRLLIYGA |
| | | | SSRATGIPDRFSGSGSGTDFTLTISR |
| | | | LEPEDFAVYYCQQYHSYITFGGGTKV |
| | | | EIKRTVAAPSVFIFPPSDEQLKSGTA |
| | | | SWCLLNNFYPREAKVQWKVDNALQS |
| | | | GNSQESVTEQDSKDSTYSLSSTLTLS |
| | | | KADYEKHKVYACEVTHQGLSSPVTKS |
| | | | FNRGEC |
| 256 | | | |
| 257 | | | |
| 258 | scFv | | DIQMTQTTSSLSASLGDRVTISCRAS |
| | | | QDISKYLNWYQQKPDGTVKLLIYHTS |
| | | | RLHSGVPSRFSGSGSGTDYSLTISNL |
| | | | EQEDIATYFCQQGNTLPYTFGGGTKL |
| | | | EITGGGGSGGGGSGGGGSEVKLQESG |
| | | | PGLVAPSQSLSVTCTVSGVSLPDYGV |
| | | | SWIRQPPRKGLEWLGVIWGSETTYYN |
| | | | SALKSRLTIIKDNSKSQVFLKMNSLQ |
| | | | TDDTAIYYCAKHYYYGGSYAMDYWGQ |
| | | | GTSVTVSS |
| 259 | CDR-H1 | CD39 | SYRMN |
| 260 | CDR-H1 | CD39 | DKAIS |
| 261 | CDR-H1 | CD39 | SEGIS |
| 262 | CDR-H1 | CD39 | TYAIG |
| 263 | CDR-H1 | CD39 | SWYMH |
| 264 | | | |
| 265 | CDR-H2 | CD39 | SISSSSSSIWYADSVKG |
| 266 | CDR-H2 | CD39 | SILPIFGTANYAQKFQG |
| 267 | CDR-H2 | CD39 | SILPIFGTANYAQKFQG |
| 268 | CDR-H2 | CD39 | GIIPAFGTANYAQKFQG |
| 269 | CDR-H2 | CD39 | MINPSGGSTKYAQKFQG |
| 270 | | | |
| 271 | CDR-H3 | CD39 | GPRYDSSGYRWRYGMDV |
| 272 | CDR-H3 | CD39 | EAGYYRYRYFDL |
| 273 | CDR-H3 | CD39 | EAGYYRYRYFDL |
| 274 | CDR-H3 | CD39 | DPVRRSPFDI |
| 275 | CDR-H3 | CD39 | DAPFYTWDHYYGMDV |
| 276 | | | |
| 277 | CDR-L1 | CD39 | RASQSISSYLN |
| 278 | CDR-L1 | CD39 | RASQSVSSNLA |
| 279 | CDR-L1 | CD39 | RASQSVSSNLA |
| 280 | CDR-L1 | CD39 | RASQSVSSYLA |
| 281 | CDR-L1 | CD39 | QASQDISNYLN |
| 282 | | | |
| 283 | CDR-L2 | CD39 | AASSLQS |
| 284 | CDR-L2 | CD39 | GASTRAT |
| 285 | CDR-L2 | CD39 | GASTRAT |
| 286 | CDR-L2 | CD39 | DSSNRAT |
| 287 | CDR-L2 | CD39 | DASNLAT |
| 288 | | | |
| 289 | CDR-L3 | CD39 | QQLYVDPPWT |
| 290 | CDR-L3 | CD39 | QQHALWPLT |
| 291 | CDR-L3 | CD39 | QQHALWPLT |
| 292 | CDR-L3 | CD39 | QQSFLWPRT |
| 293 | CDR-L3 | CD39 | QQLYHLPIT |
| 294 | | | |
| 295 | VH | CD39 | EVQLVESGGGLVKPGGSLRLSCAASGFT |
| | | (SRF360) | FSSYRMNWVRQAPGKGLEWVSSISSSSS |
| | | | SIWYADSVKGRFTISRDNAKNSLYLQMN |
| | | | SLRAEDTAVYYCAKGPRYDSSGYRWRYG |
| | | | MDVWGQGTTVSS |
| 296 | VL | CD39 | DIQMTQSPSSLSASVGDRVTITCRASQS |
| | | (SRF360) | ISSYLNWYQQKPGKAPKLLIYAASSLQS |
| | | | GVPSRFSGSGSGTDFTLTISSLQPEDFA |
| | | | TYYCQQLYVDPPWTFGGGTKVEIK |
| 297 | HC | CD39 | EVQLVESGGGLVKPGGSLRLSCAASGFT |
| | | (SRF360)-G4 | FSSYRMNWVRQAPGKGLEWVSSISSSSS |
| | | | SIWYADSVKGRFTISRDNAKNSLYLQMN |
| | | | SLRAEDTAVYYCAKGPRYDSSGYRWRYG |
| | | | MDVWGQGTTVSSASTKGPSVFPLAPCSR |
| | | | STSESTAALGCLVKDYFPEPVTVSWNSG |
| | | | ALTSGVHTFPAVLQSSGLYSLSSWTVP |
| | | | SSSLGTKTYTCNVDHKPSNTKVDKRVES |
| | | | KYGPPCPPCPAPEFLGGPSVFLFPPKPK |
| | | | DTLMISRTPEVTCVWDVSQEDPEVQFN |
| | | | WYVDGVEVHNAKTKPREEQFNSTYRWS |
| | | | VLTVLHQDWLNGKEYKCKVSNKGLPSSI |
| | | | EKTISKAKGQPREPQVYTLPPSQEEMTK |
| | | | NQVSLTCLVKGFYPSDIAVEWESNGQPE |
| | | | NNYKTTPPVLDSDGSFFLYSRLTVDKSR |
| | | | WQEGNVFSCSVMHEALHNHYTQKSLSLS |
| | | | LGK |
| 298 | LC | CD39 | DIQMTQSPSSLSASVGDRVTITCRASQS |
| | | (SRF360) | ISSYLNWYQQKPGKAPKLLIYAASSLQS |
| | | | GVPSRFSGSGSGTDFTLTISSLQPEDFA |
| | | | TYYCQQLYVDPPWTFGGGTKVEIKRTVA |
| | | | APSVFIFPPSDEQLKSGTASWCLLNNF |
| | | | YPREAKVQWKVDNALQSGNSQESVTEQD |
| | | | SKDSTYSLSSTLTLSKADYEKHKVYACE |
| | | | VTHQGLSSPVTKSFNRGEC |
| 299 | VH | CD39 | QVQLVQSGAEVKKPGSSVKVSCKASGGT |
| | | (SRF365) | FSDKAISWVRQAPGQGLEWMGSILPIFG |
| | | | TANYAQKFQGRVTITADESTSTAYMELS |
| | | | SLRSEDTAVYYCAREAGYYRYRYFDLWG |
| | | | RGTLVTVSS |
| 300 | VL | CD39 | EIVMTQSPATLSVSPGERATLSCRASQS |
| | | (SRF365) | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 301 | HC | CD39 | QVQLVQSGAEVKKPGSSVKVSCKASGGT |
| | | (SRF365) | FSDKAISWVRQAPGQGLEWMGSILPIFG |
| | | | TANYAQKFQGRVTITADESTSTAYMELS |
| | | | SLRSEDTAVYYCAREAGYYRYRYFDLWG |
| | | | RGTLVTVSSASTKGPSVFPLAPCSRSTS |
| | | | ESTAALGCLVKDYFPEPVTVSWNSGALT |
| | | | SGVHTFPAVLQSSGLYSLSSWTVPSSS |
| | | | LGTKTYTCNVDHKPSNTKVDKRVESKYG |
| | | | PPCPPCPAPEFLGGPSVFLFPPKPKDTL |
| | | | MISRTPEVTCVWDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 302 | LC | CD39 | EIVMTQSPATLSVSPGERATLSCRASQS |
| | | (SRF365) | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 303 | VH | CD39 | QVQLVQSGAEVKKPGSSVKVSCKASGGT |
| | | (SRF367) | FSSEGISWVRQAPGQGLEWMGSILPIFG |
| | | | TANYAQKFQGRVTITADESTSTAYMELS |
| | | | SLRSEDTAVYYCAREAGYYRYRYFDLWG |
| | | | KGTLVTVSS |
| 304 | VL | CD39 | EIVMTQSPATLSVSPGERATLSCRASQS |
| | | (SRF367) | VSSNLAWYQQKPGQAPRLLIYGASTRAT |
| | | | GIPARFSGSGSGTEFTLTISSLQSEDFA |
| | | | VYYCQQHALWPLTFGGGTKVEIK |
| 305 | HC | CD39 | QVQLVQSGAEVKKPGSSVKVSCKASGGT |
| | | (SRF367) | FSSEGISWVRQAPGQGLEWMGSILPIFG |
| | | | TANYAQKFQGRVTITADESTSTAYMELS |
| | | | SLRSEDTAVYYCAREAGYYRYRYFDLWG |
| | | | KGTLVTVSSASTKGPSVFPLAPCSRSTS |
| | | | ESTAALGCLVKDYFPEPVTVSWNSGALT |
| | | | SGVHTFPAVLQSSGLYSLSSWTVPSSS |
| | | | LGTKTYTCNVDHKPSNTKVDKRVESKYG |
| | | | PPCPPCPAPEFLGGPSVFLFPPKPKDTL |
| | | | MISRTPEVTCVWDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 306 | LC | CD39 | EIVMTQSPATLSVSPGERATLSCRASQS |
| | | (SRF367) | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 307 | VH | CD39 | QVQLVQSGAEVKKPGSSVKVSCKASGGT |
| | | (SRF370) | FSTYAIGWVRQAPGQGLEWMGGIIPAFG |
| | | | TANYAQKFQGRVTITADESTSTAYMELS |
| | | | SLRSEDTAVYYCARDPVRRSPFDIWGQG |
| | | | TMVTVSS |
| 308 | VL | CD39 | EIVLTQSPATLSLSPGERATLSCRASQS |
| | | (SRF370) | VSSYLAWYQQKPGQAPRLLIYDSSNRAT |
| | | | GIPARFSGSGSGTDFTLTISSLEPEDFA |
| | | | VYYCQQSFLWPRTFGGGTKVEIK |
| 309 | HC | CD39 | QVQLVQSGAEVKKPGSSVKVSCKASGGT |
| | | (SRF370) | FSTYAIGWVRQAPGQGLEWMGGIIPAFG |
| | | | TANYAQKFQGRVTITADESTSTAYMELS |
| | | | SLRSEDTAVYYCARDPVRRSPFDIWGQG |
| | | | TMVTVSSASTKGPSVFPLAPCSRSTSES |
| | | | TAALGCLVKDYFPEPVTVSWNSGALTSG |
| | | | VHTFPAVLQSSGLYSLSSWTVPSSSLG |
| | | | TKTYTCNVDHKPSNTKVDKRVESKYGPP |
| | | | CPPCPAPEFLGGPSVFLFPPKPKDTLMI |
| | | | SRTPEVTCVWDVSQEDPEVQFNWYVDG |
| | | | VEVHNAKTKPREEQFNSTYRWSVLTVL |
| | | | HQDWLNGKEYKCKVSNKGLPSSIEKTIS |
| | | | KAKGQPREPQVYTLPPSQEEMTKNQVSL |
| | | | TCLVKGFYPSDIAVEWESNGQPENNYKT |
| | | | TPPVLDSDGSFFLYSRLTVDKSRWQEGN |
| | | | VFSCSVMHEALHNHYTQKSLSLSLGK |
| 310 | LC | CD39 | EIVLTQSPATLSLSPGERATLSCRASQS |
| | | (SRF370) | VSSYLAWYQQKPGQAPRLLIYDSSNRAT |
| | | | GIPARFSGSGSGTDFTLTISSLEPEDFA |
| | | | VYYCQQSFLWPRTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 311 | VH | CD39 | QVQLVQSGAEVKKPGASVKVSCKASGYT |
| | | (SRF399) | FSSWYMHWVRQAPGQGLEWMGMINPSGG |
| | | | STKYAQKFQGRVTMTRDTSTSTVYMELS |
| | | | SLRSEDTAVYYCARDAPFYTWDHYYGMD |
| | | | VWGQGTTVTVSS |
| 312 | VL | CD39 | DIQMTQSPSSLSASVGDRVTITCQASQD |
| | | (SRF399) | ISNYLNWYQQKPGKAPKLLIYDASNLAT |
| | | | GVPSRFSGSGSGTDFTFTISSLQPEDIA |
| | | | TYYCQQLYHLPITFGGGTKVEIK |
| 313 | HC | CD39 | QVQLVQSGAEVKKPGASVKVSCKASGYT |
| | | (SRF399) | FSSWYMHWVRQAPGQGLEWMGMINPSGG |
| | | | STKYAQKFQGRVTMTRDTSTSTVYMELS |
| | | | SLRSEDTAVYYCARDAPFYTWDHYYGMD |
| | | | VWGQGTTVTVSSASTKGPSVFPLAPCSR |
| | | | STSESTAALGCLVKDYFPEPVTVSWNSG |
| | | | ALTSGVHTFPAVLQSSGLYSLSSWTVP |
| | | | SSSLGTKTYTCNVDHKPSNTKVDKRVES |
| | | | KYGPPCPPCPAPEFLGGPSVFLFPPKPK |
| | | | DTLMISRTPEVTCVWDVSQEDPEVQFN |
| | | | WYVDGVEVHNAKTKPREEQFNSTYRWS |
| | | | VLTVLHQDWLNGKEYKCKVSNKGLPSSI |
| | | | EKTISKAKGQPREPQVYTLPPSQEEMTK |
| | | | NQVSLTCLVKGFYPSDIAVEWESNGQPE |
| | | | NNYKTTPPVLDSDGSFFLYSRLTVDKSR |
| | | | WQEGNVFSCSVMHEALHNHYTQKSLSLS |
| | | | LGK |
| 314 | LC | CD39 | DIQMTQSPSSLSASVGDRVTITCQASQD |
| | | (SRF399) | ISNYLNWYQQKPGKAPKLLIYDASNLAT |
| | | | GVPSRFSGSGSGTDFTFTISSLQPEDIA |
| | | | TYYCQQLYHLPITFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A therapeutic composition comprising an antibody which binds to CD39 and an adoptive cell therapy composition.

2. The therapeutic composition according to claim 1, wherein the adoptive cell therapy composition comprises NK cells, T cells, NKT cells, dendritic cells, macrophages, peripheral blood mononuclear cells (PBMCs), or a combination thereof.

3. The therapeutic composition according to claim 1, wherein the adoptive cell therapy composition comprises engineered cells expressing a heterologous T cell receptor (TCR) and/or a chimeric antigen receptor (CAR).

4. The therapeutic composition according to claim 3, wherein the engineered cells are T cells.

5: The therapeutic composition according to claim 3, wherein the engineered cells express a CAR comprising an extracellular binding moiety that specifically binds a tumor-associated antigen (TAA).

6. The therapeutic composition of claim 5, wherein the TAA is a B cell antigen.

7. The therapeutic composition according to claim 6, wherein the CAR comprises an anti-CD19 binding moiety.

8. The therapeutic composition according to claim 5, wherein the TAA is a melanoma antigen.

9. The therapeutic composition according to claim 8, wherein the melanoma antigen is gp100.

10. The therapeutic composition according to claim 5, wherein the CAR comprises an extracellular binding moiety, a transmembrane domain, and an intracellular domain that triggers the activation and/or proliferation of lymphocytes.

11. The therapeutic composition according to claim 10, wherein the intracellular domain comprises a CD3 signaling domain.

12. The therapeutic composition according to claim 10, wherein the CAR further comprises at least one costimulatory domain.

13. The therapeutic composition according to claim 12, wherein the CAR comprises a CD28 or 4-1BB costimulatory domain.

14. The therapeutic composition according to claim 3, wherein the engineered cells express the heterologous TCR.

15. The therapeutic composition according to claim 14, wherein the heterologous TCR specifically binds a TAA.

16. The therapeutic composition according to claim 15, wherein the TAA is a melanoma antigen.

17. The therapeutic composition according to claim 16, wherein TAA is gp100.

18. The therapeutic composition of claim 17, wherein the heterologous TCR is a pmel-1 TCR.

19. The therapeutic composition according to any of the above claims, wherein the adoptive cell therapy composition comprises CD8+ T cells.

20. The therapeutic composition according to claim 1, wherein the antibody which binds to CD39 comprises a heavy chain variable region (VH) and a light chain variable region (VL), the VH and/or VL together comprising 1, 2, 3, 4, 5, or 6 of:
(a) a VHCDR1 having the sequence set forth in any one of SEQ ID NOs: 1-21 or SEQ ID NOs: 259-263,
(b) a VHCDR2 having the sequence set forth in any one of SEQ ID NOs: 24-42 or SEQ ID NOs: 265-269,
(c) a VHCDR3 having the sequence set forth in any one of SEQ ID NOs: 45-72 or SEQ ID NOs: 271-275,
(d) a VLCDR1 having the sequence set forth in any one of SEQ ID NOs: 75-89 or SEQ ID NOs: 277-281,
(e) a VLCDR2 having the sequence set forth in any one of SEQ ID NOs: 92-107 or SEQ ID NOs: 283-287, and
(f) a VLCDR3 having the sequence set forth in any one of SEQ ID NOs: 110-135 or SEQ ID NOs: 289-293.

21-49. (canceled)


SEQUENCE LISTING (314 sequences)

SEQ ID NO: 1 - protein, 5 aa, Artificial Sequence
  Description of Artificial Sequence: Synthetic peptide
SYYMH

SEQ ID NO: 2 - protein, 5 aa, Artificial Sequence
  Description of Artificial Sequence: Synthetic peptide
SYEMH

SEQ ID NO: 3 - protein, 5 aa, Artificial Sequence
  Description of Artificial Sequence: Synthetic peptide
SYQMH

SEQ ID NO: 4 - protein, 5 aa, Artificial Sequence
  Description of Artificial Sequence: Synthetic peptide
SYYMY

SEQ ID NO: 5 - protein, 5 aa, Artificial Sequence
  Description of Artificial Sequence: Synthetic peptide
SYFMH

SEQ ID NO: 6 - protein, 5 aa, Artificial Sequence
  Description of Artificial Sequence: Synthetic peptide
SLAIS

SEQ ID NO: 7 - protein, 5 aa, Artificial Sequence
  Description of Artificial Sequence: Synthetic peptide
KLAIS

SEQ ID NO: 8 - protein, 5 aa, Artificial Sequence
  Description of Artificial Sequence: Synthetic peptide
HTAIS

SEQ ID NO: 9 - protein, 5 aa, Artificial Sequence
  Description of Artificial Sequence: Synthetic peptide
SLPIS

SEQ ID NO: 10 - protein, 5 aa, Artificial Sequence
  Description of Artificial Sequence: Synthetic peptide
LLAIS

SEQ ID NO: 11 - protein, 5 aa, Artificial Sequence
  Description of Artificial Sequence: Synthetic peptide
SNAIS

SEQ ID NO: 12 - protein, 5 aa, Artificial Sequence
  Description of Artificial Sequence: Synthetic peptide
AMAIS

SEQ ID NO: 13 - protein, 5 aa, Artificial Sequence
  Description of Artificial Sequence: Synthetic peptide
WLAIS

SEQ ID NO: 14 - protein, 5 aa, Artificial Sequence
  Description of Artificial Sequence: Synthetic peptide
SYAIS

SEQ ID NO: 15 - protein, 5 aa, Artificial Sequence
  Description of Artificial Sequence: Synthetic peptide
SYGIS

SEQ ID NO: 16 - protein, 5 aa, Artificial Sequence
  Description of Artificial Sequence: Synthetic peptide
KYGIS

SEQ ID NO: 17 - protein, 5 aa, Artificial Sequence
  Description of Artificial Sequence: Synthetic peptide
NYAIS

SEQ ID NO: 18 - protein, 5 aa, Artificial Sequence
  Description of Artificial Sequence: Synthetic peptide
SYATS

SEQ ID NO: 19 - protein, 5 aa, Artificial Sequence
  Description of Artificial Sequence: Synthetic peptide
SYAIG

SEQ ID NO: 20 - protein, 5 aa, Artificial Sequence
  Description of Artificial Sequence: Synthetic peptide
SYSMN

SEQ ID NO: 21 - protein, 5 aa, Artificial Sequence
  Description of Artificial Sequence: Synthetic peptide
SYGMN

SEQ ID NO: 22-23 - skipped (2 entries)

SEQ ID NO: 24 - protein, 17 aa, Artificial Sequence
  Description of Artificial Sequence: Synthetic peptide
VINPSGGSTSYAQKFQG

SEQ ID NO: 25 - protein, 17 aa, Artificial Sequence
  Description of Artificial Sequence: Synthetic peptide
RINPSVGSTWYAQKFQG

SEQ ID NO: 26 - protein, 17 aa, Artificial Sequence
  Description of Artificial Sequence: Synthetic peptide
RINPSGGSTWYAQKFQG

SEQ ID NO: 27 - protein, 17 aa, Artificial Sequence
  Description of Artificial Sequence: Synthetic peptide
KINPSGGSTWYAQKFQG

SEQ ID NO: 28 - protein, 17 aa, Artificial Sequence
  Description of Artificial Sequence: Synthetic peptide
VINPLGGGTSYAQKFQG

SEQ ID NO: 29 - protein, 17 aa, Artificial Sequence
  Description of Artificial Sequence: Synthetic peptide
SINPRGGSTSYAQKFQG

SEQ ID NO: 30 - protein, 17 aa, Artificial Sequence
  Description of Artificial Sequence: Synthetic peptide
GIIPIFGTANYAQKFQG

SEQ ID NO: 31 - protein, 15 aa, Artificial Sequence
  Description of Artificial Sequence: Synthetic peptide
GIGFGTANYAQKFQG

SEQ ID NO: 32 - protein, 17 aa, Artificial Sequence
  Description of Artificial Sequence: Synthetic peptide
GILPIGGTANYAQKFQG

SEQ ID NO: 33 - protein, 17 aa, Artificial Sequence
  Description of Artificial Sequence: Synthetic peptide
GILPIAGTANYAQKFQG

SEQ ID NO: 34 - protein, 17 aa, Artificial Sequence
  Description of Artificial Sequence: Synthetic peptide
GILPIFGEANYAQKFQG

SEQ ID NO: 35 - protein, 17 aa, Artificial Sequence
  Description of Artificial Sequence: Synthetic peptide
GIIPRGGTANYAQKFQG

SEQ ID NO: 36 - protein, 17 aa, Artificial Sequence
  Description of Artificial Sequence: Synthetic peptide
SIIPIFGTANYAQKFRG

SEQ ID NO: 37 - protein, 17 aa, Artificial Sequence
  Description of Artificial Sequence: Synthetic peptide
SIIPEFGIANYAQKFQG

SEQ ID NO: 38 - protein, 17 aa, Artificial Sequence
  Description of Artificial Sequence: Synthetic peptide
SIIPIFGTANYAQKFQG

SEQ ID NO: 39 - protein, 17 aa, Artificial Sequence
  Description of Artificial Sequence: Synthetic peptide
GIIPISGTANYAQEFQG

SEQ ID NO: 40 - protein, 17 aa, Artificial Sequence
  Description of Artificial Sequence: Synthetic peptide
GIIPTFGTANYAQKFQG

SEQ ID NO: 41 - protein, 17 aa, Artificial Sequence
  Description of Artificial Sequence: Synthetic peptide
SISSSSSYIYYADSVKG

SEQ ID NO: 42 - protein, 17 aa, Artificial Sequence
  Description of Artificial Sequence: Synthetic peptide
VIWYDGSNKYYADSVKG

SEQ ID NO: 43-44 - skipped (2 entries)

SEQ ID NO: 45 - protein, 12 aa, Artificial Sequence
  Description of Artificial Sequence: Synthetic peptide
GKREGGTEYLRH

SEQ ID NO: 46 - protein, 12 aa, Artificial Sequence
  Description of Artificial Sequence: Synthetic peptide
GKREGGTEYLRK

SEQ ID NO: 47 - protein, 12 aa, Artificial Sequence
  Description of Artificial Sequence: Synthetic peptide
GKREGGTEYLRS

SEQ ID NO: 48 - protein, 12 aa, Artificial Sequence
  Description of Artificial Sequence: Synthetic peptide
GKREGGTEYLRN

SEQ ID NO: 49 - protein, 12 aa, Artificial Sequence
  Description of Artificial Sequence: Synthetic peptide
GKREGGTEYLRV

SEQ ID NO: 50 - protein, 13 aa, Artificial Sequence
  Description of Artificial Sequence: Synthetic peptide
GGAKYASTYGMDV

SEQ ID NO: 51 - protein, 13 aa, Artificial Sequence
  Description of Artificial Sequence: Synthetic peptide
GGAKYASTHGMDV

SEQ ID NO: 52 - protein, 13 aa, Artificial Sequence
  Description of Artificial Sequence: Synthetic peptide
GGAKYASQLGMDV

SEQ ID NO: 53 - protein, 13 aa, Artificial Sequence
  Description of Artificial Sequence: Synthetic peptide
GGAKYASKWGMDV

SEQ ID NO: 54 - protein, 13 aa, Artificial Sequence
  Description of Artificial Sequence: Synthetic peptide
GGAKYAVGYGMDV

SEQ ID NO: 55 - protein, 13 aa, Artificial Sequence
  Description of Artificial Sequence: Synthetic peptide
GGAKYAGRYGMDV

SEQ ID NO: 56 - protein, 13 aa, Artificial Sequence
  Description of Artificial Sequence: Synthetic peptide
GGAKYARTYGMDV

SEQ ID NO: 57 - protein, 12 aa, Artificial Sequence
  Description of Artificial Sequence: Synthetic peptide
ESGGYRDHRLDV

SEQ ID NO: 58 - protein, 12 aa, Artificial Sequence
  Description of Artificial Sequence: Synthetic peptide
ESGTYRDHRLDV

SEQ ID NO: 59 - protein, 12 aa, Artificial Sequence
  Description of Artificial Sequence: Synthetic peptide
ESGGYRDHRLGV

SEQ ID NO: 60 - protein, 15 aa, Artificial Sequence
  Description of Artificial Sequence: Synthetic peptide
DFTDYSSGYSSGWTY

SEQ ID NO: 61 - protein, 14 aa, Artificial Sequence
  Description of Artificial Sequence: Synthetic peptide
DTLYSSGAYYGYNV

SEQ ID NO: 62 - protein, 15 aa, Artificial Sequence
  Description of Artificial Sequence: Synthetic peptide
AKRGYDSYGGVYFDY

SEQ ID NO: 63 - protein, 18 aa, Artificial Sequence
  Description of Artificial Sequence: Synthetic peptide
GPTVTATTSIGTHNWFDP

SEQ ID NO: 64 - protein, 17 aa, Artificial Sequence
  Description of Artificial Sequence: Synthetic peptide
EGRGYDSSRYYKFWFDP

SEQ ID NO: 65 - protein, 12 aa, Artificial Sequence
  Description of Artificial Sequence: Synthetic peptide
DGGGYRHHYFDL

SEQ ID NO: 66 - protein, 12 aa, Artificial Sequence
  Description of Artificial Sequence: Synthetic peptide
ESGGYRDHKLDV

SEQ ID NO: 67 - protein, 12 aa, Artificial Sequence
  Description of Artificial Sequence: Synthetic peptide
DGGGYQHHYFDL

SEQ ID NO: 68 - protein, 11 aa, Artificial Sequence
  Description of Artificial Sequence: Synthetic peptide
DSGYHRHYSDY

SEQ ID NO: 69 - protein, 12 aa, Artificial Sequence
  Description of Artificial Sequence: Synthetic peptide
DPLGIRKHWFDP

SEQ ID NO: 70 - protein, 12 aa, Artificial Sequence
  Description of Artificial Sequence: Synthetic peptide
DTPRWRYHYFDY

SEQ ID NO: 71 - protein, 12 aa, Artificial Sequence
  Description of Artificial Sequence: Synthetic peptide
ERRGSLALGMDV

SEQ ID NO: 72 - protein, 19 aa, Artificial Sequence
  Description of Artificial Sequence: Synthetic peptide
DLGGYSYGEPYYYYYGMDV

SEQ ID NO: 73-74 - skipped (2 entries)

SEQ ID NO: 75 - protein, 12 aa, Artificial Sequence
  Description of Artificial Sequence: Synthetic peptide
RASQSVSSSYLA

SEQ ID NO: 76 - protein, 12 aa, Artificial Sequence
  Description of Artificial Sequence: Synthetic peptide
RASQSVASSYLA

SEQ ID NO: 77 - protein, 12 aa, Artificial Sequence
  Description of Artificial Sequence: Synthetic peptide
EASQSVSYSYLA

SEQ ID NO: 78 - protein, 12 aa, Artificial Sequence
  Description of Artificial Sequence: Synthetic peptide
KASESVSSSYLA

SEQ ID NO: 79 - protein, 12 aa, Artificial Sequence
  Description of Artificial Sequence: Synthetic peptide
RASQYVSSSYLA

SEQ ID NO: 80 - protein, 17 aa, Artificial Sequence
  Description of Artificial Sequence: Synthetic peptide
KSSQSVLFSSNNKNYLA

SEQ ID NO: 81 - protein, 17 aa, Artificial Sequence
  Description of Artificial Sequence: Synthetic peptide
KSSRSVLFSSNNKNYLA

SEQ ID NO: 82 - protein, 17 aa, Artificial Sequence
  Description of Artificial Sequence: Synthetic peptide
KSSKSVLYSNNNKNYLA

SEQ ID NO: 83 - protein, 11 aa, Artificial Sequence
  Description of Artificial Sequence: Synthetic peptide
RASQSVGSNLA

SEQ ID NO: 84 - protein, 17 aa, Artificial Sequence
  Description of Artificial Sequence: Synthetic peptide
KSSQSVLYSSNNKNYLA

SEQ ID NO: 85 - protein, 11 aa, Artificial Sequence
  Description of Artificial Sequence: Synthetic peptide
QASQDISNYLN

SEQ ID NO: 86 - protein, 11 aa, Artificial Sequence
  Description of Artificial Sequence: Synthetic peptide
RASQSVSSYLA

SEQ ID NO: 87 - protein, 11 aa, Artificial Sequence
  Description of Artificial Sequence: Synthetic peptide
RASQSVSRYLA

SEQ ID NO: 88 - protein, 11 aa, Artificial Sequence
  Description of Artificial Sequence: Synthetic peptide
RASQSISSWLA

SEQ ID NO: 89 - protein, 12 aa, Artificial Sequence
  Description of Artificial Sequence: Synthetic peptide
RASQSVSSDYLA

SEQ ID NO: 90-91 - skipped (2 entries)

SEQ ID NO: 92 - protein, 7 aa, Artificial Sequence
  Description of Artificial Sequence: Synthetic peptide
GASSRAT

SEQ ID NO: 93 - protein, 7 aa, Artificial Sequence
  Description of Artificial Sequence: Synthetic peptide
GASNRHT

SEQ ID NO: 94 - protein, 7 aa, Artificial Sequence
  Description of Artificial Sequence: Synthetic peptide
YASSRAY

SEQ ID NO: 95 - protein, 7 aa, Artificial Sequence
  Description of Artificial Sequence: Synthetic peptide
GASSRAN

SEQ ID NO: 96 - protein, 7 aa, Artificial Sequence
  Description of Artificial Sequence: Synthetic peptide
YASSRAT

SEQ ID NO: 97 - protein, 7 aa, Artificial Sequence
  Description of Artificial Sequence: Synthetic peptide
YASNRAT

SEQ ID NO: 98 - protein, 7 aa, Artificial Sequence
  Description of Artificial Sequence: Synthetic peptide
WASTRES

SEQ ID NO: 99 - protein, 7 aa, Artificial Sequence
  Description of Artificial Sequence: Synthetic peptide
WASSRES

SEQ ID NO: 100 - protein, 7 aa, Artificial Sequence
  Description of Artificial Sequence: Synthetic peptide
WASTRQS

SEQ ID NO: 101 - protein, 7 aa, Artificial Sequence
  Description of Artificial Sequence: Synthetic peptide
WASTRAS

SEQ ID NO: 102 - protein, 7 aa, Artificial Sequence
  Description of Artificial Sequence: Synthetic peptide
GASTRAT

SEQ ID NO: 103 - protein, 7 aa, Artificial Sequence
  Description of Artificial Sequence: Synthetic peptide
GASTRAS

SEQ ID NO: 104 - protein, 7 aa, Artificial Sequence
  Description of Artificial Sequence: Synthetic peptide
DASNLET

SEQ ID NO: 105 - protein, 7 aa, Artificial Sequence
  Description of Artificial Sequence: Synthetic peptide
DASNRAT

SEQ ID NO: 106 - protein, 7 aa, Artificial Sequence
  Description of Artificial Sequence: Synthetic peptide
DASKRAT

SEQ ID NO: 107 - protein, 7 aa, Artificial Sequence
  Description of Artificial Sequence: Synthetic peptide
KASSLES

SEQ ID NO: 108-109 - skipped (2 entries)

SEQ ID NO: 110 - protein, 8 aa, Artificial Sequence
  Description of Artificial Sequence: Synthetic peptide
QQYHSYIT

SEQ ID NO: 111 - protein, 8 aa, Artificial Sequence
  Description of Artificial Sequence: Synthetic peptide
QQYHNAIT

SEQ ID NO: 112 - protein, 8 aa, Artificial Sequence
  Description of Artificial Sequence: Synthetic peptide
QQYYFYIT

SEQ ID NO: 113 - protein, 8 aa, Artificial Sequence
  Description of Artificial Sequence: Synthetic peptide
QQYHSALT

SEQ ID NO: 114 - protein, 8 aa, Artificial Sequence
  Description of Artificial Sequence: Synthetic peptide
QQYHGGIT

SEQ ID NO: 115 - protein, 8 aa, Artificial Sequence
  Description of Artificial Sequence: Synthetic peptide
QQYHRRIT

SEQ ID NO: 116 - protein, 8 aa, Artificial Sequence
  Description of Artificial Sequence: Synthetic peptide
QQYHSGIT

SEQ ID NO: 117 - protein, 9 aa, Artificial Sequence
  Description of Artificial Sequence: Synthetic peptide
QQYYLYPLT

SEQ ID NO: 118 - protein, 9 aa, Artificial Sequence
  Description of Artificial Sequence: Synthetic peptide
QQYWTYPLT

SEQ ID NO: 119 - protein, 9 aa, Artificial Sequence
  Description of Artificial Sequence: Synthetic peptide
QQYLLYPLT

SEQ ID NO: 120 - protein, 9 aa, Artificial Sequence
  Description of Artificial Sequence: Synthetic peptide
QQYLIWPLT

SEQ ID NO: 121 - protein, 9 aa, Artificial Sequence
  Description of Artificial Sequence: Synthetic peptide
QQYLLWPLT

SEQ ID NO: 122 - protein, 9 aa, Artificial Sequence
  Description of Artificial Sequence: Synthetic peptide
QQFYFFPPT

SEQ ID NO: 123 - protein, 9 aa, Artificial Sequence
  Description of Artificial Sequence: Synthetic peptide
QQAYTFPPT

SEQ ID NO: 124 - protein, 9 aa, Artificial Sequence
  Description of Artificial Sequence: Synthetic peptide
QQYYIFPPT

SEQ ID NO: 125 - protein, 9 aa, Artificial Sequence
  Description of Artificial Sequence: Synthetic peptide
QQRNFYPPT

SEQ ID NO: 126 - protein, 9 aa, Artificial Sequence
  Description of Artificial Sequence: Synthetic peptide
QQFVLWPRT

SEQ ID NO: 127 - protein, 9 aa, Artificial Sequence
  Description of Artificial Sequence: Synthetic peptide
QQHVNFPLT

SEQ ID NO: 128 - protein, 9 aa, Artificial Sequence
  Description of Artificial Sequence: Synthetic peptide
QQSVFWPIT

SEQ ID NO: 129 - protein, 9 aa, Artificial Sequence
  Description of Artificial Sequence: Synthetic peptide
QQLTKWPLT

SEQ ID NO: 130 - protein, 9 aa, Artificial Sequence
  Description of Artificial Sequence: Synthetic peptide
QQDVLWPLT

SEQ ID NO: 131 - protein, 9 aa, Artificial Sequence
  Description of Artificial Sequence: Synthetic peptide
QQYGLFPIT

SEQ ID NO: 132 - protein, 9 aa, Artificial Sequence
  Description of Artificial Sequence: Synthetic peptide
QQHTVWPIT

SEQ ID NO: 133 - protein, 9 aa, Artificial Sequence
  Description of Artificial Sequence: Synthetic peptide
QQVLNYPLT

SEQ ID NO: 134 - protein, 9 aa, Artificial Sequence
  Description of Artificial Sequence: Synthetic peptide
QQSYFLPPT

SEQ ID NO: 135 - protein, 9 aa, Artificial Sequence
  Description of Artificial Sequence: Synthetic peptide
QQAHSSPYT

SEQ ID NO: 136-137 - skipped (2 entries)

SEQ ID NO: 138 - protein, 121 aa, Artificial Sequence
  Description of Artificial Sequence: Synthetic polypeptide
QVQLVQSGAEVKEPGASVKVSCKAPGYTFTSYYMHWVRQAPGQGLEWMGVINPSGGSTSYAQKFQGRVTMTRDTSTSTVYMELSSLRSEDTAVYYCARGKREGGTEYLRHWGQGTLVTVSS

SEQ ID NO: 139 - protein, 121 aa, Artificial Sequence
  Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSS

SEQ ID NO: 140 - protein, 121 aa, Artificial Sequence
  Description of Artificial Sequence: Synthetic polypeptide
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSS

SEQ ID NO: 141 - protein, 121 aa, Artificial Sequence
  Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSS

SEQ ID NO: 142 - protein, 121 aa, Artificial Sequence
  Description of Artificial Sequence: Synthetic polypeptide
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSS

SEQ ID NO: 143 - protein, 121 aa, Artificial Sequence
  Description of Artificial Sequence: Synthetic polypeptide
QVQLVQSGAEVKKPGASVKVSCKASGYTFQSYYMHWVRQAPGQGLEWMGKINPSGGSTWYAQKFQGRVTMTRDTSTSTVYMELSSLRSEDTAVYYCARGKREGGTEYLRHWGQGTLVTVSS

SEQ ID NO: 144 - protein, 121 aa, Artificial Sequence
  Description of Artificial Sequence: Synthetic polypeptide
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 145 - protein, 121 aa, Artificial Sequence
  Description of Artificial Sequence: Synthetic polypeptide
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 146 - protein, 121 aa, Artificial Sequence
  Description of Artificial Sequence: Synthetic polypeptide
QVQLVQSGAEVKKPGASVKVSCKASGYTFFSYYMYWVRQAPGQGLEWMGVINPLGGGTSYAQKFQGRVTMTRDTSTSTVYMELSSLRSEDTAVYYCARGKREGGTEYLRHWGQGTLVTVSS

SEQ ID NO: 147 - protein, 121 aa, Artificial Sequence
  Description of Artificial Sequence: Synthetic polypeptide
QVQLVQSGAEVKKPGASVKVSCKASGYTFVSYFMHWVRQAPGQGLEWMGSINPRGGSTSYAQKFQGRVTMTRDTSTSTVYMELSSLRSEDTAVYYCARGKREGGTEYLRHWGQGTLVTVSS

SEQ ID NO: 148 - protein, 121 aa, Artificial Sequence
  Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRHWGQGTLVTVSS

SEQ ID NO: 149 - protein, 122 aa, Artificial Sequence
  Description of Artificial Sequence: Synthetic polypeptide
QVQLVQSGAEVKKPGSSVKVSCKASGGTFSSLAISWVRQAPGQGLEWMGGIIPIFGTANYAQKFQGRVTITADESTNTAYMELSSLRSEDTAVYYCARGGAKYASTYGMDVWGQGTTVTVSS

SEQ ID NO: 150 - protein, 120 aa, Artificial Sequence
  Description of Artificial Sequence: Synthetic polypeptide
QVQLVQSGAEVKKPGSSVKVSCKASGGTFSKLAISWVRQAPGQGLEWMGGIGFGTANYAQKFQGRVTITADESASTAYMELSSLRSEDTAVYYCARGGAKYASTHGMDVWGQGTTVTVSS

SEQ ID NO: 151 - protein, 122 aa, Artificial Sequence
  Description of Artificial Sequence: Synthetic polypeptide
QVQLVQSGAEVKKPGSSVKVSCKASGGTFSHTAISWVRQAPGQGLEWMGGILPIGGTANYAQKFQGRVTITADESTSTAYMELSSLRSEDTAVYYCARGGAKYASQLGMDVWGQGTTVTVSS

SEQ ID NO: 152 - protein, 120 aa, Artificial Sequence
  Description of Artificial Sequence: Synthetic polypeptide
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSS

SEQ ID NO: 153 - protein, 122 aa, Artificial Sequence
  Description of Artificial Sequence: Synthetic polypeptide
QVQLVQSGAEVKKPGSSVKVSCKASGGTFSLLAISWVRQAPGQGLEWMGGILPIAGTANYAQKFQGRVTITADESTSTAYMELSSLRSEDTAVYYCARGGAKYAVGYGMDVWGQGTTVTVSS

SEQ ID NO: 154 - protein, 122 aa, Artificial Sequence
  Description of Artificial Sequence: Synthetic polypeptide
QVQLVQSGAEVKKPGASVKVSCKASGGTFQSLAISWVRQAPGQGLEWMGGILPIGGTANYAQKFQGRVTITADESTSTAYMELSSLRSEDTAVYYCARGGAKYAGRYGMDVWGQGTTVTVSS

SEQ ID NO: 155 - protein, 120 aa, Artificial Sequence
  Description of Artificial Sequence: Synthetic polypeptide
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSS

SEQ ID NO: 156 - protein, 120 aa, Artificial Sequence
  Description of Artificial Sequence: Synthetic polypeptide
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSS

SEQ ID NO: 157 - protein, 122 aa, Artificial Sequence
  Description of Artificial Sequence: Synthetic polypeptide
QVQLVQSGAEVKKPGSSVKVSCKASGGTFSAMAISWVRQAPGQGLEWMGGILPIAGTANYAQKFQGRVTITADESTSTAYMELSSLRSEDTAVYYCARGGAKYASTYGMDVWGQGTTVTVSS

SEQ ID NO: 158 - protein, 122 aa, Artificial Sequence
  Description of Artificial Sequence: Synthetic polypeptide
QVQLVQSGAEVKKPGSSVKVSCKASGGTFASLAISWVRQAPGQGLEWMGGILPIFGEANYAQKFQGRVTITADESTSTAYMELSSLRSEDTAVYYCARGGAKYASTYGMDVWGQGTTVTVSS

SEQ ID NO: 159 - protein, 122 aa, Artificial Sequence
  Description of Artificial Sequence: Synthetic polypeptide
QVQLVQSGAEVKKPGSSVKVSCKASGGTFSWLAISWVRQAPGQGLEWMGGIIPRGGTANYAQKFQGRVTITADESTSTAYMELSSLRSEDTAVYYCARGGAKYASTYGMDVWGQGTTVTVSS

SEQ ID NO: 160 - protein, 121 aa, Artificial Sequence
  Description of Artificial Sequence: Synthetic polypeptide
QVQLVQSGAEVKKPGSSVKASCKASGGTFSSYAISWVRQAPGQGLEWMGSIIPIFGTANYAQKFRGRVTITADESTSTTYMELSSLRSEDTAVYYCARESGGYRDHRLDVWGQGTMVTVSS

SEQ ID NO: 161 - protein, 121 aa, Artificial Sequence
  Description of Artificial Sequence: Synthetic polypeptide
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSS

SEQ ID NO: 162 - protein, 121 aa, Artificial Sequence
  Description of Artificial Sequence: Synthetic polypeptide
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSS

SEQ ID NO: 163 - protein, 121 aa, Artificial Sequence
  Description of Artificial Sequence: Synthetic polypeptide
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSS

SEQ ID NO: 164 - protein, 124 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDFTDYSSGYSSGWTYWGQGTLVTVSS

SEQ ID NO: 165 - protein, 123 aa, Artificial Sequence
  Description of Artificial Sequence: Synthetic polypeptide
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARDTLYSSGAYYGYNVWGQGTMVTVSS

SEQ ID NO: 166 - protein, 124 aa, Artificial Sequence
  Description of Artificial Sequence: Synthetic polypeptide
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARAKRGYDSYGGVYFDYWGQGTLVTVSS

SEQ ID NO: 167 - protein, 127 aa, Artificial Sequence
  Description of Artificial Sequence: Synthetic polypeptide
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGPTVTATTSIGTHNWFDPWGQGTLVTVSS

SEQ ID NO: 168 - protein, 126 aa, Artificial Sequence
  Description of Artificial Sequence: Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSS

SEQ ID NO: 169 - protein, 121 aa, Artificial Sequence
  Description of Artificial Sequence: Synthetic polypeptide
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSS

SEQ ID NO: 170 - protein, 120 aa, Artificial Sequence
  Description of Artificial Sequence: Synthetic polypeptide
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTWTVSS

SEQ ID NO: 171 - protein, 121 aa, Artificial Sequence
  Description of Artificial Sequence: Synthetic polypeptide
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSS

SEQ ID NO: 172 - protein, 121 aa, Artificial Sequence
  Description of Artificial Sequence: Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSS

SEQ ID NO: 173 - protein, 120 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSS

SEQ ID NO: 174 - protein, 121 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSS

SEQ ID NO: 175 - protein, 121 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSS

SEQ ID NO: 176 - protein, 121 aa, Artificial Sequence
  Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSS

SEQ ID NO: 177 - protein, 128 aa, Artificial Sequence
  Description of Artificial Sequence: Synthetic polypeptide
QVQLVESGGGVVQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSS

SEQ ID NO: 178-179 - skipped (2 entries)

SEQ ID NO: 180 - protein, 107 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIK

SEQ ID NO: 181 - protein, 107 aa, Artificial Sequence
  Description of Artificial Sequence: Synthetic polypeptide
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIK

SEQ ID NO: 182 - protein, 107 aa, Artificial Sequence
  Description of Artificial Sequence: Synthetic polypeptide
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIK

SEQ ID NO: 183 - protein, 107 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIK

SEQ ID NO: 184 - protein, 107 aa, Artificial Sequence
  Description of Artificial Sequence: Synthetic polypeptide
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIK

SEQ ID NO: 185 - protein, 107 aa, Artificial Sequence
  Description of Artificial Sequence: Synthetic polypeptide
EIVLTQSPGTLSLSPGERATLSCRASQSVASSYLAWYQQKPGQAPRLLIYGASNRHTGIPDRFSGSGSGTDFTLTISRLEPEDFAVYYCQQYHGGITFGGGTKVEIK

SEQ ID NO: 186 - protein, 107 aa, Artificial Sequence
  Description of Artificial Sequence: Synthetic polypeptide
EIVLTQSPGTLSLSPGERATLSCKASESVSSSYLAWYQQKPGQAPRLLIYYASSRATGIPDRFSGSGSGTDFTLTISRLEPEDFAVYYCQQYHRRITFGGGTKVEIK

SEQ ID NO: 187 - protein, 107 aa, Artificial Sequence
  Description of Artificial Sequence: Synthetic polypeptide
EIVLTQSPGTLSLSPGERATLSCRASQYVSSSYLAWYQQKPGQAPRLLIYYASNRATGIPDRFSGSGSGTDFTLTISRLEPEDFAVYYCQQYHSGITFGGGTKVEIK

SEQ ID NO: 188 - protein, 113 aa, Artificial Sequence
  Description of Artificial Sequence: Synthetic polypeptide
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYLYPLTFGGGTKVEIK

SEQ ID NO: 189 - protein, 113 aa, Artificial Sequence
  Description of Artificial Sequence: Synthetic polypeptide
DIVMTQSPDSLAVSLGERATINCKSSRSVLFSSNNKNYLAWYQQKPGQPPKLLIYWASTRESGVPDRFSGSGSGTDFTLTISSLQAEDVAVYYCQQYWTYPLTFGGGTKVEIK

SEQ ID NO: 190 - protein, 113 aa, Artificial Sequence
  Description of Artificial Sequence: Synthetic polypeptide
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIK

SEQ ID NO: 191 - protein, 113 aa, Artificial Sequence
  Description of Artificial Sequence: Synthetic polypeptide
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIK

SEQ ID NO: 192 - protein, 113 aa, Artificial Sequence
  Description of Artificial Sequence: Synthetic polypeptide
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIK

SEQ ID NO: 193 - protein, 107 aa, Artificial Sequence
  Description of Artificial Sequence: Synthetic polypeptide
EIVMTQSPATLSVSPGERATLSCRASQSVGSNLAWYQQKPGQAPRLLIYGASTRATGIPARFSGSGSGTEFTLTISSLQSEDFAVYYCQQYLIWPLTFGGGTKVEIK

SEQ ID NO: 194 - protein, 107 aa, Artificial Sequence
  Description of Artificial Sequence: Synthetic polypeptide
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIK

SEQ ID NO: 195 - protein, 107 aa, Artificial Sequence
  Description of Artificial Sequence: Synthetic polypeptide
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIK

SEQ ID NO: 196 - protein, 113 aa, Artificial Sequence
  Description of Artificial Sequence: Synthetic polypeptide
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFYFYPPTFGGGTKVEIK

SEQ ID NO: 197 - protein, 113 aa, Artificial Sequence
  Description of Artificial Sequence: Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQAYTFPPTFGGGTKVEIK

SEQ ID NO: 198 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYYIFPPTFGGGTKVEIK

SEQ ID NO: 199 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRNFYPPTFGGGTKVEIK

SEQ ID NO: 200 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIK

SEQ ID NO: 201 - protein, 107 aa, Artificial Sequence
  Description of Artificial Sequence: Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIK

SEQ ID NO: 202 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIK

SEQ ID NO: 203 - protein, 107 aa, Artificial Sequence
  Description of Artificial Sequence: Synthetic polypeptide
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIK

SEQ ID NO: 204 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIK

SEQ ID NO: 205 - protein, 107 aa, Artificial Sequence
  Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIK

SEQ ID NO: 206 - protein, 107 aa, Artificial Sequence
  Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIK

SEQ ID NO: 207 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIK

SEQ ID NO: 208 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIK

SEQ ID NO: 209 - protein, 108 aa, Artificial Sequence
  Description of Artificial Sequence: Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIK

SEQ ID NO: 210-211 - skipped (2 entries)

SEQ ID NO: 212 - protein, 445 aa, Artificial Sequence
  Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 213 aa, Artificial Sequence
  Description of Artificial Sequence: Synthetic polypeptide
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 445 aa, Artificial Sequence
  Description of Artificial Sequence: Synthetic polypeptide
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 215 - protein, 213 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 445 aa, Artificial Sequence
  Description of Artificial Sequence: Synthetic polypeptide
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 217 - protein, 213 aa, Artificial Sequence
  Description of Artificial Sequence: Synthetic polypeptide
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 445 aa, Artificial Sequence
  Description of Artificial Sequence: Synthetic polypeptide
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - protein, 213 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 445 aa, Artificial Sequence; identical to SEQ ID NO: 218
  Description of Artificial Sequence: Synthetic polypeptide

SEQ ID NO: 221 - protein, 213 aa, Artificial Sequence
  Description of Artificial Sequence: Synthetic polypeptide
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 444 aa, Artificial Sequence
  Description of Artificial Sequence: Synthetic polypeptide
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 223 - protein, 219 aa, Artificial Sequence
  Description of Artificial Sequence: Synthetic polypeptide
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 444 aa, Artificial Sequence
  Description of Artificial Sequence: Synthetic polypeptide
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - protein, 219 aa, Artificial Sequence
  Description of Artificial Sequence: Synthetic polypeptide
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 445 aa, Artificial Sequence
  Description of Artificial Sequence: Synthetic polypeptide
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - protein, 219 aa, Artificial Sequence
  Description of Artificial Sequence: Synthetic polypeptide
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 445 aa, Artificial Sequence
  Description of Artificial Sequence: Synthetic polypeptide
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 229 - protein, 213 aa, Artificial Sequence
  Description of Artificial Sequence: Synthetic polypeptide
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 445 aa, Artificial Sequence
  Description of Artificial Sequence: Synthetic polypeptide
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 231 - protein, 213 aa, Artificial Sequence
  Description of Artificial Sequence: Synthetic polypeptide
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 445 aa, Artificial Sequence
  Description of Artificial Sequence: Synthetic polypeptide
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 213 aa, Artificial Sequence; identical to SEQ ID NO: 229
  Description of Artificial Sequence: Synthetic polypeptide

SEQ ID NO: 234 - protein, 450 aa, Artificial Sequence
  Description of Artificial Sequence: Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - protein, 213 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 445 aa, Artificial Sequence
  Description of Artificial Sequence: Synthetic polypeptide
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 237 - protein, 213 aa, Artificial Sequence
  Description of Artificial Sequence: Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 444 aa, Artificial Sequence
  Description of Artificial Sequence: Synthetic polypeptide
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTWTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - protein, 213 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 445 aa, Artificial Sequence
  Description of Artificial Sequence: Synthetic polypeptide
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 241 - protein, 213 aa, Artificial Sequence
  Description of Artificial Sequence: Synthetic polypeptide
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 445 aa, Artificial Sequence
  Description of Artificial Sequence: Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 243 - protein, 213 aa, Artificial Sequence
  Description of Artificial Sequence: Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 444 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 245 - protein, 213 aa, Artificial Sequence
  Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 445 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 247 - protein, 213 aa, Artificial Sequence
  Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 445 aa, Artificial Sequence
  Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 249 - protein, 213 aa, Artificial Sequence
  Description of Artificial Sequence: Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 445 aa, Artificial Sequence
  Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 251 - protein, 213 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 453 aa, Artificial Sequence
  Description of Artificial Sequence: Synthetic polypeptide
QVQLVESGGGWQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 253 - protein, 214 aa, Artificial Sequence
  Description of Artificial Sequence: Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 445 aa, Artificial Sequence
  Description of Artificial Sequence: Synthetic polypeptide
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 255 - protein, 213 aa, Artificial Sequence; identical to SEQ ID NO: 215
  Description of Artificial Sequence: Synthetic polypeptide

SEQ ID NO: 256-257 - skipped (2 entries)

SEQ ID NO: 258 - protein, 242 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 259 - protein, 5 aa, Artificial Sequence
  Description of Artificial Sequence: Synthetic peptide
SYRMN

SEQ ID NO: 260 - protein, 5 aa, Artificial Sequence
  Description of Artificial Sequence: Synthetic peptide
DKAIS

SEQ ID NO: 261 - protein, 5 aa, Artificial Sequence
  Description of Artificial Sequence: Synthetic peptide
SEGIS

SEQ ID NO: 262 - protein, 5 aa, Artificial Sequence
  Description of Artificial Sequence: Synthetic peptide
TYAIG

SEQ ID NO: 263 - protein, 5 aa, Artificial Sequence
  Description of Artificial Sequence: Synthetic peptide
SWYMH

SEQ ID NO: 264 - skipped

SEQ ID NO: 265 - protein, 17 aa, Artificial Sequence
  Description of Artificial Sequence: Synthetic peptide
SISSSSSSIWYADSVKG

SEQ ID NO: 266 - protein, 17 aa, Artificial Sequence
  Description of Artificial Sequence: Synthetic peptide
SILPIFGTANYAQKFQG

SEQ ID NO: 267 - protein, 17 aa, Artificial Sequence
  Description of Artificial Sequence: Synthetic peptide
SILPIFGTANYAQKFQG

SEQ ID NO: 268 - protein, 17 aa, Artificial Sequence
  Description of Artificial Sequence: Synthetic peptide
GIIPAFGTANYAQKFQG

SEQ ID NO: 269 - protein, 17 aa, Artificial Sequence
  Description of Artificial Sequence: Synthetic peptide
MINPSGGSTKYAQKFQG

SEQ ID NO: 270 - skipped

SEQ ID NO: 271 - protein, 17 aa, Artificial Sequence
  Description of Artificial Sequence: Synthetic peptide
GPRYDSSGYRWRYGMDV

SEQ ID NO: 272 - protein, 12 aa, Artificial Sequence
  Description of Artificial Sequence: Synthetic peptide
EAGYYRYRYFDL

SEQ ID NO: 273 - protein, 12 aa, Artificial Sequence
  Description of Artificial Sequence: Synthetic peptide
EAGYYRYRYFDL

SEQ ID NO: 274 - protein, 10 aa, Artificial Sequence
  Description of Artificial Sequence: Synthetic peptide
DPVRRSPFDI

SEQ ID NO: 275 - protein, 15 aa, Artificial Sequence
  Description of Artificial Sequence: Synthetic peptide
DAPFYTWDHYYGMDV

SEQ ID NO: 276 - skipped

SEQ ID NO: 277 - protein, 11 aa, Artificial Sequence
  Description of Artificial Sequence: Synthetic peptide
RASQSISSYLN

SEQ ID NO: 278 - protein, 11 aa, Artificial Sequence
  Description of Artificial Sequence: Synthetic peptide
RASQSVSSNLA

SEQ ID NO: 279 - protein, 11 aa, Artificial Sequence
  Description of Artificial Sequence: Synthetic peptide
RASQSVSSNLA

SEQ ID NO: 280 - protein, 11 aa, Artificial Sequence
  Description of Artificial Sequence: Synthetic peptide
RASQSVSSYLA

SEQ ID NO: 281 - protein, 11 aa, Artificial Sequence
  Description of Artificial Sequence: Synthetic peptide
QASQDISNYLN

SEQ ID NO: 282 - skipped

SEQ ID NO: 283 - protein, 7 aa, Artificial Sequence
  Description of Artificial Sequence: Synthetic peptide
AASSLQS

SEQ ID NO: 284 - protein, 7 aa, Artificial Sequence
  Description of Artificial Sequence: Synthetic peptide
GASTRAT

SEQ ID NO: 285 - protein, 7 aa, Artificial Sequence
  Description of Artificial Sequence: Synthetic peptide
GASTRAT

SEQ ID NO: 286 - protein, 7 aa, Artificial Sequence
  Description of Artificial Sequence: Synthetic peptide
DSSNRAT

SEQ ID NO: 287 - protein, 7 aa, Artificial Sequence
  Description of Artificial Sequence: Synthetic peptide
DASNLAT

SEQ ID NO: 288 - skipped

SEQ ID NO: 289 - protein, 10 aa, Artificial Sequence
  Description of Artificial Sequence: Synthetic peptide
QQLYVDPPWT

SEQ ID NO: 290 - protein, 9 aa, Artificial Sequence
  Description of Artificial Sequence: Synthetic peptide
QQHALWPLT

SEQ ID NO: 291 - protein, 9 aa, Artificial Sequence
  Description of Artificial Sequence: Synthetic peptide
QQHALWPLT

SEQ ID NO: 292 - protein, 9 aa, Artificial Sequence
  Description of Artificial Sequence: Synthetic peptide
QQSFLWPRT

SEQ ID NO: 293 - protein, 9 aa, Artificial Sequence
  Description of Artificial Sequence: Synthetic peptide
QQLYHLPIT

SEQ ID NO: 294 - skipped

SEQ ID NO: 295 - protein, 124 aa, Artificial Sequence
  Description of Artificial Sequence: Synthetic polypeptide
EVQLVESGGGLVKPGGSLRLSCAASGFTFSSYRMNWVRQAPGKGLEWVSSISSSSSSIWYADSVKGRFTISRDNAKNSLYLQMNSLRAEDTAVYYCAKGPRYDSSGYRWRYGMDVWGQGTTVSS

SEQ ID NO: 296 - protein, 108 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQLYVDPPWTFGGGTKVEIK

SEQ ID NO: 297 - protein, 448 aa, Artificial Sequence
  Description of Artificial Sequence: Synthetic polypeptide
EVQLVESGGGLVKPGGSLRLSCAASGFTFSSYRMNWVRQAPGKGLEWVSSISSSSSSIWYADSVKGRFTISRDNAKNSLYLQMNSLRAEDTAVYYCAKGPRYDSSGYRWRYGMDVWGQGTTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 298 - protein, 214 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQLYVDPPWTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 121 aa, Artificial Sequence
  Description of Artificial Sequence: Synthetic polypeptide
QVQLVQSGAEVKKPGSSVKVSCKASGGTFSDKAISWVRQAPGQGLEWMGSILPIFGTANYAQKFQGRVTITADESTSTAYMELSSLRSEDTAVYYCAREAGYYRYRYFDLWGRGTLVTVSS

SEQ ID NO: 300 - protein, 213 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 445 aa, Artificial Sequence
  Description of Artificial Sequence: Synthetic polypeptide
QVQLVQSGAEVKKPGSSVKVSCKASGGTFSDKAISWVRQAPGQGLEWMGSILPIFGTANYAQKFQGRVTITADESTSTAYMELSSLRSEDTAVYYCAREAGYYRYRYFDLWGRGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 302 - protein, 213 aa, Artificial Sequence; identical to SEQ ID NO: 300
  Description of Artificial Sequence: Synthetic polypeptide

SEQ ID NO: 303 - protein, 121 aa, Artificial Sequence
  Description of Artificial Sequence: Synthetic polypeptide
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGKGTLVTVSS

SEQ ID NO: 304 - protein, 107 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIK

SEQ ID NO: 305 - protein, 445 aa, Artificial Sequence
  Description of Artificial Sequence: Synthetic polypeptide
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGKGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 306 - protein, 213 aa, Artificial Sequence; identical to SEQ ID NO: 300
  Description of Artificial Sequence: Synthetic polypeptide

SEQ ID NO: 307 - protein, 119 aa, Artificial Sequence
  Description of Artificial Sequence: Synthetic polypeptide
QVQLVQSGAEVKKPGSSVKVSCKASGGTFSTYAIGWVRQAPGQGLEWMGGIIPAFGTANYAQKFQGRVTITADESTSTAYMELSSLRSEDTAVYYCARDPVRRSPFDIWGQGTMVTVSS

SEQ ID NO: 308 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDSSNRATGIPARFSGSGSGTDFTLTISSLEPEDFAVYYCQQSFLWPRTFGGGTKVEIK

SEQ ID NO: 309 - protein, 443 aa, Artificial Sequence
  Description of Artificial Sequence: Synthetic polypeptide
QVQLVQSGAEVKKPGSSVKVSCKASGGTFSTYAIGWVRQAPGQGLEWMGGIIPAFGTANYAQKFQGRVTITADESTSTAYMELSSLRSEDTAVYYCARDPVRRSPFDIWGQGTMVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 310 - protein, 213 aa, Artificial Sequence
  Description of Artificial Sequence: Synthetic polypeptide
EIVLTQSPATLSLSPGERATLSCRASQSVSSYLAWYQQKPGQAPRLLIYDSSNRATGIPARFSGSGSGTDFTLTISSLEPEDFAVYYCQQSFLWPRTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 124 aa, Artificial Sequence
  Description of Artificial Sequence: Synthetic polypeptide
QVQLVQSGAEVKKPGASVKVSCKASGYTFSSWYMHWVRQAPGQGLEWMGMINPSGGSTKYAQKFQGRVTMTRDTSTSTVYMELSSLRSEDTAVYYCARDAPFYTWDHYYGMDVWGQGTTVTVSS

SEQ ID NO: 312 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLATGVPSRFSGSGSGTDFTFTISSLQPEDIATYYCQQLYHLPITFGGGTKVEIK

SEQ ID NO: 313 - protein, 448 aa, Artificial Sequence
  Description of Artificial Sequence: Synthetic polypeptide
QVQLVQSGAEVKKPGASVKVSCKASGYTFSSWYMHWVRQAPGQGLEWMGMINPSGGSTKYAQKFQGRVTMTRDTSTSTVYMELSSLRSEDTAVYYCARDAPFYTWDHYYGMDVWGQGTTVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 314 - protein, 213 aa, Artificial Sequence
  Description of Artificial Sequence: Synthetic polypeptide
DIQMTQSPSSLSASVGDRVTITCQASQDISNYLNWYQQKPGKAPKLLIYDASNLATGVPSRFSGSGSGTDFTFTISSLQPEDIATYYCQQLYHLPITFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC